(19) United States
(12) Patent Application Publication
(10) Pub. No.: US 2024/0307283 A1
(43) Pub. Date: Sep. 19, 2024

(54) COMPOSITIONS AND METHODS FOR DECREASING PIGMENTATION

(71) Applicant: The General Hospital Corporation, Boston, MA (US)

(72) Inventors: Elisabeth Roider, Zurich (CH); David Erich Fisher, Newton, MA (US); Inbal Rahamin, Brookline, MA (US)

(21) Appl. No.: 18/576,696

(22) PCT Filed: Jul. 5, 2022
(86) PCT No.: PCT/US2022/073434

§ 371 Date: Jan. 4, 2024

Related U.S. Application Data
(60) Provisional application No. 63/218,427, filed on Jul. 5, 2021.

(51) Int. Cl. A61K 8/42; A61K 8/34; A61K 8/368; A61K 8/37; A61K 8/49; A61K 8/55; A61K 8/60; A61K 8/64; A61Q 19/02

(52) U.S. Cl. CPC A61K 8/42; A61K 8/34; A61K 8/347; A61K 8/368; A61K 8/37; A61K 8/494; A61K 8/4946; A61K 8/4953; A61K 8/4973; A61K 8/498; A61K 8/553; A61K 8/602; A61K 8/64; A61Q 19/02

(57) ABSTRACT

Provided herein are methods for reducing pigmentation, comprising administering to the skin hair, and/or eyes of a subject an effective amount of an nicotinamide nucleotide transhydrogenase (NNT) activator and/or Mitofusin 2 (MFN2) activator, also provided are compositions for use in a method of decreasing pigmentation in the skin, hair, and/or eye of a subject, said method comprising administering the composition to the skin, hair, and/or eye of the subject.

### CLAIM OF PRIORITY

This application claims the benefit of U.S. Patent Application Ser. No. 63/218,427, filed on Jul. 5, 2021. The entire contents of the foregoing are hereby incorporated by reference.

### TECHNICAL FIELD

Provided herein are methods for reducing pigmentation in skin, hair, or eyes comprising administration of an effective amount of an NNT activator and/or MFN2 activator.

### BACKGROUND

Pigmentation of human skin, which confers protection against skin cancer, evolved over one million years ago as a consequence of the evolutionary loss of body hair, human migration to high latitude areas (Jablonski and Chaplin, 2017) and the need to balance skin cancer risk against the skin's ability to maintain vitamin D and folic acid. Human skin color results from the absolute and relative amounts of yellow-orange pheomelanin and black-brown eumelanin (Del Bino et al., 2015). Darker pigmented individuals are more protected from harmful, pro-oncogenic UV radiation by the light scattering and antioxidant properties of eumelanin (Jablonski and Chaplin, 2012). In light Caucasian skin, low eumelanin content and/or a high pheomelanin to eumelanin ratio is present, facilitating the penetration of ultraviolet radiation B (UVB), which is utilized for vitamin D biosynthesis. Vitamin D and folic acid are essential requirements for developmental and post-natal health, and are therefore considered key drivers in the evolution of skin color (Jones et al., 2018).

Even though protection from sunlight has been promoted educationally, melanoma incidence has continued to rise, remaining major personal health and socioeconomic problems (Siegel et al., 2019). Pigment is central to the biology of skin, especially because it dictates how light is absorbed and disseminated in the tissue (Pathak et al., 1962). UV radiation can interact photochemically with DNA to form cyclobutane pyrimidine dimers (CPD) and 6,4-photoproducts and can cause the production of reactive oxygen species (ROS) through multiple mechanisms, thereby increasing the risk of developing skin cancer (Premi et al., 2015). Whereas eumelanin has antioxidant activity, ROS-mediated oxidation of DNA bases and lipid peroxidation is substantially enhanced in mice that produce pheomelanin only (Mitra et al., 2012).

### SUMMARY

As shown herein, increasing the activity of the enzyme nicotinamide nucleotide transhydrogenase (NNT) or Mitofusin 2 (MFN2) decreases pigmentation of human skin in human melanocytes and melanoma cells. Inducing an antioxidant state in human pigmented cells (e.g., melanocytes) using NNT and/or MFN2 activators resulted in potent lightening of human skin and cells (e.g., skin, hair, and/or eye). A new and distinct class of skin, hair, and eye lightening agents (e.g., NNT and/or MFN2 activators) is described herein.

As shown herein, overexpression of NNT and/or MFN2 leads to a reduction in pigmentation. Here we have identified an unexpected and previously unknown role for Mitofusin 2 (MFN2) in the regulation of pigmentation. Overexpression of MFN2 in human melanoma cell lines led to a significant decrease in intracellular melanin and hypopigmentation, followed by a reduction in the expression of the master regulator of melanin synthesis, MITF and its target genes. Tyrp1 and tyrosinase which are key enzymes in the melanin sythesis pathway. The role of MFN2 in pigmentation was further supported by human primary melanocyte data, where overexpression of MFN2 significantly inhibited melanosome maturation. These findings support the use of MFN2 agonists for the treatment of hyperpigmentation disorders, which include both medical and cosmetic applications. MFN2 is a GTPase, which is found in the mitochondrial outer membrane that promotes mitochondrial fusion and subcellular trafficking. Mutations of MFN2 have been reported to mediate the Charcot-Marie-Tooth disease type 2A (CMT2A) syndrome (a form of peripheral neuropathy), and clinical evidence revealed that low MFN2 expression is associated with poor prognosis in many types of cancers. Due to the important role of MFN2 in CMT2A and cancer, several agonists have been published. MFN2 agonists have not been previously shown or suggested to decrease pigmentation or used for any of hyperpigmentation disorders. Consequently, in view of the foregoing, MFN2 agonists can be used as skin, hair, and eye lightening agents.

Thus, provided herein are methods of decreasing pigmentation in the skin, hair, and/or eye of a subject, said method comprising administering to the skin, hair, and/or eye of a subject an effective amount of a composition comprising an effective amount of an nicotinamide nucleotide transhydrogenase (NNT) activator and/or a Mitofusin 2 (MFN2) activator. Also provided are compositions comprising a nicotinamide nucleotide transhydrogenase (NNT) activator and/or a Mitofusin 2 (MFN2) activator for use in a method of decreasing pigmentation in the skin, hair, and/or eye of a subject, said method comprising administering the composition to the skin, hair, and/or eye of the subject.

In some embodiments, the subject has a pigmentation disorder, and/or wishes to decrease the pigmentation in their skin, hair, and/or eye for cosmetic reasons.

In some embodiments, the pigmentation disorder is a localized skin disorder, optionally benign pigmented skin lesions, such as melanocytic nevi, seborrheic keratosis, lentigines, cafe au lait macules, ephelides, congenital dermal melanocytosis; skin cancers, such as melanoma and pigmented basal cell carcinoma; post-inflammatory pigmentation due to prior injury, current or prior inflammatory skin disease such as eczema, especially in dark-skinned individuals, or fixed drug eruption; current or previous superficial skin infection, particularly pityriasis versicolor and erythrasma; chronic pigmentary disorders, particularly melasma and acquired dermal macular hyperpigmentation; phytophotodermatitis or photocontact dermatitis; thickened skin; or a generalized skin disorder, optionally incontinetia pigmenti, Dowling-Degos-syndrome, metabolic and secondary hyperpigmentation; hyperpigmentation in subjects with Addison's disease, haemochromatosis; metastatic melanoma: diffuse melanosis cutis; and in subjects treated with afamelanotide.

A method of decreasing or reducing risk of UVB and/or UVA-induced pigmentation in the skin of a subject in need thereof, said method comprising administering to the skin of a subject in need thereof an effective amount of a composition comprising an effective amount of an NNT activator and/or MFN2 activator, to the skin of a subject prior to, during, and/or after UVB and/or UVA exposure. In some embodiments, the pigmentation disorder is not carotenoderma and/or is not skin cancer.

In some embodiments, the composition comprises an NNT activator, preferably usnic acid, elaidylphosphocholine, diplosalsalate, hexylresorcinol, hexetidine, candesartan, Nigericin, Naproxol, or Ginkgolic acid.

In some embodiments, the composition comprises a MFN2 activator, preferably CpdA and CpdB and derivatives thereof; including Chimera B-A/long (B-A/1); 6-Phenylhexanamide derivatives including derivatives of trans-4-hydroxycyclohexyl)-8-phenylhexanamide such as N-(4-hydroxycyclohexyl)-6-phenylhexanamide (MiM111); Leflunomide; echinacoside (ECH); or minipeptide 1 (MP1).

In some embodiments, the composition is a sunscreen, milk, mask, serum, ointment, paste, cream, lotion, gel, powder, solution, spray, or patch.

In some embodiments, the composition comprises dimethyl sulfoxide (DMSO).

In one aspect, provided herein are methods of decreasing pigmentation in the skin, hair, and eyes of a subject, said methods comprising providing a composition comprising at least one MFN2 agonist to the skin, hair, and/or eye of a subject in an amount sufficient to decrease pigmentation. Such MFN2 agonists include: Leflunomide, see Miret-Casals, Identification of New Activators of Mitochondrial Fusion Reveals a Link between Mitochondrial Morphology and Pyrimidine Metabolism, Cell Chem Biol. 2018 Mar. 15; 25(3):268-278.e4; Cpd A and Cpd B, see Rocha et al, MFN2 agonists reverse mitochondrial defects in preclinical models of Charcot-Marie-Tooth disease Type A, Science 360: 336-41 (2018); The mitofusin activator MiM111, see Franco et al, Burst mitofusin activation reverses neuromuscular dysfunction in murine CMT2A, eLife 9 (2020) e61119 and United States Patent Publication 2020/0345669; Naproxol, (−)-(S)-6-Methoxy-β-Methyl-2-naphthaleneethanol, which is a non-steroidal anti-inflammatory drug, a non-narcotic analgesic and an antipyretic; Candesartan, an angiotensin receptor blocker used mainly for the treatment of high blood pressure and congestive heart failure; Hextidine, which is currently used as an anti-bacterial and anti-fungal agent; and Elaidylphosphocholine, which is a known antineoplastic agent, see Zeng et al, Small molecule induces mitochondrial fusion for neuroprotection via targeting CK2 without affecting its conventional kinase activity, Signal Transduct Target Ther. 2021 Feb. 19; 6(1):71.

In some embodiments, the composition comprises at least one of Leflunomide, Cpd A, CpdB, MiM111, Candesartan, Naproxol, Elaidylphosphocholine, and Hexetidine.

In some embodiments, the subject has a pigmentation disorder, wherein pigmentation in the subject is increased compared to a reference.

In some embodiments, the disorder is characterized by increased pigmentation caused by post inflammatory hyperpigmentation, lentigines, cafe au lait macules, ephelides, seborrhoic keratosis, nevi, melasma, incontinentia pigmenti, dowling-degos-syndrome, and metabolic and secondary hyperpigmentation.

In another aspect, provided herein are methods of decreasing UVB and/or UVA-induced pigmentation in the skin of a subject, said methods comprising providing a composition comprising at least one of Leflunomide, Cpd A, Cpd B, MiM111, Naproxol, Candesartan, Hextidine and Elaidylphosphocholine, or combinations thereof, to the skin of a subject following UVB and/or UVA exposure.

In yet another aspect, provided herein are methods of decreasing pigmentation in the hair of a subject, said methods comprising providing a composition comprising at least one of Leflunomide, Cpd A, Cpd B. MiM111, Naproxol, Candesartan, Hextidine and Elaidylphosphocholine, or combinations thereof, to the hair of a subject in an amount sufficient to decrease pigmentation.

In yet another aspect, provided herein are methods of decreasing pigmentation in the eye of a subject, said methods comprising providing a composition comprising at least one of Leflunomide, Cpd A, Cpd B, MiM111, Naproxol, Candesartan, Hextidine and Elaidylphosphocholine, or combinations thereof, to the eye of a subject in an amount sufficient to decrease pigmentation.

In yet another aspect, provided herein are methods of visible light-induced pigmentation in the skin of a subject, said methods comprising providing a composition comprising at least one of Leflunomide, Cpd A, Cpd B, iM111, Naproxol, Candesartan, Hextidine and Elaidylphosphocholine, or combinations thereof, to the skin of a subject following visible light exposure.

### Definitions

Unless otherwise defined, all technical and scientific terms used herein have the same meaning as commonly understood by one of ordinary skill in the art to which this invention belongs. In case of conflict, the present application, including definitions will control.

A “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “an effective amount” is meant the amount of a required agent or composition comprising the agent to ameliorate the symptoms of increased pigmentation relative to an untreated reference. The effective amount of composition(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is included in the term “effective amount.”

As used herein “a decrease in pigmentation” refers to an amount of pigmentation that is at least about 0.05 fold less (for example 0.1, 0.2, 0.3, 0.4, 0.5, 1, 5, 10, 25, 50, 100, 1000, 10,000-fold or more less) than a reference. “Decreased” as it refers to pigmentation also means at least about 5% less (for example 5, 6, 7, 8, 9, 10, 15, 20, 25, 30, 35, 40, 45, 50, 55, 60, 65, 70, 75, 80, 85, 90, 95, 99 or 100%) than the amount of pigmentation in a reference. As used herein, a reference refers to someone of the same ethnicity, gender and skin type (e.g., skin types 1-8) having normal pigmentation for that ethnicity, gender and skin type. See Fitzpatrick TB: Soleil et peau [Sun and skin]. Journal de Médicine Esthétique 1975; 2:33-34 for a report on skin types 1-6. Amounts can be measured according to methods known in the art for quantifying skin pigmentation. Commonly used methods are absorbance measurements (e.g. OD 490 nm) in cells or upon melanin extraction, visual measurements obtained by a digital camera or a skin-colorimeter, histology using Fontana Masson staining, or mass spectroscopy measurements. Cells or tissue is typically normalized beforehand (e.g., according to equal area, gram of skin or amount of cells).

Unless specifically stated or clear from context, as used herein, the term “about” is understood as within a range of normal tolerance in the art, for example within 2 standard deviations of the mean. “About” is understood as within plus or minus 10% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In this disclosure, “comprises.” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. Nicotinamide Nucleotide Transhydrogenase (NNT) regulates in vitro pigmentation via a redox-dependent mechanism. (A) siNNT increases pigmentation. Quantification of intracellular melanin content of UACC257 cells treated with siControl, siNNT, or siTyrosinase for 72 hours (Left Panel) and human primary melanocytes treated with siControl or siNNT for 96 hours (Right Panel); n=3, analyzed by ordinary one-way ANOVA with Dunnett's post-test (Left Panel) and unpaired, two-sided t-test (Right Panel). Below the graphs, representative cell pellets of the indicated treatment (1×10⁶ cells). (B) Top Schema: Pathways of pheomelanin and eumelanin biosynthesis. DHICA, 5,6-dihydroxyindole-2-carboxylic acid; DHI, 5,6-dihydroxyindole. Graphs: UACC257 melanoma cells were treated with siControl, or siNNT for 5 days and eumelanin and pheomelanin were measured using HPLC techniques (n=3). Absolute pigment levels (Left graph) were analyzed by ordinary two-way ANOVA. The eumelanin/pheomelanin ratio (Right graph) was analyzed by unpaired Student t test. (C) siNNT-induced increased pigmentation of human UACC257 melanoma cells is blocked by NAC (5 mM) or MitoTEMPO (20 μM) (daily treatment for 72 h); n=3, analyzed by ordinary two-way ANOVA with Šidák's post-test. (D-E) Quantification of intracellular melanin content of UACC257 cells treated for 72 hours with siControl, siNNT, silDH1, or silDH1+siNNT (D), or with siControl, siNNT, siPGC1 a, or siNNT+siPGC1 a (E); n=3, analyzed by ordinary one-way ANOVA with Dunnett's post-test. Below the graphs, representative cell pellets (1×10⁶ cells) of the indicated treatments. (F) Overexpression of NNT reduced pigmentation. Melanin content in UACC257 cells that overexpressed NNT (NNT OE) or the corresponding control (Empty Vector) for 12 days; n=3, analyzed by unpaired, two-sided t-test. All data are expressed as mean±SEM; *p<0.05, **p<0.01, ***p<0.0001.

FIGS. 2A-G. Inhibition of NNT enhances melanosome maturation and tyrosinase protein stability via a redox-dependent mechanism. (A) Immunoblot analysis of whole cell lysates from UACC257 melanoma cells 72 hours post-treatment with either siControl or siNNT, showing increased tyrosinase, DCT/TRP2, and TYRP1, but not PMEL17 protein levels. Band intensities were quantified by ImageJ, normalized to P-actin, plotted relative to siControl (n=3), and analyzed by multiple t-tests with the Holm-Siddk post-test. (B-D) siNNT-mediated increased protein stability is blocked by antioxidants. UACC257 cells transfected with siControl or siNNT were treated 24 hours post-transfection with 5 mM NAC (B), 0.1 mM NADPH (C), 20 μM MitoTEMPO (D), or control vehicle for 48 h, followed by CHX treatment. Cells were harvested 0, 1, 2 and 4 h post-CHX treatment for immunoblotting. Band intensities were quantified by ImageJ, normalized to β-actin, and plotted relative to t=0; n=3, analyzed by repeated measures two-way ANOVA with Šidák's post-test (Asterisks indicate significance of siControl/vehicle vs. each of the other three groups). (E) Proteasome inhibitor MG132 inhibits tyrosinase protein degradation upon CHX treatment of NNT-overexpressing UACC257 cells. The cells were treated with DMSO or MG132 (10 μM) for 6 h, followed by CHX treatment for 0, 1, 2 and 4 h and immunoblotting. Band intensities were quantified by ImageJ, normalized to P-actin and plotted relative to t=0; n=3, analyzed by repeated measures two-way ANOVA with Šidák's post-test. (F) Enhanced melanosome maturation induced by siNNT in primary human melanocyte cells is blocked by NAC (5 mM) or MitoTEMPO (20 μM) (daily treatment for 96 h). The ratios of late stages (III+IV) to early stages (I+II) are presented. n=4-5. analyzed by ordinary two-way ANOVA with S\check{i}dák's post-test. (G) Inhibition of melanosome maturation induced by NNT overexpression in primary human melanocytes for 7 days. The ratios of late- to early-stage melanosomes were compared by unpaired, two-sided t-test, n=4 (NNT OE) and n=8 (Empty plasmid). All data are expressed as mean t SEM. *p<0.05, **p<0.01, **p<0.001, ****p<0.0001

FIGS. 3A-H. NNT inhibitors are non-toxic and induce pigmentation of primary melanocytes in vitro and in human skin explants. (A) Murine melanocytes (Melan-A) showed increased melanin content after incubation with 2 mM 2,3BD or DCC, but not after incubation with palmitoyl-CoA; n=3, analyzed by ordinary one-way ANOVA with Dunnett's post-test. (B-C) Treatment of primary human melanocytes with different doses of DCC (B, n=4) or 2.3BD (C, n=6) for 24 hours yielded decreased GSH/GSSG ratios; analyzed by ordinary one-way ANOVA with Tukey's (B) or Dunnett's (C) post-test. (D) A single, one-time topical treatment with 2,3BD (1M or 11M) induces human skin pigmentation after 5 days. Left Panel: Representative images of at least three individual experiments are displayed. Right panel: Reflective colorimetry measurements of skin treated with 2,3BD (higher L* values represent lighter skin tones); n=3, analyzed by ordinary one-way ANOVA with Dunnett's post-test. (E) Fontana-Masson staining of melanin in human skin after 2,3BD (50 mM) (i) and hematoxylin & eosin staining (ii) compared with vehicle control (DMSO). (iii) Supranuclear capping in human keratinocytes of 2,3BD- and vehicle control-treated skin displayed by Fontana-Masson staining. (F) NNT inhibitors, 2,3BD or DCC, applied daily at a 50 mM dose resulted in skin darkening after 5 days. Left Panel: Representative images of three individual experiments are displayed. Right panel: Reflective colorimetry measurements of human skin treated with 2,3BD, DCC, or DMSO vehicle (higher L* values represent lighter skin tones;) n=3, analyzed by ordinary one-way ANOVA with Dunnett's post-test. (G) Immunofluorescence staining for CPD formation in human skin treated with 50 mM 2,3BD for 5 consecutive days. On the last day, skin was irradiated with 1000 mJ/cm² UVB. The results show a protective role for 2,3BD from UVB-induced CPD damage. Representative images of three individual experiments are displayed. Scale bar 50 μM. Quantified results were normalized to the total number of cells; n=3, analyzed by ordinary two-way ANOVA with Šidák's post-test. (H) Measurement of γ-H2AX in human skin revealed no significant toxicity of 2,3BD, while 2,3-BD-induced pigmentation protected from UVB-induced γ-H2AX formation. Representative images of three individual experiments are displayed Scale bar 50 μM. Quantified results were normalized to the total number of cells; n=3, analyzed by ordinary two-way ANOVA with Šidák's post-test. All data are expressed as mean t SEM. *p<0.05, **p<0.01, **p<0.001, ***p<0.0001

FIGS. 4A-E. NNT regulates pigmentation in mice, zebrafish and human pigmentation disorders. (A) Left panel: C57BL/6J mice carrying a 5-exon deletion in the Nnt gene resulting in homozygous loss of NNT activity display increased fur pigmentation compared with C57BL/6NJ wild-type Nnt animals. Right graphs: Mouse fur samples were analyzed for pheomelanin and eumelanin levels by HPLC. n=3, analyzed by multiple t-tests with the Holm-Šid\acute{b}k post-test. (B) Left panel: Zebrafish overexpressing NNT (NNT OE) display decreased pigmentation in individual melanocytes after 5 days. A representative image has been displayed. Results of mean melanocytic brightness, quantified by pixel-based analysis are shown in the graph at right; Empty plasmid (n=11 fish; 72 melanocytes), NNT OE (n=12 fish; 78 melanocytes), analyzed by unpaired, two-sided t-test. (C) Zebrafish with the nnt gene edited using CRISPR/Cas9 (NNT KO) display increased pigmentation after 4 days. A representative image has been displayed. Results of mean melanocytic brightness, quantified by pixel-based analysis are shown in the graph at right; Control (n=42 fish; 120 melanocytes), NNT KO (n=50 fish; 96 melanocytes). (D) Zebrafish treated for 24 hours with either 100 μM 2,3BD or 50 μM DCC display increased darkening after 4 days. A representative image has been displayed. Results of mean melanocytic brightness, quantified by pixel-based analysis are shown in the graph at right; DMSO (n=21 fish; 97 melanocytes), 2,3BD (n=20 fish; 59 melanocytes), DCC (n=18 fish; 57 melanocytes), analyzed by ordinary one-way ANOVA with Dunnett's post-test. (E) Left panel: Human skin specimens from Asian individuals with lentigo or post inflammatory hyperpigmentation were compared to normal skin after staining for NNT, DAPI and Fontana Masson. Representative images of at least 3 samples are displayed (epidermis, E; dermis, D) Graph shows NNT signal intensities normalized to absolute cell numbers (DAPI); n=3, analyzed by ordinary one-way ANOVA with Dunnett's post-test. All data are expressed as mean±SEM; *p<0.05, **p<0.01, **p<0.001, **p<0.0001.

FIGS. 5A-B. Association results for SNPs in the NNT gene with skin color in multiple cohorts. (A) P-values of SNPs from a meta-analysis of skin color (red) combining association results from 4 worldwide cohorts across 462,885 individuals. For each of the 332 SNPs, its location in the NNT gene is shown in the X axis and the negative logarithm of the P-value is shown in the Y-axis. The SNP with the strongest association, rs574878126, is labeled. The adjusted significance threshold is shown with a dashed line. The NNT gene track and a track of regulatory regions obtained from the Ensembl genome browser are shown below. (B) P-values of SNPs from the UK Biobank for sun protection use and ease of skin tanning. For each SNP, its genomic location is shown in the X-axis and negative logarithm of the P-value is shown in the Y-axis. The SNPs with the strongest association for each trait, rs574878126 for sun protection use and rs62367652 for skin tanning, are labeled.

FIGS. 6A-C. Association results and properties of SNPs from various human genetic association analyses. (A-B): Allele frequencies for SNPs in the NNT gene showing most significant associations. (A) Alternative allele frequencies of rs561686035 in various worldwide continental populations, obtained from 1000 Genomes Phase 3. This SNP showed the strongest association in the meta-analysis of skin color and for sun protection use. (B) Alternative allele frequencies of rs62367652 in various worldwide continental populations, obtained from 1000 Genomes Phase 3, are shown. This SNP showed the strongest association for ease of skin tanning (sunburn). (C) Association results for SNPs in the NNT gene with or without conditioning on known pigmentation loci. P-values of SNPs from the Rotterdam Study are shown in this scatterplot. The X-axis represent P-values of SNPs from the standard GWAS analysis of skin pigmentation (not conditioned on any other SNP). P-values from two conditional analyses are plotted on the Y-axis: in darker grey, P-values conditioning on the three known MCIR SNPs; in lighter grey, P-value conditioning on a larger set of known pigmentation SNPs. A diagonal line in black is shown for reference.

FIGS. 7A-L. Inhibition of NNT Increases pigmentation via redox dependent mechanism. (A) siNNT-induced increased pigmentation in human SK-MEL-30 melanoma cells is dependent on tyrosinase and reactive oxygen species. Left panel: Representative lysates from SK-MEL-30 cells following treatment with siControl, siNNT, siNNT+siTyrosinase (siTYR), or siNNT+5 mM NAC. Right panel: Quantification of intracellular melanin content in SK-MEL-30 cells; n=3. analyzed by ordinary one-way ANOVA with Dunnett's post-test. (B, C) qRT-PCR analysis of NNT (B) and immunofluorescence of NNT (C) in primary human melanocytes treated with siControl or siNNT for 96 hours. IF staining of human NNT and nuclei (DAPI) are shown. Scale bar 50 μM. Relative NNT mRNA levels and fluorescent intensities (n=3) were analyzed by unpaired, two-sided t-tests. (D) Immunoblot analysis of NNT expression in UACC257 human melanoma cells. Band intensities were quantified by ImageJ, normalized to f-actin and plotted relative to siControl; n=3, analyzed by unpaired, two-sided t-test. (E) Treatment of UACC257 cells with siNNT for 24 hours resulted in increased NADPH/NADP (Left panel, n=9) and decreased GSH/GSSG (Right panel, n=6) ratios. The data were analyzed by multiple t tests with the Holm-Šidák post-test. (F) UACC257 melanoma cells were treated with siControl or siTyrosinase for 5 days and eumelanin and pheomelanin were measured using HPLC techniques (n=3). Absolute pigment levels (Left graph) were analyzed by ordinary two-way ANOVA, separately for eumelanin and pheomelanin. The eumelanin/pheomelanin ratio (Right graph) was analyzed by unpaired Student t test (G). Increased ROS in UACC257 cells following 48 hours of siNNT or silDH1 treatment, but not after 48 hours of siPGCa treatment. IF images of ROS indicator DCFDA and nuclei (DAPI), representative of five experiments, are displayed. Quantified results were normalized to the total number of cells and analyzed by ordinary one-way ANOVA with Šidák's post-test. (H) Increase of melanin content by siNNT is blocked by cotreatment with NADPH. Intracellular melanin content was quantified in UACC257 cells treated with siControl or siNNT for 72 hours, with 0.1 M NADPH or Vehicle (Tris-HCl, pH 8.0) added after the first 24 hours. n=3, analyzed by ordinary two-way ANOVA with Šidák's post-test. (1) Immunoblot analysis of IDH1 in UACC257 cells treated with siControl, siNNT, silDH1, or siNNT+silDH1 together for 72 hours. Band intensities were quantified by ImageJ, normalized to f-actin (n=3), and analyzed by ordinary one-way ANOVA with Dunnett's post-test. (J) qRT-PCR analysis of NNT, IDH1, and PGC1α mRNAs in UACC257 cells treated with siRNA for one of those genes or siControl. qRT-PCR data are normalized to RPL11 RNA and RNA levels are presented as fold change relative to siControl; (n=3), analyzed by ordinary one-way ANOVA with Dunnett's post-test, followed by the Bonferroni correction for three ANOVA analyses. (K, L) Overexpression of NNT in the UACC257 cell line: (K) qRT-PCR analysis of NNT mRNA five days post transfection; n=3, analyzed by unpaired, two-sided t-test. (L) Overexpression of NNT resulted in decreased NADPH/NADP (Left panel, n=8) and increased GSH/GSSG (Right panel, n=4-6) ratios, analyzed by multiple t-tests with the Holm-Šidák post-test. All data are expressed as mean t SEM; *p<0.05, **p<0.01, ***p<0.001, ****p<0.0001.

FIGS. 8A-N. NNT does not impact TYR mRNA expression levels and acts independently of the cAMP pathway. (A) Tyrosinase activity increase following siNNT in UACC257 melanoma cells; n=4. analyzed by unpaired, two-sided t-test (B) Diagram of the “Tanning Pathway”. Briefly, UV exposure results in DNA damage and activation of P53 in keratinocytes. POMC is transcriptionally activated by P53 and the pro-protein is cleaved to α-MSH, which is secreted from the keratinocyte. α-MSH binds to MC1R in the melanocyte membrane, resulting in an increase in cAMP and activation of PKA. Active PKA results in an increase of MITF, activated transcriptionally by CREB. MITF transcriptionally regulates pigmentation enzymes such us TYRP1, TRP2 and tyrosinase. (C) Immunoblot analysis of MITF in UACC257 cells transfected with siNNT or siControl for 72 h. Band intensities were quantified by ImageJ, normalized to μ-actin, plotted relative to siControl values (n=3), and analyzed by unpaired, two-sided t-test. (D-F) Analyses of UACC257 cells stably expressing secreted luciferase under the TRPM1 promoter and SEAP under the CMV promoter. The cells were treated with either siControl, siNNT, or siMITF (n=3): (D) qRT-PCR analysis of NNT, mMITF and TYRP1 72 hours post siRNA transfection. Data were normalized to RPL11 RNA, analyzed by unpaired, two-sided t-test (NNT) or ordinary one-way ANOVA with Dunnett's post-test (mMITF and TYRP1). (E) Luciferase secretion normalized to secreted SEAP 72 hours post siRNA transfection, showing decreased luciferase activity following siMITF and siNNT, analyzed by ordinary one-way ANOVA with Dunnett's post-test; representative cell pellets (1×10⁸ cells) are below the graph. (F) Luciferase secretion normalized to secreted SEAP 24, 48 and 72 hours post siRNA transfection was analyzed by repeated measures two-way ANOVA with Midk's post-test. (G) qRT-PCR analysis of NNT, MITF, TYRP1, TRP2/DCT, NNT, tyrosinase, and POMC in UACC257 cells 72 hours post transfection of siNNT or siControl. Data were normalized to RPL11 RNA, presented as fold change relative to siControl (n=3), and analyzed by multiple t-tests with the Holm-Šidák post-test. (H) cAMP content of UACC257 cells transfected with siNNT or siControl for 48 h, measured by cAMP ELISA and normalized to siControl cells; n=3, analyzed by unpaired, two-sided t-test. (1) Primary human melanocytes were starved for 24 hours and Forskolin (FSK; 20 μM) was added to the medium for 2 hours. qRT-PCR analysis of NNT was performed with MITF as a positive control for the treatment. The data were normalized to RPL11 RNA (n=3) and analyzed by multiple t-tests with the Holm-Šidák post-test. (J) No change in NNT mRNA upon UVB. Abdominal skin was irradiated with 1 J/cm² UVB, skin was collected at 0, 24, 48 and 72 hours post UVB, and qRT-PCR analysis of NNT was performed. The data were normalized to RPL11 RNA and presented as fold change relative to t=0. n=5-8 (two different donors), analyzed by ordinary one-way ANOVA with Dunnett's post-test. (K) Immunoblots of P53 and μ-actin in UACC257 cells following siControl or siNNT treatment for 72 hours. (L) Immunoblot of NNT and β-actin in UACC257 melanoma cells (Left panel), daily treatment with NAC (5 mM), MitoTEMPO (20 μM) and H₂O₂(100 μM) for 72 hours (n=3) analyzed by ordinary one-way ANOVA with Tukey post-test. (M) Immunoblots of tyrosinase and f-actin in UACC257 melanoma cells (Left panel), showing decreased tyrosinase protein levels following overexpression of NNT for 12 days. Band intensities were quantified by ImageJ, normalized to β-actin and plotted relative to siControl values (Right Panel). (n=3), analyzed by unpaired, two-sided t-test. (N) qRT-PCR analysis of MITF, TYRP1 and tyrosinase mRNAs in UACC257 cells that overexpressed NNT (NNT OE), compared to control (Empty Vector). The data were normalized to RPL11 RNA (n=3) and analyzed by ordinary one-way ANOVA with Dunnett's post-test, followed by the Bonferroni correction for three ANOVA analyses.

FIGS. 9A-J. NNT knockdown enhances melanosome maturation, melanosome-mitochondria proximity and pigmentation by NNT knockdown. (A) Enhanced melanosome maturation induced by siNNT in human primary melanocyte cells is blocked by NAC (5 mM) or MitoTEMPO (20 μM) (daily treatment for 96 h). The number of melanosomes per um² in the classified stages is represented. n=4-5 cells, analyzed by ordinary two-way ANOVA with Šidák's post-test (B) The total number of melanosomes per um² in primary human melanocytes is not altered by siNNT and/or daily treatment with NAC (5 mM) or MitoTEMPO (20 μM) for 96 hours (Left graph, n=8-10, analyzed by ordinary one-way ANOVA with Dunnett's post-test) or by overexpression of NNT (Middle graph, n=8-10), analyzed by unpaired, two-sided t-test. The total numbers of mitochondria per um² by overexpression of NNT (Right graph, n=5) is not altered. (C) Measurements of proximities between melanosomes and mitochondria were quantified in FIJI (ImageJ) by applying a customized macro to TEM micrographs (n=100 events per condition). Melanosome-mitochondria proximities closer than 20 nm are considered melanosome-mitochondria close appositions/contacts. Right panel: FIJI graphical user interface showing a TEM micrograph of mitochondria (m) and a melanosome (*) with a yellow line indicating the Euclidean distance between melanosome and mitochondrion surfaces, quantified with a customized macro to measure distances between two surfaces. Scale bar 400 nm. Table shows the percentages and, in parentheses, the fractions of melanosome-mitochondria proximities that were <20 nm. Denominators are the total number of measurements (events) performed in each group. Adjusted P values were determined by pairwise F-tests of the control group to each of the other groups, followed by the Bonferroni correction for three comparisons. (D-E) The total numbers of melanosomes (D) and mitochondria (E) per um² in primary human melanocytes is not altered; n=5 cells, analyzed by ordinary two-way ANOVA with Šidák's post-test. (F) MFN2 enables siNNT-mediated pigmentation. Top panel: Quantification by spectrophotometry of intracellular melanin content of UACC257 human melanoma cells treated with siControl, siNNT, siMFN2+siNNT, or siMFN2 for 72 hours. n=3, analyzed by ordinary one-way ANOVA with Dunnett's post-test. Bottom panel: Representative cell pellets (10⁵ cells). (G) Immunoblot analysis of MFN2 expression in UACC257 human melanoma cell lines. Band intensities (n=3) were quantified by ImageJ, normalized to β-actin, and analyzed by ordinary one-way ANOVA with Dunnett's post-test. (H) qRT-PCR analysis of MFN2 in primary human melanocytes that were transfected with siMFN2. The data were normalized to RPL11 RNA, plotted relative to the control (n=3), and analyzed by unpaired, two-sided t-tests (1) siMFN2 resulted in accumulation of large autophagosomes (white arrows), containing numerous melanosomes (arrowheads), in normal human melanocytes. Scale bar 2 μm. (J) Immunoblot analysis of LC3B in UACC257 cells treated with siMFN2, siNNT, siMFN2+siNNT, or siControl for 72 hours. Band intensities were quantified by ImageJ and normalized to μ-actin. The ratios of LC3BII to LC3BI were plotted (n=3) and analyzed by ordinary one-way ANOVA with Dunnett's post-test. All data are expressed as mean±SEM; *p<0.05, **p<0.01, ***p<0.001, ****p<0.0001.

FIGS. 10A-F. NNT inhibitors are non-toxic in vitro. (A) Chemical formulas of all three published NNT inhibitors. (B) Viability measurements showed no significant toxicity after treatment of human melanocytes, dermal fibroblasts, and keratinocytes with up to 10 μM of DCC, Palmitoyl-CoA, or 2,3BD. (C) Treatments with different doses of DCC (Left graph) or 2,3 BD (Right graph) had no impacts on cell viability. The data were plotted relative to vehicle treatment (0) and analyzed by ordinary one-way ANOVA with Dunnett's post-test (n=4). (D) Intracellular melanin content normalized to total protein levels in primary human melanocytes that were treated with siControl or siNNT for 24 hours, followed by incubation with 2.3 BD (2 mM) or DMSO vehicle for 72 hours. n=3, analyzed by ordinary one-way ANOVA with Šidák's post-test. (E) Treatments with different doses of DCC had no impact on cell viability (Right graph), but resulted in decreased GSH/GSSG ratios (Left graph) in the UACC257 human melanoma cell line. n=4. analyzed by ordinary one-way ANOVA with Tukey's (Left graph) or Šidák's (Right graph) post-test. (F) Fontana-Masson staining of melanin in human abdominal skin 5 days after a single treatment of 2,3BD (1M), showing supranuclear capping (Black arrows) in keratinocytes of 2,3BD-treated skin. Scale bar 50 μM. All data are expressed as mean±SEM; *p<0.05, *p<0.01, ****p<0.0001.

FIGS. 11A-D. NNT regulates pigmentation in mice, zebrafish and human pigmentation disorders. (A) Agarose gel showing PCR genotyping of DNA from C57BL/6J mice (single 743 bp product indicates homozygous 5-exon deletion in the Nnt gene) and C57BL6NJ mice (single 570 bp product indicates homozygous wild type Nnt gene). (B) Modification of NNT sites in zebrafish using WT SpCas9. Editing was assessed by next-generation targeted amplicon sequencing. (C) Zebrafish overexpressing NNT (NNT OE) or empty plasmid were treated at 3 days post fertilization with 100 μM of 2,3BD or vehicle for 24 hours. A representative image has been displayed. Results of mean melanocytic brightness, quantified by pixel-based analysis are shown in the graph at right; Empty plasmid (n=12 fish; 30 melanocytes), NNT OE (n=10 fish; 24 melanocytes), Empty plasmid+2,3 BD (n=8 fish; 30 melanocytes), NNT OE+2,3BD (n=11 fish; 31 melanocytes), analyzed by ordinary one-way ANOVA with Dunnett's post-test (D) Representative images of the specific areas of hyperpigmentation in human lentigo-affected skin after staining for NNT (left image, red) or Fontana Masson (right image). Graph at right shows NNT signal intensities in melanocytes of healthy and lesional skin. n=9 (bars indicate means), analyzed by ordinary one-way ANOVA with Dunnett's post-test.

FIGS. 12A-B. In vitro depigmenting effects of NNT activators demonstrated in pigementary cells. The depigmenting effects of Acetylsalicylic Acid (ASS). Usnic acid, 4-hexylresorcinol, candesartan, Nigericin, and Ginkgolic acid to depigment were evaluated in (A) mouse B16 melanoma cells and (B) mouse melan-A melanocytes.

FIGS. 13A-B. NNT activators display skin lightening effects in human skin explants. The depigmenting effects of (A) ASS, Usnic acid, 4-hexylresorcinol, candesartan, Nigericin, and Ginkgolic acid, and (B) elaidylphosphocholine, hexitidine, and naproxol, to depigment were evaluated in human skin explants. (C) NNT activators display lightening effects in human skin explants, as shown by Fontana Masson and H&E staining.

FIG. 14. NNT activators can prevent UVB-driven pigmentation of skin. The ability of various concentrations of NNT activators ASS, Usnic Acid, Nigericin, Gingkolic Acid, Candesartan, and 4-Heylresorcinol to prevent UVB driven pigmentation was tested with application of 150 mJ/cm².

FIG. 15. NNT activators display skin lightening effects in human skin. Results of treatment with Hexetidine 100 uM: 15 days, 2× per day, in a skin type 2 individual.

FIGS. 16A-E. Effects of MFN2 modulation on pigmentation. (A) Overexpression of MFN2 reduced pigmentation. UACC257 cells that overexpress MFN2 (MFN2 OE) or the corresponding Empty vector (EP) control for 14 days were then transfected with either siControl or siNNT for 72 hours and intracellular melanin content was quantified and normalized to protein levels. n=3, analyzed by ordinary one-way ANOVA. Below the graph are representative cell pellets (106 cells) from the indicated treatments(B) UACC257 cells stably overexpressing HA-MFN2 were transfected with siControl or siNNT and immunoblotting for tyrosinase, HA tag and β-actin was performed. Band intensities were quantified by ImageJ, normalized to β-actin, plotted relative to siControl (n=3), and analyzed by ordinary one-way ANOVA with Dunnett's post-test (C). Decrease in the ratio of late- to early-stage melanosomes in primary human melanocytes that overexpress MFN2 for 7 days, plotted (n=4-8) and compared by unpaired, two-sided t-test. (D) qRT-PCR analysis of MITF, TYRP1 and tyrosinase mRNAs in UACC257 cells that overexpress MFN2 (MFN2 OE) or NNT (NNT OE), compared to control (Empty Vector). The data were normalized to RPL11 RNA (n=3) and analyzed by ordinary one-way ANOVA with Dunnett's post-test, followed by the Bonferroni correction for three ANOVA analyses. (E) Immunoblot analysis of tyrosinase levels in UACC257 cells treated for 72 hours with siControl, siNNT, siMFN2, or siMFN2+siNNT. Band intensities (n=3) were quantified by ImageJ, normalized to β-actin and plotted relative to siControl values (Right Panel). n=3, analyzed by ordinary one-way ANOVA with Dunnett's post-test.

### DETAILED DESCRIPTION

Melanocytes located in the basal epidermal layer produce melanin within subcellular organelles called melanosomes. Melanosomes mature from an early, unpigmented state (stages I-II) towards a late, pigmented state (stages III-IV). Early-stage melanosomes are recognized by proteinaceous fibrils within the melanosomal lumen. In the late stages melanin is gradually deposited on the fibrils until complete pigmentation is achieved (Raposo and Marks, 2007). These mature melanosomes are ultimately transferred to keratinocytes (Park et al., 2009) where they coalesce in a supranuclear location on the sun-facing side. Current data suggests that UV radiation triggers tanning by causing DNA damage that increases p53 in human keratinocytes, thereby stimulating the synthesis of pro-opiomelanocortin (POMC) and its cleavage products including α-melanocyte-stimulating hormone (α-MSH). Secreted α-MSH binds to the melanocortin 1 receptor (MCIR) on melanocytes, resulting in cAMP-mediated induction of the microphthalmia-associated transcription factor (MITF), which directly stimulates transcription of the genes for tyrosinase-related protein 1 and 2 (TYRP-1 and DCT) (Lo and Fisher, 2014) and tyrosinase, which drive melanosome maturation (Paterson et al., 2015) and increased production of eumelanin (lozumi et al., 1993).

The enzyme nicotinamide nucleotide transhydrogenase (NNT) is located in the inner mitochondrial membrane. It regulates mitochondrial redox levels by coupling hydride transfer between f-nicotinamide adenine dinucleotide NAD(H) and f-nicotinamide adenine dinucleotide 2′-phosphate NADP (+) to proton translocation across the inner mitochondrial membrane (Earle and Fisher, 1980; Rydstrom et al., 1970; Zhang et al., 2017). The mitofusion 2 protein MFN2 is a mitochondrial membrane protein that plays a central role in regulating mitochondrial fusion and cell metabolism. More specifically, MFN2 is a dynamin-like GTPase embedded in the outer mitochondrial membrane, which in turn affects mitochondrial dynamics, distribution, quality control, and function.

Understanding the interplay between melanin and redox metabolism is important, since many cosmetics are supplemented with antioxidants, presumably aiming to provide some form of skin protection. Even though antioxidants including glutathione are used in Asia for human skin lightening (Sonthalia et al., 2016) (Rachmin et al., 2020), potential underlying mechanism(s) of action are incompletely understood. In addition, much pigmentation research has been done in Caucasians, resulting in a significant lack of knowledge of non-Caucasian skin pigmentation. Moreover, the exact mechanisms of many pigmentation disorders, such as postinflammatory hyperpigmentation and lentigines, have not been fully elucidated. As a consequence, currently available treatments are neither specific nor very successful. Identification of an MITF- and UV-independent mechanism of skin pigmentation offers new skin cancer prevention and/or pigmentation disorder treatment strategies.

The present study identified (i) the existence of a distinct redox-dependent. UV- and MITF-independent skin pigmentation mechanism; (ii) a new role for the mitochondrial redox-regulating enzyme NNT in altering pigmentation by regulating tyrosinase protein stability and melanosome maturation via a redox-dependent and MITF-independent mechanism; (iii) a class of topical compounds that activate NNT and/or MFN2 and yield human skin, hair, and eye lightening.

While hundreds of genes have been shown to affect pigmentation in model organisms (e.g., the Color Genes database: espcr.org/micemut/), few have been associated with skin color variation in humans (Martin et al., 2017). Whereas most previous pigmentation research has been performed in individuals of European ancestry, recent genome-wide association studies (GWAS) in non-Europeans (Arjinpathana and Asawanonda, 2012; Crawford et al., 2017; Hysi et al., 2018; Lin et al., 2018; Martin et al., 2017) emphasized the complex nature of human skin pigmentation. Evidence is evolving that, in addition to certain major regulators such as pigmentation factors (e.g., TYR and MITF), many other genes may impact skin pigmentation and an individual's unique skin color. It is thus plausible that factors involved in redox metabolism, such as NNT, may be responsive to environmental changes such as UV exposure or inflammation. Increasing eumelanin levels as a response to ROS-inducing events might have been beneficial during evolution by maintaining the cutaneous redox equilibrium. Even though an interplay between oxidative stress and skin pigmentation was suspected (Arjinpathana and Asawanonda, 2012), neither the exact mechanism nor ways to clinically target this mechanism have yet been established. The results presented here demonstrate the existence of a conserved, redox-dependent pigmentation mechanism affecting eumelanin levels, which can be modified by changing NNT enzyme activity in an MITF-independent manner, offering novel potential clinical applications for significant groups of patients.

Intermediately pigmented human melanoma cells and melanocytes were tested, which exhibited decreased pigmentation after treatment with NNT- or MFN2-activating compounds and decreased pigmentation after overexpression of NNT or MFN2. Pigmentation genes and intermediates involved in the classic UVB-cAMP-MITF-dependent pigmentation pathway were not affected. However, in vitro experiments suggested that increased pigmentation is dependent on cytosolic and mitochondrial ROS as well as tyrosinase, involves increased tyrosinase-related genes and inhibition of proteasome-mediated tyrosinase protein degradation, and is associated with melanosome maturation. The possibility of additional redox/NNT-driven mechanisms of skin pigmentation, melanosome transport, or direct melanin oxidation will be valuable to investigate in future studies.

Murine and zebrafish models were used to investigate the effect of NNT-mediated redox changes in vivo. We first observed darker pigmentation in NNT-defective C57BU6J mice compared with NNT-competent C57BL/6NJ. Moreover, we engineered a zebrafish model producing melanocytes with or without expression of NNT, which uncovered an additional in vivo link between NNT depletion and darker pigmentation.

The experimental data herein demonstrate that the NNT and MFN2 genes are involved in skin pigmentation in fish, rodents and humans. This is further supported by the observed associations between genetic variants in the NNT gene region and variation of normal skin pigmentation among diverse human cohorts. We found significant associations with several markers within the NNT gene in a meta-analysis combining four diverse worldwide cohorts: the European-ancestry Rotterdam cohort, which used a physician-based 6-level skin color grading system (Jacobs et al., 2015); the UK Biobank cohort, which has a similar genetic background as the Rotterdam Study and a self-reported 6-level pigmentation phenotype; a Latin American dataset (CANDELA) based on a quantitative evaluation of skin pigmentation; and a smaller East & South African cohort with quantitative pigmentation measurement. Interestingly, associations were also observed for ease of skin tanning and sun protection use in the UK Biobank dataset. The derived alleles in each case corresponded to a reduced NNT expression in skin tissues and were associated with darker skin color, less sunburn, and less sun protection use, which is consistent with the previously identified role of NNT in redox metabolism and its roles shown here in reactions to UV light and pigment regulation. This is in line with our findings that NNT acts as a gatekeeper in the oxidative stress-mediated skin pigmentation pathway, independent of MITF-driven pathway, contributing to human skin color, tanning and the pathogenesis of different oxidative stress-mediated skin disorders (Huls et al., 2018) such as lentigo and postinflammatory hyperpigmentation.

### Methods of Use

Provided herein are methods for reducing pigmentation in skin, hair, and eyes comprising administration of an effective amount of an NNT activator and/or MFN2 activator. The present methods lighten skin independent of UV exposure, and therefore can act on healthy individuals (e.g., for cosmetic purposes) and on hyperpigmented skin, e.g., affected by a pigmentation disorder (e.g., for a therapeutic). The methods can be used, e.g., for cosmetic purposes in subjects who wish to lighten the color of their skin, hair, or eyes; or for therapeutic purposes, e.g., for treating a number of pigmentation disorders (i.e., disorders associated with hyperpigmentation), which are among the most common reasons for dermatological consultations (Cestari et al., 2014). Although these disorders are usually not life-threatening, they often have an impact on the quality of life of affected individuals (Taylor et al., 2008). Such pigmentation disorders include localized and systemic disorders.

Exemplary localized skin disorders include: benign pigmented skin lesions, such as melanocytic nevi (e.g., nevus of Ota), seborrheic keratosis, lentigines, cafe au lait macules, ephelides, congenital dermal melanocytosis (Mongolian spot); skin cancers, such as melanoma and pigmented basal cell carcinoma; post-inflammatory pigmentation due to prior injury, current or prior inflammatory skin disease such as eczema, especially in dark-skinned individuals, or fixed drug eruption; current or previous superficial skin infection, particularly pityriasis versicolor and erythrasma; chronic pigmentary disorders, particularly melasma and acquired dermal macular hyperpigmentation; phytophotodermatitis or photocontact dermatitis; thickened skin eg, acanthosis nigricans or ichthyosis. Generalized skin disorders include incontinetia pigmenti, Dowling-Degos syndrome, metabolic and secondary hyperpigmentation; hyperpigmentation in subjects with Addison's disease, haemochromatosis; metastatic melanoma: diffuse melanosis cutis; and in subjects treated with afamelanotide. In some embodiments, the pigmentation disorder is not carotenoderma and/or is not skin cancer.

In addition, UV exposure induces skin pigmentation and melanin generation, which can be reduced by pre-exposure treatment, concurrent treatment, or post-exposure treatment with NNT/MFN2 activators (aims towards preventing tanning). Thus the present methods can be used to inhibit the UVA/UVB-driven darkening of skin, particularly in individuals with fair skin (e.g., Fitzpatrick 1-2). Furthermore, NNT activators reduce oxidative stress and therefore decrease skin cancer risk.

In some embodiments, the subject has Fitzpatrick skin type 1. In some embodiments, the subject has Fitzpatrick skin type 2. In some embodiments, the subject has Fitzpatrick skin type 3. In some embodiments, the subject has Fitzpatrick skin type 4. In some embodiments, the subject has Fitzpatrick skin type 5. In some embodiments, the subject has Fitzpatrick skin type 6.

Fitzpatrick skin type

| Skin type | Typical features | Tanning ability |
|---|---|---|
| I | Pale white skin, blue/green | Always burns, does not tan |
| | eyes, blond/red hair | |
| II | Fair skin, blue eyes | Burns easily, tans poorly |
| III | Darker white skin | Tans after initial burn |
| IV | Light brown skin | Burns minimally, tans easily |
| V | Brown skin | Rarely burns, tans darkly easily |
| VI | Dark brown or black skin | Never burns, always tans darkly |

In general, the methods described herein include administering an effective amount of a composition comprising an NNT activator and/or MFN2 activator. An “effective amount” as used herein is an amount sufficient to reduce pigmentation of skin, hair, and eye(s) (where lightening is desired) or to reduce UVB-induced darkening of skin, hair, and eye(s). Exemplary doses include those shown herein.

### NNT Activators

NNT activators include small molecules and other compounds that induce the enzymatic activity of NNT, which promotes formation of NADPH and thereby enhances intracellular protection against oxidative stress, to thereby prevents generation of melanin (specifically eumelanin and pheomelanin). A number of NNT activators are known in the art and suitable for use in the present methods and compositions, including usnic acid, elaidylphosphocholine, diplosalsalate, hexylresorcinol, hexetidine, candesartan, Nigericin, Naproxol, and Ginkgolic acid. see, e.g., Meadows at al., Journal of Biomolecular Screening 16(7):734-43; 2011. In some embodiments, the NNT inhibitor is usnic acid, diplosalsalate, or Ginkgolic acid. In some embodiments, the NNT activator is not hexylresorcinol, 4-n-butylresorcinol, or nigericin. In some embodiments, the methods and compositions comprise hexylresorcinol, 4-n-butylresorcinol, or nigericin and another NNT activator and/or a MFN2 activator.

### MFN2 Activators

MFN2 activators include small molecules and other compounds that alter the mitochondria-melanosomal ultrastructure in a way that disrupts pigment and specifically (eu- and pheomo-) melanin formation. A number of MFN2 activators are known in the art and suitable for use in the present methods and compositions, including small molecules such as CpdA and CpdB and derivatives thereof including Chimera B-A/long (B-A/I) (see, e.g., Rocha et al., Science 360, 336-341 2018); 6-Phenylhexanamide derivatives (see, e.g., Dang et al., J. Med. Chem. 2020, 63, 7033-7051; PCT/US2020/014784) including derivatives of (trans-4-hydroxycyclohexyl)-6-phenylhexanamide such as N-(4-hydroxycyclohexyl)-6-phenylhexanamide (MiM111) (see, e.g., PCT/US2019/046356); Leflunomide (see, e.g., Miret-Casals et al., Cell Chem Biol. 2018 Mar. 15; 25(3):268-278.e4); echinacoside (ECH) (see, e.g., Zeng et al., Small molecule induces mitochondrial fusion for neuroprotection via targeting CK2 without affecting its conventional kinase activity. Signal Transduct Target Ther. 2021 Feb. 19; 6(1):71, and see also CN102670436B); and peptides, e.g., minipeptide 1 (MP1, a minipeptide made up of residues 367-384 of MFN2, optionally comprising a cell penetrating peptide such as TAT, see, e.g., Franco et al., Nature. 2016 Dec. 1; 540(7631):74-79). Leflunomide, C12H9F3N202, is a derivative of isoxazole used for its immune-suppressive and anti-inflammatory properties. As a prodrug, leflunomide is converted to an active metabolite, A77 1726, which blocks dihydroorotate dehydrogenase, a key enzyme of de novo pyrimidine synthesis, thereby preventing the expansion of activated T lymphocytes. It also inhibits various protein tyrosine kinases, such as protein kinase C (PKC), thereby inhibiting cell proliferation. It has been uses as an immunomodulatory agent used in treatment of rheumatoid arthritis and psoriatic arthritis. Candesartan is known in the art as 1-((2′-(1H-Tetrazol-5-yl)-[1,1′-biphenyl]-4-yl)methyl)-2-ethoxy-1H-benzo[d]imidazole-7-carboxylic acid. Candesartan is a synthetic, benzimidazole-derived angiotensin II receptor antagonist prodrug with antihypertensive activity. Naproxol is known in the art as (−)-2-(8-Methoxy-2-naphthyl)-1-propanol. Naproxol is a nonsteroidal anti-inflammatory drug. Elaidylphosphocholine is [(E)-octadec-9-eny] 2-(trimethylazaniumyl)ethyl phosphate. Hexetidine is known in the art as 1,3-bis(2-ethylhexyl)-5-methyl-1,3-diazinan-5-amine or C₂H4₅N3. Hexetidine is a bactericidal and fungicidal antiseptic.

In some embodiments, the MFN2 inhibitor is MiM111. In some embodiments, the methods and compositions do not include echinacoside, or have less than 25%, less than 20%, or less than 10% echinacoside. In some embodiments, the methods and compositions include echinacoside and another MFN2 activator and/or an NNT activator.

### Compositions

The methods described herein include the use of compositions comprising or consisting of an NNT activator and/or MFN2 activator (also referred to herein as “skin lightening agents” or “skin, hair, and/or eye lightening agents”) as an active ingredient; the pharmaceutical compositions are also provided herein as well as methods of use thereof.

Compositions including pharmaceutical compositions are typically formulated to be compatible with its intended route of administration. Preferably, the present methods include topical administration, though intradermal or subcutaneous administration can also be used. Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. The compositions can also comprise cosmetically-acceptable carriers or vehicles and any optional components. A number of such cosmetically acceptable carriers, vehicles and optional components are known in the art and include carriers and vehicles suitable for application to skin, hair, or eyes. In some embodiments, e.g., for administration to skin, the compositions can be in the form of sunscreens, milks, masks, serums ointments, pastes, creams, lotions, gels, powders, solutions, sprays, or patches. In some embodiments, e.g., for administration to hair, the compositions can be in the form of shampoos, conditioners, pastes, balms, masks, sprays, oils, or other liquid or semi-liquid form. In some embodiments, formulations of the compositions can further contain saturated or unsaturated fatty acids such as stearic acid, palmitic acid, oleic acid, palmito-oleic acid, cetyl or oleyl alcohols, stearic acid being particularly preferred. Such compositions can also contain a non-ionic surfactant, for example, polyoxy-40-stearate. In some embodiments, the active component is admixed under sterile conditions with a pharmaceutically acceptable excipient and any needed preservatives or buffers as may be required. In some embodiments, e.g., for administration to the eye, ophthalmic formulations, e.g., ointments or eye drops are also contemplated herein.

Supplementary active and inactive compounds can also be incorporated into the compositions, e.g., absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof. In addition, the composition can comprise one or more oily substances, waxes, emulsifiers, coemulsifiers, solubilizers, cationic polymers, film formers, superfatting agents, refatting agents, foam stabilizers, stabilizers, active biogenic substances, preservatives, preservation boosting ingredients, anti-fungal substance, anti-dandruff agents, dyes or pigments, particulate substances, opacifiers, abrasives, absorbents, anticaking agents, bulking agents, pearlizing agents, direct dyes, perfumes or fragrances, carriers, solvents or diluents, propellants, functional acids, active ingredients, skin-brightening agents, self-tanning agents, exfoliants, enzymes, anti-acne agents, deodorants and anti-perspirants, viscosity modifiers, thickening and gelling agents, pH adjusting agents, buffering agents. anti-oxidants, chelants, astringents, sunscreens, sun protection agents, UV filters, skin conditioning agents, emollients, humectants, occlusive agents, pediculocides, anti-foaming agents, flavouring agents, electrolytes, oxidizing agents and reducing agents.

The skin, hair, and/or eye lightening agents described herein can be administered alone or as a component of a cosmetic or pharmaceutical formulation. In specific embodiments, the amount of skin, hair, and/or eye lightening agent is between about 5 uM to about 50 mM in the composition. Single or multiple administrations of compositions can be given depending on for example: the dosage and frequency as required, the degree and amount of pigmentation, and the like.

The compounds can be formulated for administration, in any convenient way for use in human medicine. In practicing this invention, the compositions can be delivered transdermally, by a topical route, formulated as applicator sticks, solutions, suspensions, emulsions, gels, creams, ointments, pastes, jellies, paints, powders, and aerosols.

Formulations of the compositions can include those suitable for topical administration to the skin, hair, and/or eye. The formulations may conveniently be presented in unit dosage form and may be prepared by any methods well known in the art of pharmacy. The amount of active ingredient (e.g., skin, hair, and/or eye lightening agents described herein) which can be combined with a carrier material to produce a single dosage form will vary depending upon the host being treated, the particular mode of administration, e.g., intradermal. The amount of active ingredient which can be combined with a carrier material to produce a single dosage form will generally be that amount of the compound which produces a desired skin, hair, and/or eye lightening effect. Wetting agents, emulsifiers and lubricants, such as sodium lauryl sulfate and magnesium stearate, as well as coloring agents, release agents, and perfuming agents, preservatives and antioxidants can also be present in the compositions.

In some embodiments, the pharmaceutically acceptable topical formulations as contemplated herein comprise at least a compound as described herein and a penetration enhancing agent. The choice of topical formulation will depend on several factors, including the condition to be treated, the physicochemical characteristics of the administered compound and other excipients present, their stability in the formulation, available manufacturing equipment, and costs constraints. As used herein the term “penetration enhancing agent” means an agent capable of transporting a pharmacologically active compound through the stratum comeum and into the epidermis or dermis, preferably, with little or no systemic absorption. In certain exemplary embodiments, penetration agents for use with the compositions described herein include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), dimethyl sulfoxide (DMSO) and N-methyl pyrrolidone. In some embodiments, the formulation comprises dimethyl sulfoxide (DMSO).

Various formulations comprising the skin, hair, and/or eye lightening agents can be prepared according to any method known to the art for the manufacture of pharmaceuticals. A formulation can be admixed with nontoxic pharmaceutically acceptable excipients which are suitable for manufacture. Formulations may comprise one or more diluents, emulsifiers, preservatives, buffers, excipients, etc. and may be provided in such forms as powders, emulsions, lyophilized powders, sprays, creams, lotions, controlled release formulations, gels, on patches, in implants. etc.

Aqueous suspensions can contain a skin, hair, and/or eye lightening agent described herein in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanthin and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate and one or more coloring agents. Formulations can be adjusted for osmolarity.

In some embodiments, oil-based pharmaceuticals or compositions are used for administration. Oil-based suspensions can be formulated by suspending an active agent in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. As an example of an injectable oil vehicle, see Minto (1997) J. Pharmacol. Exp. Ther. 281:93-102.

Compositions useful herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. In alternative embodiments, injectable oil-in-water emulsions described herein comprise a paraffin oil, a sorbitan monooleate, an ethoxylated sorbitan monooleate and/or an ethoxylated sorbitan trioleate.

In some embodiments, the composition is a pharmaceutical or cosmetic composition used in the treatment of pigmentation disorders (e.g., post inflammatory hyperpigmentation, lentigines, lafe au lait macules, ephelides, seborrhoic keratosis, nevi, melasma, incontinetia pigmenti, dowling-degos-syndrome, and metabolic and secondary hyperpigmentation). The pharmaceutical compositions should provide a sufficient quantity of active agent to effectively treat, prevent (reduce risk of), or ameliorate conditions, diseases or symptoms. The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods described herein are correct and appropriate.

### Exemplary Embodiments

The following exemplary embodiments are further provided herein:

A method of decreasing pigmentation in the skin of a subject, said method comprising providing a composition comprising at least one of Leflunomide, Cpd A, Cpd B, Mi111, Naproxol. Candesartan, Hextidine, Elaidylphosphocholine, or combinations thereof, to the skin of a subject in an amount sufficient to decrease pigmentation.

In some embodiments, the subject has a pigmentation disorder, wherein pigmentation in the subject is increased compared to a reference.

In some embodiments, the disorder is characterized by increased pigmentation caused by post inflammatory hyperpigmentation, lentigines, lafe au lait macules, ephelides, seborrhoic keratosis, nevi, melasma, incontinetia pigmenti, dowling-degos-syndrome, and metabolic and secondary hyperpigmentation.

A method of decreasing UVB and/or UVA-induced pigmentation in the skin of a subject, said method comprising providing a composition comprising at least one of Leflunomide, Cpd A, Cpd B, Mi11, Naproxol, Candesartan, Hextidine, Elaidylphosphocholine, or combinations thereof, to the skin of a subject following UVB and/or UVA exposure.

A method of decreasing pigmentation in the hair of a subject, said method comprising providing a composition comprising at least one of Leflunomide, Cpd A. Cpd B, Mi11, Naproxol, Candesartan, Hextidine, Elaidylphosphocholine, or combinations thereof, to the hair of a subject in an amount sufficient to decrease pigmentation.

A method of decreasing pigmentation in the eye of a subject, said method comprising providing a composition comprising at least one of Leflunomide, Cpd A, Cpd B, Mi111, Naproxol, Candesartan, Hextidine, Elaidylphosphocholine, or combinations thereof, to the hair of a subject in an amount sufficient to decrease pigmentation.

A method of visible light-induced pigmentation in the skin of a subject, said method comprising providing a composition comprising at least one of Leflunomide, Cpd A, Cpd B, Mi11, Naproxol, Candesartan, Hextidine, Elaidylphosphocholine, or combinations thereof, to the skin of a subject following visible light exposure.

### EXAMPLES

The present invention is additionally described by way of the following illustrative, non-limiting Examples that provide a better understanding of the present invention and of its many advantages.

### Methods

The following materials and methods were used in the examples, below, unless otherwise indicated.

REAGENT or RESOURCE

| | | |
|---|---|---|
| KEY RESOURCES TABLE | SOURCE | IDENTIFIER |

Antibodies

| | | |
|---|---|---|
| anti-MITF monoclonal antibody C5 | Made in the lab | (King et al., 1999) |
| | of Dr. David E. | |
| | Fisher | |
| Mouse monoclonal anti-tyrosinase antibody, | Sigma-Adirich | Cat# 05-647; RRID: |
| clone T311 | | RRID: AB_309873 |
| Donkey anti-rabbit IgG-HRP | ThermoFisher | Cat# 45-000-683; |
| | Scientific | RRID: AB_2721111 |
| Amersham ECL mouse IgG, HRP | ThermoFisher | Cat#45000680; RRID: AB_2721110 |
| | Scientific | |
| Monoclonal anti-β-actin-peroxidase | Sigma Aldrich | Cat# A3854; RRID: AB_262011 |
| Alexa Fluor 555 goat anti-rabbit IgG (H + L), | ThermoFisher | Cat# A-21428; RRID: AB_2535849 |
| secondary antibody | Scientific | |
| Alexa Fluor 647 goat anti mouse IgG (G + L); | ThermoFisher | Cat# A-21236; RRID: AB_2535805 |
| fluorescence conjugated secondary antibody | Scientific | |
| Alexa Fluor 594 F(ab)2 fragment of goat | ThermoFisher | Cat# A-11072; RRID: AB_2534116 |
| anti-rabbit IgG (G + L); fluorescence | Scientific | |
| conjugated secondary antibody | | |
| Alexa Fluor 488-conjugated donkey anti-mouse | ThermoFisher | Cat# A-21202; RRID: AB_141607 |
| secondary antibodies | Scientific | |
| Anti-Cyclobutane Pyrimidine Dimers (CPDs) | Cosmo Bio USA | Cat# CAC-NM-DND-001; |
| mAb antibody (Clone TDM-2) | | RRID: AB_1962813 |
| Rabbit anti-gamma-H2AX (P-ser139) polyclonal | NOVUS | Cat# NB100-384; |
| antibody | Biologicals | RRID: AB_10002815 |
| Mouse monoclonal anti-Mitofusin 2 antibody | Abcam | Cat# ab56889; RRID: AB_2142629 |
| [6A8] | | |
| Rabbit polyclonal anti TRP2/DCT antibody | Abcam | Cat# ab74073; RRID: AB_1524517 |
| Mouse monoclonal anti-NNT antibody [8B4BB10] | Abcam | Cat# ab110352; RRID: AB_10887748 |
| Rabbit anti-NNT (C-terminal) polyclonal | Abcam | Cat# ab214212; RRID: AB_2889980 |
| antibody | | |
| Mouse anti-8-oxo-dG monoclonal antibody | Trevigen | Cat# 4354-MC-050; |
| | | RRID: AB_1857195 |
| IDH1 (D2H1) Rabbit mAb | Cell Signaling | Cat# 8137; RRID: AB_10950504 |
| | Technology | |
| Mouse monoclonal p53 antibody [PAb 240] | Abcam | Cat# ab26; RRID: AB_303198 |
| Rabbit monoclonal TRP1 antibody [EPR21960] | Abcam | Cat# ab235447; RRID: AB_2889980 |
| Mouse monoclonal antibody PmeI17 (E-7) | Santa Cruz | Cat# sc-377325; RRID: AB_2889982 |
| | Biotechnology | |
| LC3B (D11) rabbit monoclonal antibody | Cell Signaling | Cat#38668S RRID: AB_2137707 |
| | Technology | |

Biological Samples

| | | |
|---|---|---|
| Full thickness human breast and abdominal | Massachusetts | IRB# 2013P000093 |
| skin explants | General Hospital | |
| Paraffin-embedded formalin fixed slides, | Massachusetts | IRB# 2013P000093 |
| prepared from breast and abdominal biopsy | General Hospital | |
| samples | | |
| Human skin samples for genome wide | Massachusetts | IRB# 2013P000093 |
| association study (GWAS) | General Hospital | |
| | or the | |
| | Cooperative | |
| | Human Tissue | |
| | Network | |

Chemicals, Peptides, and Recombinant Proteins

| | | |
|---|---|---|
| 3-isobutyl-1-methylxanthine (IBMX) | Sigma-Aldrich | Cat# I5879 |
| 12-O-tetradecanoylphorbol-13-acetate (TPA) | Sigma-Aldrich | Cat# 16561-29-8 |
| Ham′s F10 | Thermo Fisher | Cat# MT10070CV |
| | Scientific | |
| N6,2′-O-Dibutyryladenosine 3′,5′-cyclic | Sigma-Aldrich | Cat# D0627 |
| monophosphate sodium salt | | |
| Penicillin-Streptomycin | Thermo Fisher | Cat# 15140163 |
| | Scientific | |
| Na3VO4 | Sigma-Aldrich | Cat# 450243 |
| Medium 254 | Life | Cat# M254500 |
| | Technologies | |
| 0.05% Trypsin-EDTA w/ phenol red | Life | Cat# 25300120 |
| | Technologies | |
| Human Melanocyte Growth Supplement (HMGS) | Life | Cat# S0025 |
| | Technologies | |
| Bovine Serum Albumin | Sigma | Cat#A7030 |
| Goat serum | Sigma-Aldrich | Cat# G9023 |
| RPMI (Roswell Park Memorial Institute 1640 | Life | Cat# 11875119 |
| Medium) | Technologies | |
| RIPA lysis buffer | Sigma-Aldrich | Cat# R0278 |
| FetalPlex Animal Serum Complex | Gemini Bio- | Cat# 100-602 |
| | Products | |
| Western Lightning Plus-ECL | PerkinElmer | Cat# NEL105001EA |
| Non-fat milk powder | Boston | Cat# P-1400 |
| | BioProducts | |
| Protein Block | Agilent | Cat# X090930-2 |
| Antibody Diluent | DAKO | Cat# S3022 |
| VECTASHIELD ® HardSet ™ Antifade Mounting | Vector | Cat# H-1500 |
| Medium with DAPI | Laboratories | |
| synthetic melanin | Sigma Aldrich | Cat# M8631 |
| N,N-Dicyclohexylcarbodiimide [DCC] | Sigma Aldrich | Cat# D80002 |
| 2,3-Butanedione [2,3BD] | Sigma Aldrich | Cat# B85307 |
| Palmitoyl coenzyme A lithium salt | Sigma Aldrich | Cat# #P9716 |
| cycloheximide (CHX) | Sigma Aldrich | Cat# C7698 |
| NADPH | Sigma Aldrich | Cat# N7505 |
| N-Acetyl-L-cysteine (NAC) | Sigma Aldrich | Cat# A7250 |
| Mito TEMPO | ThermoFisher | Cat# 501872447 |
| | Scientific | |
| Hydrogen peroxide solution | Sigma Aldrich | Cat# 216763 |
| SYBR FAST qPCR master mix | Kapa Biosystems | Cat# KK4600; |
| Protease and Phosphatase Inhibitor | ThermoFisher | Cat# PI78445 |
| | Scientific | |
| Westem Lightning Plus-ECL, Enhanced | Perkin Elmer | Cat# NEL105001EA |
| Chemiluminescence Substrate | | |
| MitoSOX Red | ThermoFisher | Cat# M36008 |
| | Scientific | |
| CM-H2DCFDA | ThermoFisher | Cat# C6827 |
| | Scientific | |
| NucBlue | ThermoFisher | Cat# R37605 |
| | Scientific | |
| Karnovsky′s fixative (2% paraformaldehyde/ | Prepared in the | N/A |
| 2.5% glutaraldehyde in 0.1 M sodium | lab of Dr. David | |
| cacodylate buffer, pH 7.4) | E. Fisher | |
| Polybrene | Sigma-Aldrich | Cat# TR-1003 |
| Paraformaldehyde 16% | ThermoFisher | Cat# 50980487 |
| | Scientific | |
| Ethanol | Thermo Fisher | Cat# 04355226 |
| | Scientific | |
| Triton X-100 | Sigma Aldrich | Cat# T8787 |
| TWEEN ® 20 | Sigma Aldrich | Cat# P7949 |
| Forskolin from Coleus forskohlii, ≥98% | Sigma Aldrich | Cat# F6886 |
| Lipofectamine RNAiMAX Transfection Reagent | Life | Cat# 13778150 |
| | Technologies | |
| IQ5 High-fidelity DNA Polymerase | New England | Cat# M0491S |
| | Biolabs | |

Critical Commercial Assays

| | | |
|---|---|---|
| Direct cAMP ELISA Kit | Enzo Life | Cat# ADI-901-066 |
| | Sciences | |
| GSH/GSSG-Glo Assay | Promega | Cat# V6611 |
| CellTiter-Glo Luminescent Cell Viability | Promega | Cat# G7570 |
| Assay | | |
| Pierce BCA protein assay | ThermoFisher | Cat# 23225 |
| | Scientific | |
| KAPA Library Quantification Kits | Roche | Cat# 7960140001 |
| MiSeq Reagent Kits v2 (300 cycles) | Illumina | Cat# MS-102-2002 |
| MaxBlock Autofluorescence Reducing Reagent | MaxVision | Cat# MB-L |
| Kit | Biosciences | |
| Fontana-Masson Stain Kit (Melanin Stain) | Abcam | Cat# ab150669 |
| Dual Reporter System | GeneCopoeia | Cat# HPRM39435-LvPM02 |
| Secrete-Pair Gaussia Luciferase Assay Kit | GeneCopoeia | Cat# LF062 |
| NADP/NADPH-Glo Assay | Promega | Cat# G9082 |
| QUANTI-Blue ™ Solution | InvivoGen | Cat# rep-qbs |
| RNeasy Plus Mini Kit | Qiagen | Cat# 74136 |

Publicly Available Data

| | | |
|---|---|---|
| Ensembl database information for promoter | Ensembl | ensembl.org/Homo_sapiens/ |
| ENSR00000180214 of the NNT gene | | Regulation/Summary?fdb=funcgen; |
| | | r=5:43600000- |
| GTEx expression database | GTEx | gtexportal.org |
| GWAS summary statistics from the CANDELA | GWAS Central | gwascentral.org/study/HGVST3308 |
| cohort | | |
| GWAS summary statistics from the UK Biobank | | cnsgenomics.com/software/gcta/# |
| cohort | | DataResource |

Experimental Models: Cell Lines

| | | |
|---|---|---|
| Primary human melanocytes (isolated from | Massachusetts | IRB# 2013P000093 |
| neonatal foreskins) | General Hospital | |
| Human melanoma cell line UACC257 | National Cancer | |
| | Institute | |
| | Division | |
| | of Cancer | |
| | Treatment and | |
| | Diagnosis | |
| | (DCTD) Tumor | |
| | Repository | |
| Human melanoma cell line SK-MEL-30 | Memorial Sloan | mskcc.org/ |
| | Kettering Cancer | |
| | Center | |
| Mouse Melan-A cell line | Wellcome Trust | Bennett et al., 1987 |
| | Functional | |
| | Genomics Cell | |
| | Bank | |
| Primary human keratinocytes isolated from | Massachusetts | IRB# 2013P000093 |
| discarded surgical human skin tissue (e.g., | General Hospital | |
| foreskins) | | |
| Primary human fibroblasts isolated from | Massachusetts | IRB# 2013P000093 |
| discarded surgical human skin tissue (e.g., | General Hospital | |
| foreskins) | | |
| Lenti-X ™ 293T cells | Clontech | Cat# 632180 |

Experimental Models: Organisms/Strains

| | | |
|---|---|---|
| C57BL/6J mice | Jackson | Stock No: 000664 |
| | Laboratory | |
| C57BL/6NJ mice | Jackson | Stock No: 005304 |
| | Laboratory | |
| Casper zebrafish (mitfa−/−; roy−/−) embryos | Laboratory of Dr. | |
| | Leonard I. Zon | |

Oligonucleotides

| | | |
|---|---|---|
| nhelkozakHAhNNT_f1: forward, | eurofins | For PLMJ1-HA-NNT |
| 5′ | Genomics | |
| ctagctagcCCGCCACCATGTACCCATACGATGTTOCA | | |
| GATTACGCTGCAAACCTATTGAAAACAGTGGTGACT | | |
| G-3′ (SEQ ID NO: 1) | | |
| hNNTnheI_r1: reverse, | eurofins | For PLMJ1-HA-NNT |
| 5′- | Genomics | |
| ctagctagcTTACTTCTGATAGGATTCTCTAACTTTCGC- | | |
| 3′ (SEQ ID NO: 2) | | |
| nheikozakhMFN2_f1: forward, | eurofins | For PLMJ-HA MFN2 |
| 5′- | Genomics | |
| ctagctagcGCCACCATGTCCCTGCTCTTCTCTCGATG | | |
| C-3′ (SEQ ID NO: 3) | | |
| hMFN2(HA)nheI_r1: reverse, | eurofins | For PLMJ1-HA MFN2 |
| 5′-ctagctagcTTAGGATCCAGCAGCGTAATCTGGAAC- | Genomics | |
| 3′ (SEQ ID NO: 4) | | |
| RT-Primers for NNT, IDH1, MFN2, TYRP1, | | |
| DCT/TRP2, MITF, POMC, PPARGC1A, Tyrosinase: | | |
| See Table below | | |

Recombinant DNA

| | | |
|---|---|---|
| CRISPR MiniCoopR-U6:gRNA-mitfa:Cas9 plasmid | Addgene | Cloned from Addgene plasmid ID |
| | | 118840 |
| pMiniCoopR-mitfa:NNT expression plasmid | Addgene | Cloned from Addgene plasmid ID |
| | | 118850 |
| PLMJ1-NNT-HA | This manuscript | Based on Addgene plasmid, # 19319 |
| PLMJ1-EGFP plasmid | Laboratory of Dr. | Addgene plasmid, # 19319; |
| | David Sabatini | n2t.net/addgene: 19319; |
| | | RRID: Addgene_19319, (Sancak et |
| | | al., 2008) |
| pcDNA3.1 Mfn2HA | Laboratory of Dr. | Addgene plasmid, # 139192; |
| | Allan Weissman | n2t.net/addgene: 139192; |
| | | RRID: Addgene_139192 ((Leboucher |
| | | et al., 2012) |
| PLMJ1-MFN2-HA | This manuscript | Based on Addgene plasmid, # |
| | | 139192 and Addgene plasmid, # |
| | | 19319 |

Software and Algorithms

| | | |
|---|---|---|
| GraphPad Prism 8.4.3 (471) | GraphPad | graphpad.com/scientific- |
| | | software/prism/ |
| ImageJ (v1.8.0) | National | imagej.nih.gov/ij/ |
| | Institutes of | |
| | Health | |
| FIJI software for pixel-based color | FIJI | imagej.net/Fiji |
| quantification | | |
| Off-target prediction software (for design | (Bae et al., | rgenome.net/cas-offinder/ |
| of guide RNAs) | 2014) | |
| On-target prediction software (for design | (Moreno-Mateos | crisprscan.org/ |
| of guide RNAs); CRISPRscan and CHOPCHOP | et al., 2015) | chopchop.cbu.uib.no/ |
| | (Labun et al., | |
| | 2019) | |
| Axiovision REL 4.7 software | Zeiss | N/A |
| CRISPResso2 software (for genome editing) | Kendell Clement | Nat Biotechnol. 2019 March; |
| | et al | 37(3): 224-226. |
| MACH software | | csg.sph.umich.edu/abecasis/mach/ |
| | | index.html |
| GCTA program | Yang et al., | cnsgenomics.com/software/gcta/ |
| | 2011 | |
| PLINK program | | cog-genomics.org/plink/1.9/ |
| BioRender | | BioRender.com |
| Fiji | Saalfeld, S., | imagej.net/Fiji |
| | Schmid, B., et | |
| | al. (2012) | |
| NDP.view2 Viewing software | HAMAMATSU | hamamatsu.com/us/en/product/ |
| | | type/U12388-01/index.html |

Other

| | | |
|---|---|---|
| SiGENOME Human MITF siRNA SMARTpool | Dharmacon | Cat# M-008674-00-0005 |
| ON-TARGETplus Human IDH1 siRNA SMARTpool | Horizon Inspired | Cat# L-008294-01-0005 |
| | Cell Solutions | |
| ON-TARGETplus Human MFN2 siRNA SMARTpool | Horizon Inspired | Cat# L-012961-00-0005 |
| | Cell Solutions | |
| ON-TARGETplus Human NNT siRNA SMARTpool | Horizon Inspired | Cat# L-009809-00-0005 |
| | Cell Solutions | |
| ON-TARGETplus Human PPRGC1A siRNA SMARTpool | Horizon Inspired | Cat# L-005111-00-0005 |
| | Cell Solutions | |
| ON-TARGETplus Human Tyrosinase siRNA | Horizon Inspired | Cat# L-012555-00-0005 |
| SMARTpool | Cell Solutions | |
| ON-TARGETplus non-targeting siRNA control | Horizon Inspired | Cat# D-001810-10-05 |
| pool | Cell Solutions | |
| 4-15% Criterion TGX Precast Midi Protein | Bio-Rad | Cat# 5671084 |
| gels | Laboratories | |
| Chamber slides | ThermoFisher | Cat# 125657 |
| | Scientific | |

### Experimental Models and Subjects

### Mice

All mice were bred on a heterozygous MiWhite background (Mitf white) (Steingrimsson et al., 2004). C57BU6J mice (Jackson Laboratory, Stock No: 000664) displaying a 5-exon deletion in the Nnt gene resulting in a homozygous loss were compared to Nnt wild type C57BU6NJ mice (Jackson Laboratory, Stock No: 005304). All mice were matched by gender and age (female, 6 weeks old). Mice were genotyped according to the protocol obtained from Jackson Laboratory (protocol 26539: Standard PCR Assay—Nnt<C57BL/6J>, Version 2.2).

### Zebrafish

### Overexpression of Human NNT in Zebransh

The human NNT gene was cloned into the MiniCoopR expression plasmid to allow melanocyte-specific overexpression of NNT (Ceol et al., 2011). The mcr:NNT plasmid was injected into Tubingen zebrafish embryos at the single cell stage and incorporated into the genome through the use of Tol2 transgenesis. Larvae were raised for 5 days and then at least three images were obtained and quantified using a Nikon SMZ18 Stereomicroscope. At least 5 zebrafish embryos of each group were analyzed after 5 days using the TinEye software enabling pixel-based color quantification.

### Deletion of Zebrafish Nnt Gene

SpCas9 guide RNAs (gRNAs) were designed to target the first two exons of the zebrafish nnt gene using on-target and off-target prediction software. gRNA expression plasmids were constructed by cloning oligonucleotides (Integrated DNA Technologies) into BseRl-digested pMiniCoopR-U6:gRNA-mitfa:Cas9 (Addgene plasmid ID 118840) (Ablain et al., Dev Cell 2015). A control CRISPR MiniCoopR plasmid was generated by cloning a scrambled gRNA into the CRISPR MiniCoopR vector. The CRISPR MiniCoopR plasmid contains an mitf mini-gene alongside mitfa:Cas9 and U6:gRNA. Casper zebrafish (mitfa−/−; roy-I-) embryos (Ablain et al., 2015) were injected at the single cell stage with plasmid DNA, which gets incorporated into the genome though Tol2 transgenesis. This results in the rescue of melanocytes via the mitfa minigene and melanocyte-specific knockout of nnt. Larvae were raised for 4 days and imaged using a Nikon SMZ18 Stereomicroscope. DNA was extracted from the embryos at 4 days post fertilization using the Hot Shot method (Truett, et al, BioTechniques 2000), for analysis of genome editing. The efficiency of genome modification by SpCas9 was determined by next-generation sequencing using a 2-step PCR-based Illumina library construction method, as previously described (Walton et al., 2020). Briefly, genomic loci were amplified from gDNA extracted from pooled samples of 8-10 zebrafish embryos using 05 High-fidelity DNA Polymerase (New England Biolabs, #M0491S). PCR products were purified using paramagnetic beads prepared as previously described (Rohland and Reich, 2012) (Kleinstiver et al., 2019). Approximately 20 ng of purified PCR product was used as template for a second PCR to add Illumina barcodes and adapter sequences using Q5. PCR products were purified prior to quantification via capillary electrophoresis (Qiagen QlAxcel), followed by normalization and pooling. Final libraries were quantified by qPCR using a KAPA Library Quantification Kit (Roche, #7960140001) and sequenced on a MiSeq sequencer using a 300-cycle v2 kit (Illumina, #MS-102-2002). Genome editing activities were determined from the sequencing data using CRISPResso2 (Clement et al., 2019) with default parameters.
Chemical treatment of Zebransh

Wildtype Tübingen zebrafish were placed in a 24 well plate at 72 hours post-fertilization, with 10 larvae per well for a total twenty larvae per condition. Larvae were treated for 24 hours with either 2,3BD (1 μM, 10 μM, 100 μM, 1 mM; Sigma Aldrich, #B85307), DCC (1 μM, 10 μM, 50 μM, 100 μM; Sigma Aldrich, #D80002), or DMSO (1:500) in E3 embryo medium. At 4 days post fertilization, larvae were imaged using a Nikon SMZ18 Stereomicroscope. Melanocytes from at least 5 zebrafish embryos of each group for each experiment were analyzed using the FIJI software enabling pixel-based color quantification.

### Human Skin Explants

Skin samples considered surgical waste were obtained de-identified from healthy donors (IRB #2013P000093) undergoing reconstructive surgery, according to institutional regulations. Full thickness human abdominal skin explants were cultured in petri dishes with a solid phase and liquid phase phenol red free DMEM medium containing 20% penicillin/streptomycin/glutamine, 5% fungizone (Gibco), and 10% fetal bovine serum. Explants were treated with vehicle (DMSO), 2,3BD (50 mM, 1 M, or 11 M;) or DCC (50 mM) as indicated in the figure legends. Compounds were applied strictly on top of the explants, making sure no drip occurred into the underlying media. For UV irradiation experiments, a UV lamp (UV Products) was used at 1000 mJ/cm² UVB.

### Cell Lines

Primary human melanocytes were isolated from normal discarded foreskins and were established in TIVA medium as described previously (Khaled et al., 2010) or in Medium 254 (Life Technologies, #M254500) (Allouche et al., 2015). Human melanoma cell line UACC257 (sex unspecified) was obtained from the National Cancer Institute (NCI), Frederick Cancer Division of Cancer Treatment and Diagnosis (DCTD) Tumor Cell Line Repository. SK-MEL-30 (male) human melanoma cell line was from Memorial Sloan Kettering Cancer Center. Both melanoma cell lines have been authenticated by our lab using ATCC's STR profiling service. UACC257 and SK-MEL-30 cells were cultured in DMEM and RPMI medium (Life Technologies, #11875119) respectively, supplemented with 10% fetal bovine serum and 1% penicillin/streptomycin/L-glutamine in a humidified atmosphere of 95% air and 5% CO₂ at 37° C.

Murine Melan-A (Bennett et al., 1987) cells were obtained from the Wellcome Trust Functional Genomics Cell Bank. Melan-A cells were grown in RPMI 1640 supplemented with 10% FBS or FetalPlex (Gemini Bio-Products, #100-602), 100,000 U/L penicillin, 100 mg/L streptomycin sulphate, 100× Glutamax, and 200 nM TPA.

Primary human keratinocytes were cultured in EpiLife® medium supplemented with human keratinocyte growth supplement (HKGS, ThermoFisher Scientific). Primary human fibroblasts were cultured in medium 106 supplemented with low serum growth supplement (LSGS, ThermoFisher Scientific). 10⁶ and 104 cells were plated per well of 6-well and 96-well plates, respectively. Drugs indicated in the figure legends were dissolved in DMSO and added 1:1000 to the culture media for 24 h at the concentrations indicated.

### Method Details

sIRNA transfection: A single treatment of 10 nmol/L of siRNA was delivered to a 60% confluent culture by transfection with Lipofectamine RNAiMAX (Life Technologies, #13778150) according to the manufacturer's recommendations. After 48-72 h of transfection, total RNA or protein was harvested.

Plasmid overexpression: Human NNT fused to a haemagglutinin (HA)-tag at the N-terminus was amplified from pEGFP-C1-hNNT (primer sequences are in the Key Resources Table) and was subcloned into the Nhel restriction site of pLMJ1-EGFP [a gift from David Sabatini, Addgene plasmid #19319, n2t.net/addgene:19319, RRID:Addgene_19319 (Sancak et al., 2008)] using Nhel (New England Biolabs, R3131S).

For human MFN2 overexpression, human MFN2 fused to three HA tags at the C-terminus was amplified from pcDNA3.1 Mfn2HA (a gift from Allan Weissman, Addgene plasmid 139192, n2t.net/addgene:139192, RRID:Addgene_139192 (Leboucher et al., 2012) (primer sequences are in the Key Resources Table) and was subcloned into the Nhel restriction site of pLJM1-EGFP using Nhel (New England Biolabs, #R3131S).

FLAG-tagged human NNT cDNA (NNT-FLAG) was purchased from Origene (RC224002). The NNT-FLAG cassette was re-cloned into pLJM1-EGFP (Addgene #19319) following Nhel and EcoRI digestion.

Lentivirus generation and infection: Lentivirus was generated in Lenti-X™ 293T cells (Clontech, #632180). The Lenti-X cells were transfected using 250 ng pMD2.G, 1250 ng psPAX2, and 1250 ng lentiviral expression vector in the presence of PEI (MW:25K). For infection with lentivirus, 0.1-1 ml of lentivirus-containing medium was used in the presence of 8 μg/ml polybrene (Sigma. #TR-1003). Selection with puromycin (10 μg/ml) was performed the day after infection.

In vitro culture with NNT inhibitors: 2,3-Butanedione 97% (2,3 BD) (Sigma Aldrich, #B85307) (1 μM, 10 μM, 100 μM, 2 mM), N,N-Dicyclohexylcarbodiimide (DCC) (Sigma Aldrich, #D80002) (1 mM, 2 mM, 10 mM), and Palmitoyl coenzyme A lithium salt (Sigma Aldrich, #P9716) (10 μM, 2 mM) were reconstituted with DMSO (American Type Culture Collection, 4-X).

Immunoblotting: Whole-cell protein lysates were prepared using RIPA lysis buffer (Sigma-Aldrich, #R0278) supplemented with Protease and Phosphatase Inhibitor (ThermoFisher Scientific, #P178445). Protein concentrations were quantified using the Pierce BCA protein assay (ThermoFisher Scientific, #23225). Immunoblotting was performed by standard techniques using 4-15% Criterion TGX Precast Midi Protein gels (Bio-Rad Laboratories, #5671084) and transferring to 0.2 μm nitrocellulose membranes (Bio-Rad Laboratories, #1620112). Membranes were blocked with 5% non-fat milk (Boston BioProducts, #P-1400) in PBS containing 0.1% Tween 100 and incubated with one of the following primary antibodies at the indicated dilution (antibody sources are in the Key Resources Table): 1:20 dilution of anti-MITF monoclonal antibody CS, 1:1,000 dilution of anti-Tyrosinase clone T311, 1:1,000 dilution of anti-Mitofusin-2 antibody [6A8], 1:500 dilution of TRP2/DCT antibody, 1:1,000 dilution of anti-NNT antibody [8B4BB10], 1:1,000 dilution of anti-IDH1 (D2H1) antibody, 1:1,000 dilution of p53 antibody [PAb 240], 1:1,000 dilution of TYRP1 antibody [EPR21960], 1:1,000 dilution of mouse monoclonal antibody Pmel17 (E-7), or 1:1,000 dilution of LC3B (D11) rabbit monoclonal antibody. Incubation with the appropriate secondary antibody followed, either a 1:5,000 dilution of donkey anti-Rabbit IgG-HRP or a 1:3,000 dilution of Amersham ECL mouse IgG, HRP.

To verify equal loading of samples, membranes were re-probed with a 1:20,000 dilution of monoclonal anti-β-actin-peroxidase (Sigma Aldrich, #A3854). Protein bands were visualized using Western Lightning Plus ECL (PerkinElmer, #NEL105001EA) and quantified using ImageJ software (NIH).

RNA purification and quantitative RT-PCR: Total RNA was isolated from cultured primary melanocytes or melanoma cells at the indicated time points, using the RNeasy Plus Mini Kit (Qiagen, #74136). mRNA expression was determined using intron-spanning primers with SYBR FAST qPCR master mix (Kapa Biosystems, #KK4600). Expression values were calculated using the comparative threshold cycle method (2^{-ΔΔCt}) and normalized to human RPL11 mRNA. The primers used for quantitative RT-PCR (eurofins Genomics) and are listed below.

SEQ ID NO:

| Primer | Sequence | SEQ ID |
|---|---|---|
| Human RPL11: forward | 5′-GTTGGGGAGAGTGGAGACAG-3′ | 5 |
| Human RPL11: reverse | 5′-TGCCAAAGGATCTGACAGTG-3′ | 6 |
| Human M isoform MITF: forward | 5′-CATTGTTATGCTGGAAATGCTAGAA-3′ | 7 |
| Human M isoform MITF: reverse | 5′-GGCTTGCTGTATGTGGTACTTGG-3′ | 8 |
| Human Tyrosinase: forward | 5′-ACCGGGAATCCTACATGGTTCCTT-3′ | 9 |
| Human Tyrosinase: reverse | 5′-ATGACCAGATCCGACTCGCTTGTT-3′ | 10 |
| Human NNT: forward | 5′-AGCTCAATACCCCATTGCTG-3′ | 11 |
| Human NNT: reverse | 5′-CACATTAAGCTGACCAGGCA-3′ | 12 |
| Human IDH1: forward | 5′-GTC GTCATGCTTATGGGG AT-3′ | 13 |
| Human IDH1 reverse | 5′-CTT TTGGGTTCCGTCACT TG-3′ | 14 |
| Huma MFN2: forward | 5′-CTG CTA AGG AGGTGCTCA A-3′ | 15 |
| Human MFN2: reverse | 5′-TCC TCA CTTGAAAGC CTT CTG C-3′ | 16 |
| Human PPARGC1A: forward | 5′-CTG CTA GCA AGTTTG CCT CA-3′ | 17 |
| Human PPARGC1A: reverse | 5′-AGTGGTGCAGTGACCAATCA-3′ | 18 |
| Human POMC: forward | 5′-AAGAGGCTAGAGGTCATCAG-3′ | 19 |
| Human POMC: reverse | 5′-AGAACGCCATCATCAAGAAC-3′ | 20 |
| Human TYRP1 forward | 5′-CCAGTCACCAACACAGAAATG-3′ | 21 |
| Human TYRP1 reverse | 5′-GTGCAACCAGTAACAAAGCG-3′ | 22 |
| Human TRP2/DCT forward | 5′-TTCTCACATCAAGGACCTGC-3′ | 23 |
| Human TRP2/DCT reverse | 5′-ACACATCACACTCGTTCCTC-3′ | 24 |

Cycloheximide chase assay: 72 h after siRNA transfection (sicontrol or siNNT), UACC257 melanoma cells were treated with a protein synthesis inhibitor, cyclohexamide (CHX, Sigma Aldrich #C7698, 50 μg/ml), for the indicated times and then immediately subjected to immunoblotting for tyrosinase protein expression. The expression of tyrosinase was quantified using ImageJ software based on band intensities and normalized to the intensities of the corresponding β-actin bands. The normalized tyrosinase expression was then defined as relative tyrosinase expression by setting the mean values at t=0 in each experimental group to 1.0. In the ROS rescue experiments, siRNA-containing medium was replaced with fresh culture medium containing either N-acetyl-L-cysteine (NAC; Sigma Aldrich #A7250, 5 mM), β-nicontinamide adenine dinucleotide 2′-phosphate (NADPH; Sigma Aldrich #N7505, 0.1 mM), MitoTEMPO (ThermoFisher #501872447, 20 mM) or control vehicle (DMSO or TrisHCl respectively) 24 h after siRNA transfection. The siRNA-transfected cells were cultured for an additional 48 h in the presence of these agents and then examined by the CHX chase assay as described above. pLJM-1-EGFP or pLJM1-NNT/FLAG was introduced into UACC257 cells using Lipofectamine 3000, 48 after transfection, the transfection medium was replaced with fresh medium containing DMSO or 10 μM MG132 (Sigma Aldrich #M8699) and pre-incubated for 6 h. Then. CHX was added to assess tyrosinase protein stability as described above.

Melanin quantification: Equal numbers of cells were plated in 6-well plates. The cells were then harvested 72-96 hours post siRNA or NNT inhibitors compounds, as indicated in the legends, pelleted, washed in PBS and counted. 10⁶ cells were used for measurement of protein concentration with the Pierce BCA protein assay (Thermo Fisher Scientific, #23225) and 10⁶ cells were resuspended in 60 μl of 1 N NaOH solution and incubated at 60° C. for 2 h or until the melanin was completely dissolved. After cooling down to room temperature, samples were centrifuged at 500×g for 10 min and the supernatants were loaded onto a 96-well plate. The melanin content was determined by measuring the absorbance at 405 nm on an Envision plate reader, compared with a melanin standard (0 to 50 μg/ml; Sigma Aldrich, #M8631). Melanin content was expressed as micrograms per milligram of protein.

Eumelanin and pheomelanin analysis: Lyophilized cells (10⁶) from mouse fur or human abdominal full thickness skin explants were ultrasonicated in 400 mL of water and fur samples were homogenized at a concentration of 10 mg/mL in water in a Ten-Broeck homogenizer. Aliquots of 100 mL were subjected to alkaline hydrogen peroxide oxidation to yield the eumelanin marker pyrrole-2,3,5-tricarboxylic acid (PTCA) (Ito et al., 2011), or to hydroiodic acid (HI) hydrolysis to yield the pheomelanin marker 4-amino-3-hydroxyphenylalanine (4-AHP) (Wakamatsu et al., 2002), then the samples were analyzed by HPLC. Amounts of each marker are reported as ng of marker per 10⁶ cells or mg fur. Pheomelanin and eumelanin contents were calculated by multiplying the 4-AHP and PTCA contents by factors of 7 and 25, respectively (d'Ischia et al., 2013).

Skin colorimeter measurements: Skin reflectance measurements were made using a CR-400 Colorimeter (Minolta Corporation, Japan). Before each measurement, the instrument was calibrated against the white standard background provided by the manufacturer. The degree of melanization (darkness) is defined as the colorimetric measurement on the *L axis (luminance, ranging from completely white to completely black) of the Centre Internationale d'Eclairage (CIE) L*a*b* color system (Park et al., 1999). Each data point is the mean of measurements performed in technical triplicate (three different locations within the same ear).

Determination of intracellular cAMP content: Cyclic adenosine monophosphate (cAMP) was measured directly using an enzyme-linked immunosorbent assay (ELISA) (Enzo Life Sciences, #ADI-901-066). cAMP was quantified in 100,000 cells based on a standard curve.

Cell viability assay: Human melanoma cell lines and isolated primary cultured human melanocytes were propagated and tested in early passage (Passages 7 to 9). The effects of NNT inhibitors (2,3BD, DCC, and Palmitoyl coenzyme A lithium salt) on cell viability were evaluated by the CellTiter-Glo Luminescent Cell Viability Assay (Promega, #G7570) and measurement of luminescence was performed on an EnVision 2104 Multilabel Reader (PerkinElmer). Human melanoma cell lines and primary melanocytes were plated on 96-well white plates (10,000 cells/well) and were treated with the NNT inhibitors at the indicated concentrations for 24 h.

Glutathione measurements: Cell lysates were prepared from equal numbers of cells after 24 h of DCC or 2,3BD treatment, following the manufacturer's protocols. Seventy-two h post siRNA treatment or overexpression of NNT and their corresponding controls, glutathione levels were determined using the GSH/GSSG-Glo assay (Promega, #V6611) and luminescence was measured using an EnVision 2104 Multilabel Reader (PerkinElmer).

Determination of NADPH/NADP ratio: Cell lysates were prepared from equal numbers of UACC257 human melanoma cells 72 h post siRNA treatment or overexpression of NNT and their corresponding controls. NADPH/NADP⁺ ratios were determined using the NADP/NADPH-Glo Assay (Promega, #G9082) following the manufacturer's protocol and luminescence was measured using an EnVision 2104 Multilabel Reader (PerkinElmer).

Luciferase reporter assay: To measure MITF transcriptional activity. UACC257 melanoma cell lines were infected with the dual-reporter system (GeneCopoeia. #HPRM39435-LvPM02), which expresses secreted Gaussia luciferase (GLuc) under the TRPM1 promoter and SEAP (secreted alkaline phosphatase) as an internal control for signal normalization. The cells were grown in complete RPMI medium containing 10% Fetal Plex. Medium was collected 24, 48, and 72 h post siRNA transfection. GLuc and SEAP activities were measured by Secrete-Pair Gaussia Luciferase Assay Kit (GeneCopoeia, #LF062) and QUANTI-Blue™ Solution (Invivogen, #rep-qbs), respectively, according to the manufacturers' instructions.

Histology and Immunofluorescence: For histology, paraffin sections were prepared and stained with hematoxylin and eosin (H&E) using the ihisto service (ihisto.io/). For visualization of melanin, paraffin sections were stained using a Fontana-Masson Stain kit (abcam, #ab150669). Briefly, the samples were incubated in warmed Ammoniacal silver solution for 30 min, followed by a Nuclear Fast Red stain.

For immunofluorescence, paraffin sections were deparaffinized by xylene and rehydrated gradually with ethanol to distilled water. Sections were submerged in 0.01 M citrate buffer and boiled for 10 min for retrieval of antigen. The sections were washed with TBST (0.1% Tween 20) and blocked with protein blocking solution (Agilent, #X090930-2) for 1 h at room temperature before application of primary antibody [1:100 diluted in Antibody Diluent (DAKO, #S3022)] and incubation overnight at 4° C. The following day, sections were washed with TBST three times and incubated with secondary antibody Alexa Fluor 647 goat anti-mouse IgG (G+L) (ThermoFisher Scientific, #A-21236), Alexa Fluor 594 F(ab)2 fragment of goat anti-rabbit IgG (G+L) (ThermoFisher Scientific, #A-11072), or Alexa Fluor 555 goat anti-rabbit IgG (ThermoFisher Scientific, #A-21428). After washing, the tissue sections were cover-slipped with mounting medium (SlowFade® Gold Antifade Reagent with DAPI, ThermoFisher Scientific, #S36939). MaxBlock Autofluorescence Reducing Reagent Kit (MaxVision Biosciences, #MB-L) was used to quench skin tissue autofluorescence according to the reagent instructions.

The following primary antibodies were used at the indicated dilutions (antibody sources are in the Key Resources Table): anti-CPDs monoclonal antibody (1:1,500), rabbit anti-gamma-H2AX (P-ser139) polyclonal antibody (1:5,000), rabbit anti-NNT (C-terminal) polyclonal antibody (1:100), rabbit anti-gamma-H2AX [p Ser139] polyclonal antibody (1:100).

Primary human melanocytes (50,000 cells/well) were cultured on chamber slides (ThermoFisher Scientific, #125657). Seventy-two hours post siRNA transfection, the cells were fixed with 4% paraformaldehyde (PFA) (ThermoFisher Scientific, #50980487) for 20 min at room temperature, followed by treatment with 0.1% Triton X-100 (Sigma) for 5 min and blocking with 10% goat serum (Sigma Aldrich, #G9023) containing 5% BSA in PBS for 60 min atroom temperature. Mouse anti-NNT monoclonal antibody [8B4BB10] was diluted with the blocking solution to a final concentration of 5 μg/ml and incubated with the cells overnight at 4° C. The following day, the slides were washed with TBST three times and incubated with donkey anti-mouse Alexa Fluor 488 secondary antibody (1:500). Sections were washed with TBST three times and mounted in mounting medium (VECTASHIELD® HardSet™ Antifade Mounting Medium with DAPI, Vector Laboratories, #H-1500). Images were captured using confocal microscopy (Zeiss Axio Observer Z1 Inverted Phase Contrast Fluorescence microscope).

Detection of cellular reactive oxygen species (ROS): The redox-sensitive fluorescent dye chloromethyl-2′, 7′-dichlorodihydrofluorescein diacetates (CM-H2DCFDA, ThermoFisher Scientific, #C6827) was used to measure intracellular ROS accumulation. UACC257 melanoma cells were cultured on a glass bottom dish and treated with the indicated siRNAs. Forty-eight h post siRNA treatment, 2 μM CM-H2DCFDA in PBS/5% FBS was added and the samples were incubated at 37° C. for 30 min to assess overall ROS production. Subsequently, the cells were incubated with 5 μM MitoSOX Red (ThermoFisher Scientific, #M36008) in PBS/5% FBS at 37° C. for 10 min, washed with HBSS, and analyzed by immunofluorescence imaging (Zeiss Axio Observer Z1 Inverted Phase Contrast Fluorescence microscope). The results were normalized to cell numbers, which were determined by nuclear staining with 1 drop per ml of NucBlue (ThermoFisher Scientific, #R37605) at 37° C. for 15 min.

Transmission electron microscopy: Cultured primary human melanocytes were grown in Medium 254 in 6-well transwell plates. Ninety-six h post siRNA or overexpression treatment, the cells were fixed with a modified Kamovsky's fixative (2% paraformaldehyde/2.5% glutaraldehyde in 0.1 M sodium cacodylate buffer, pH 7.4) for at least 2 h on a gentle rotator, followed by rinsing several times with 0.1 M cacodylate buffer. Then, the cells were treated with 1% osmium tetroxide/0.1 M cacodylate buffer for 1 h, rinsed thoroughly in 0.1 M cacodylate buffer, scraped, and the cell suspensions were transferred into 15 ml centrifuge tubes and centrifuged (3,000 rpm) for 15 min at 4° C.). Pelleted material was embedded in 2% agarose, dehydrated through an ethanol gradient (series of solutions from 30% to 100% ethanol), dehydrated briefly in 100% propylene oxide, then allowed to infiltrate overnight on a gentle rotator in a 1:1 mix of propylene oxide and Eponate resin (Ted Pella, Inc., kit with DMP30, #18010′). The following day, specimens were transferred into fresh 100% Eponate resin for 2-3 hours, then embedded in flat molds in 100% fresh Eponate resin, and embeddings were allowed to polymerize for 24-48 h at 60° C. Thin (70 nm) sections were cut using a Leica EM UC7 ultramicrotome, collected onto formvar-coated grids, stained with 2% uranyl acetate and Reynold's lead citrate, and examined in a JEOL JEM 1011 transmission electron microscope at 80 kV. Images were collected using an AMT digital imaging system with proprietary image capture software (Advanced Microscopy Techniques, Danvers, MA). Measurements of distances between melanosomes and mitochondria were quantified in FIJI (ImageJ) (Schindelin et al., 2012) by applying a customized macro to TEM micrographs. Melanosomes (N=˜50) were randomly selected for each condition within the whole image data set. Thirty Euclidean distances from the melanosome surface to the closest mitochondria surface were measured in nm. From these 30 single measurements the mean was calculated to give a final single mean value per melanosome-mitochondria event. A total of ˜50 events (N) were quantified per condition. Data were plotted and statistically analyzed using Prism 8 (Version 8.4.3). Melanosome-mitochondria distances closer than 20 nm were considered melanosome-mitochondria close appositions or contacts, consistent with (Daniele et al., 2014). Cell area (μm²), number of melanosome-mitochondria contacts, and number of mitochondria were quantified in FIJI (ImageJ) using polygon and multi-point selection tools. Melanosome identification and quantification were performed with images at 40,000× magnification or higher. Stages were estimated based on morphological features previously noted, namely multivesicular endosomes (Stage 1), unpigmented fibrils (Stage 1l), pigmented fibrils (stage Ill), and darkly pigmented filled melanosomes (Stage IV). All identifiable melanosomes in 4 cells per condition were quantified and classified, and the proportions of each stage were normalized to cell cytosolic area (determined by ImageJ).

Tyrosinase activity assay: UACC257 human melanoma cells were treated with human NNT siRNA or non-targeting siRNA control pool for 4 days. Cell lysates were prepared by adding 1% Trion X100 in PBS for 1 h at room temperature with shaking. Tyrosinase activity was measured as previously described (lozumi et al., 1993). Briefly, freshly made 25 mM L-DOPA in PBS was heated and added to the cell lysates in a 96-well plate. L-DOPA levels were determined by measuring the absorbance at 490 nm with shaking for 30 cycles, compared with mushroom tyrosinase (Sigma-Aldrich #T3824, 0 to 50 μg/μl in PBS), using an Envision 2104 Multilabel plate reader (PerkinElmer).

Human genetic association studies: For all cohorts, the GRCh37/hg19 human genome build was used. SNPs with minor allele frequency less than 1% were excluded from each cohort.

### A. The Rotterdam Study:

Population: The Rotterdam Study (RS) is a prospective population-based follow-up study of the determinants and prognosis of chronic diseases in middle age and elderly participants (aged 45 years and older) living in the Ommoord district (Rotterdam, the Netherlands) (Ikram et al., 2017). The RS consists of 4,694 people of predominantly North European ancestry. Phenotyping: As part of the dermatological investigation within the RS, participants from three cohorts (RSI, RSII and RSIII) were screened to assess their skin color. In brief, trained physicians scored the skin color of the participants using a scale from 1 to 6, with 1 for albino, 2 for white, 3 for white to olive color, 4 for light brown, 5 for brown, and 6 for dark brown to black. The reliability of the assessment has been validated before (Jacobs et al., 2015). Individuals with dark skin were excluded since they were likely to have a different genetic background than Europeans.

Genotyping and imputation: The RS-I and RS-II cohorts were genotyped with the Infinium II HumanHap550K Genotyping BeadChip version 3 (Illumina, San Diego, California USA) and the RS-III cohort was genotyped using the Illumina Human 610 Quad BeadChip. The RS-I, RS-II and RS-III cohorts were imputed separately using 1000 Genomes phase 3 (Genomes Project et al., 2012) as the reference dataset. Quality control on the single nucleotide polymorphisms (SNPs) has been described before (Hofman et al., 2015). SNPs were filtered out if they had a minor allele frequency of less than 1% or an imputation quality (R2) of less than 0.3. We used MACH software for the imputation with parameter defaults. Best-guess genotypes were called using the GCTA program (Yang et al., 2011) with parameter defaults. Statistical analysis: We used a multivariate linear regression model to test for associations between SNPs within the NNT region and skin color in the RS using an additive model (Purcell et al., 2007). The model was adjusted for age, sex and four principal components (variables derived from principal component analysis that were added to correct for possible population stratification and hidden relatedness between participants). The PLINK program was used for conducting associations.

### B. The CANDELA Cohort:

A GWAS study of skin color in the CANDELA cohort has been published (Adhikari et al., 2019) and summary statistics are available at gwascentral.org/study/HGVST3308. Details of the cohort and analyses are in the published study, so only the cohort population and phenotyping are summarized here.

Population: 6,357 Latin American individuals were recruited in Brazil, Chile, Colombia, Mexico and Peru. Participants were mostly young, with an average age of 24.

Phenotyping: A quantitative measure of constitutive skin pigmentation (the Melanin Index, MI) was obtained using a DermaSpectrometer DSMEII reflectometer (Cortex Technology, Hadsund, Denmark). The MI was recorded from both inner arms and the mean of the two readings was used in the analyses.

Statistical analysis: P-values for SNPs in the NNT region were obtained from the published CANDELA summary statistics.

### C. The East & South African Cohort

The summary statistics were obtained from a previous study of pigmentation evolution in Africans (Crawford et al., 2017). Details of the cohort and analyses are in the published study, so only the cohort population and phenotyping are summarized here.

Population: A total of 1,570 ethnically and genetically diverse Africans living in Ethiopia, Tanzania, and Botswana were sampled in this cohort.

Phenotyping: A DSM II ColorMeter was used to quantify reflectance from the inner underarm.

Reflectance values were converted to a standard melanin index score.

Statistical analysis: P-values for SNPs in the NNT region were obtained from the published summary statistics.

### D. The UK Blobank Cohort:

There have been many published studies on pigmentation phenotypes in the UK Biobank (Jiang et al., 2019) and the summary statistics are publicly available at cnsgenomics.com/software/gcta/#DataResource. Details of the cohort and analyses are in the published study, so only the cohort population and phenotyping are summarized here.

Population: The UK Biobank includes more than 500,000 individuals from across the UK, with predominantly White British ancestry.

Phenotyping: Self-reported categorical questions were used to record data on skin color and ease of skin tanning.

For skin color, 6 categories were used: very fair, fair, light olive, dark olive, brown, and black (biobank.ctsu.ox.ac.uk/crystal/field.cgi?id=1717). 450,264 responses were available.

For ease of skin tanning (biobank.ctsu.ox.ac.uk/crystal/field.cgi?id=1727), participants were asked “What would happen to your skin if it was repeatedly exposed to bright sunlight without any protection?” Four categories were used: very, moderately, mildly, and never tanned. 446,744 responses were available.

For sun protection use (biobank.ctsu.ox.ac.uk/crystaVfield.cgi?id=2267), participants were asked “Do you wear sun protection (e.g., sunscreen lotion, hat) when you spend time outdoors in the summer?” Four categories were used: never/rarely, sometimes, most of the time, and always. 452.925 responses were available.

Statistical analysis: P-values for SNPs in the NNT region were obtained from the published UK Biobank summary statistics.

Meta-analysis of the cohorts: Considering the huge variation in sample size among the 4 cohorts. Fisher's method (Won et al., 2009) of combining p-values from independent studies was used, in which p-values for one marker across different cohorts were combined to provide an aggregate p-value for the meta-analysis.

Multiple testing adjustment: Since we tested 332 independent associations, we corrected the significance threshold for multiple testing. We used the false discovery rate (FDR) method of controlling the multiple testing error rate, following the Benjamini-Hochberg procedure (Benjamini and Cohen, 2017). Applying the FDR procedure on the set of p-values to achieve an overall false positive level of 5%, the adjusted significance threshold was p=1.01E-3. As there is substantial LD (linkage disequilibrium) between the SNPs, a Bonferroni correction would have been overly conservative.

GWAS conditional on known pigmentation variants: MC1R is a major determinant of pigmentation, with known genetic variants associated with lighter skin color, red hair, and freckles in European populations (Quillen et al., 2019). Among the two European cohorts used in this study, individual-level data were only available for the Rotterdam Study, so the conditional GWAS analysis was conducted only in this cohort. We retrieved the dose allele of major MC1R variants data from the Rotterdam studies and used them as covariates in the earlier used multiple linear regression model, in addition to the previously mentioned covariates. The association P-value of the NNT variant is thus conditioned on the known pigmentation variants in this analysis. These conditioned P-values were then compared to the original (unconditioned) P-values with a Wilcoxon rank-sum test to assess whether they have been significantly altered due to the conditioning on the known pigmentation variants. Jacobs et al. 2015 examined three functional variants in MC1R for their relationship with pigmentation in the Rotterdam Study: rs1805007, rs1805008, rs1805009 (Jacobs et al., 2015). Therefore, the first conditional analysis was performed using these three MC1R variants. Subsequently, an additional set of well-established genetic variants in other pigmentation genes (Adhikari et al., 2019) were also used for conditioning: rs28777 (SLC45A2), rs12203592 (IRF4), rs1042602 (TYR), rs1800404 (OCA2), rs12913832 (HERC2), rs1426654 (SLC24A5), and rs885479 (MC1R).

Correlation between trait effect sizes and eQTL expression data: eQTL expression data corresponding to expression levels of the NNT transcript were downloaded from the GTEx database. For each genetic variant in the NNT region, we obtained the normalized effect size (NES) and P-value for the derived (non-reference) allele in each of the two skin tissues: “Skin—Not Sun Exposed (Suprapubic)” and “Skin—Sun Exposed (Lower leg)”. Correlation values were calculated between the regression coefficients for the derived (non-reference) alleles of each variant from the UK Biobank for each of the three traits and the NES values corresponding to the same alleles (to ensure consistency of effect direction) in each of the two skin tissues.

### Quantification and Statistical Analysis

ImageJ v1.8.0 (imagej.nih.gov/ij/) was used to quantify the immunoblots. FIJI software enabling pixel-based color quantification was used for Zebrafish analysis.

Statistical analyses were performed using GraphPad Prism 8. In general. for comparisons of two groups, significance was determined by two-tailed, unpaired Student's t tests, correcting for multiple t tests with the same two groups using the Holm-Šidák method. One-way and two-way ANOVA tests were used for comparisons of more than two groups involving effects of one or two factors, respectively, using the recommended post-tests for selected pairwise comparisons. The specific statistical tests used for experiments are described in the figure legends. P values less than 0.05 were considered statistically significant. Levels of significance are indicated by *p<0.05, *p<0.01, **p<0.001, ***p<0.0001; ns, not significant.

### Example 1. NNT Enables Regulation of Pigmentation Via Changing Intracellular Redox Levels

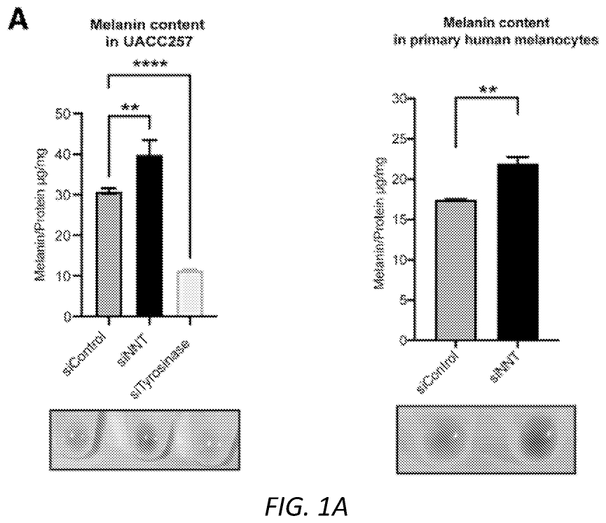

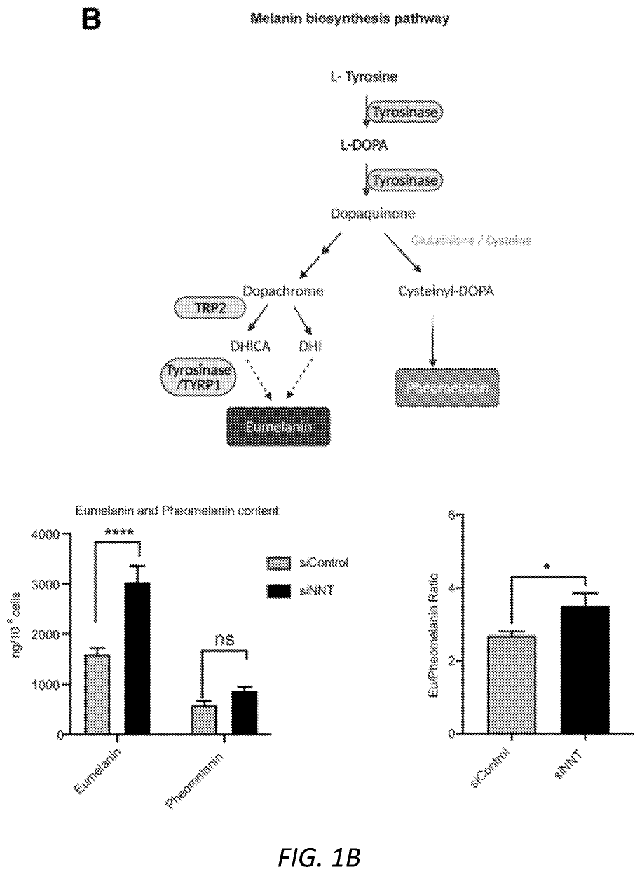

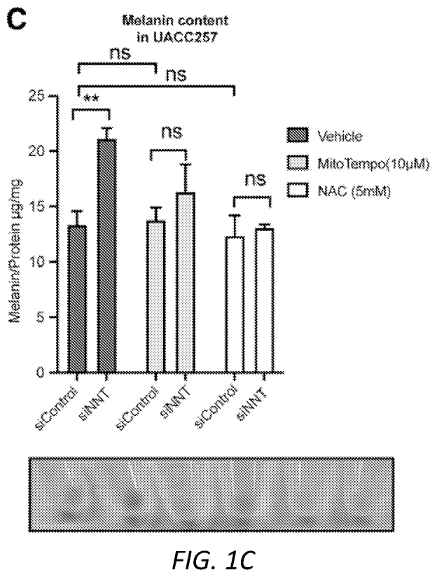

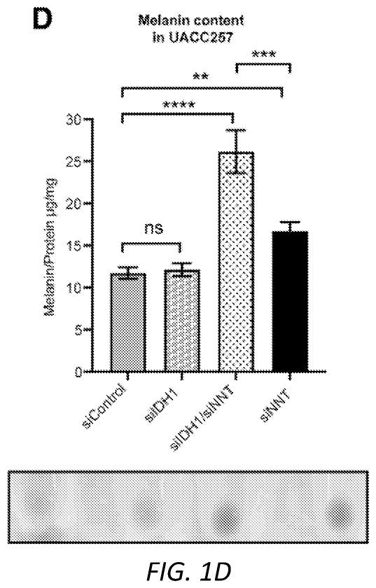

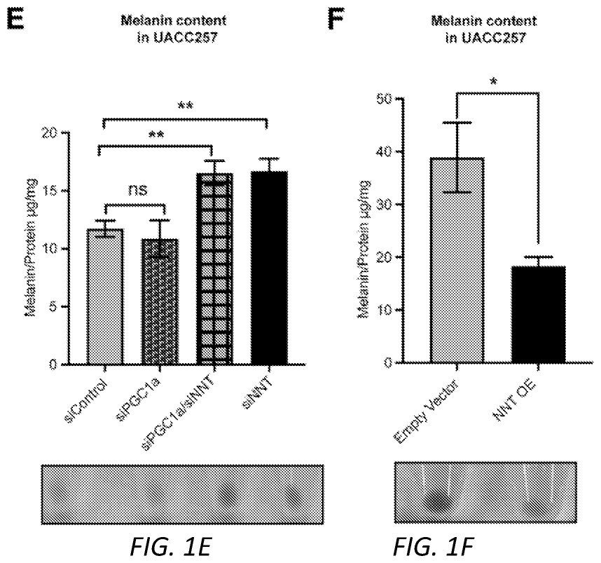

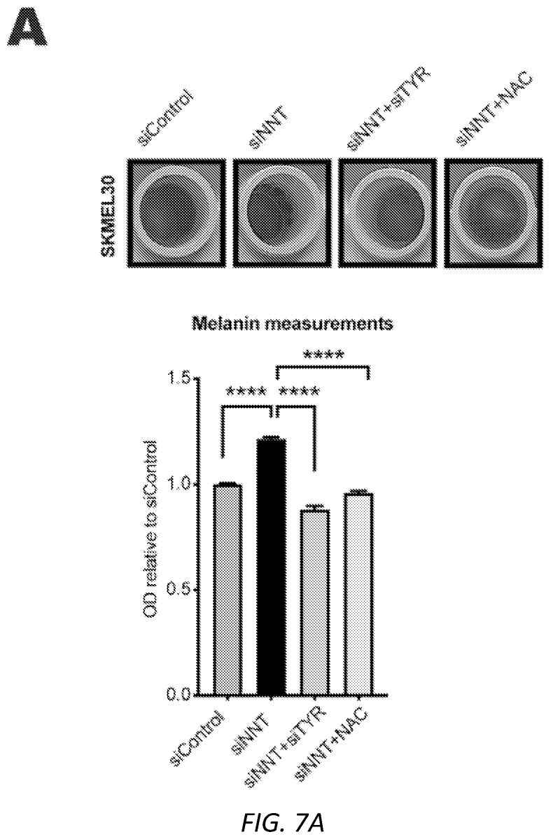

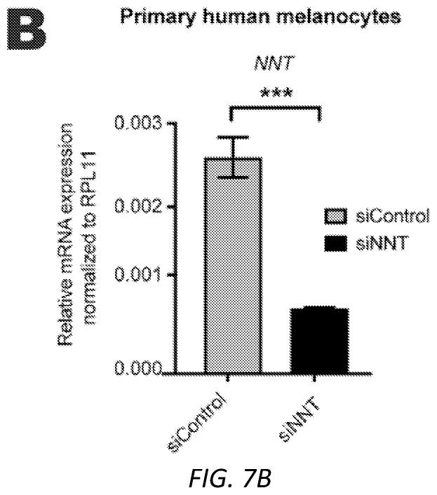

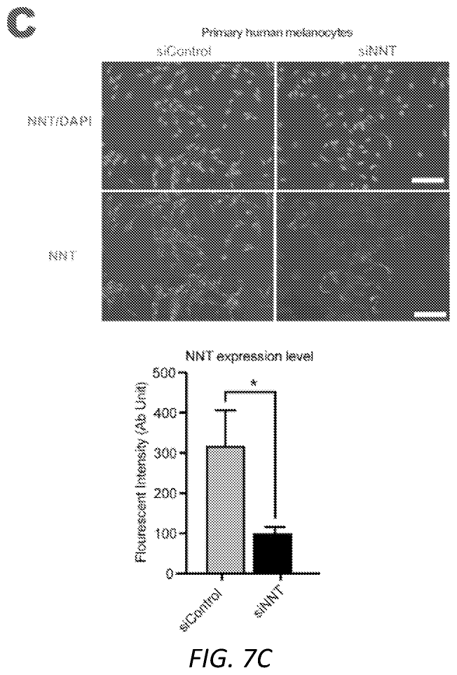

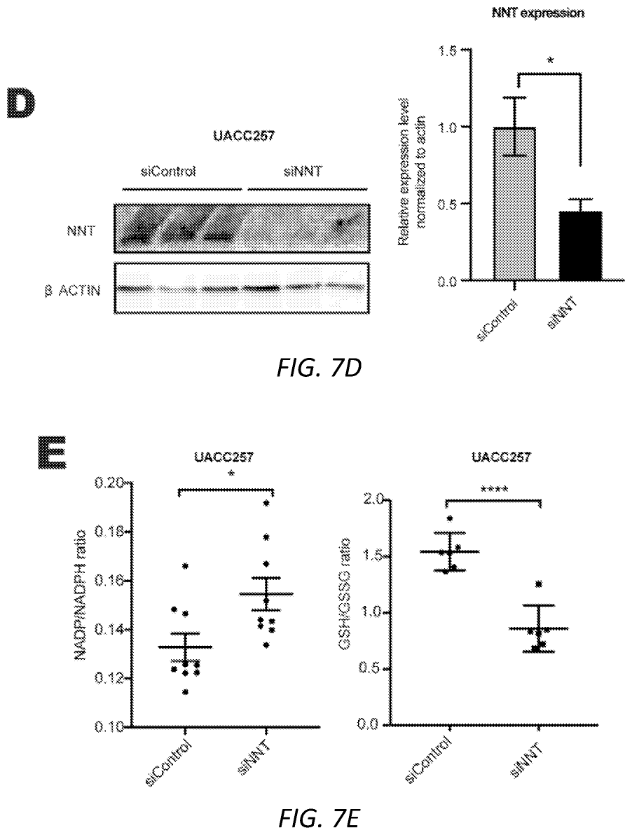

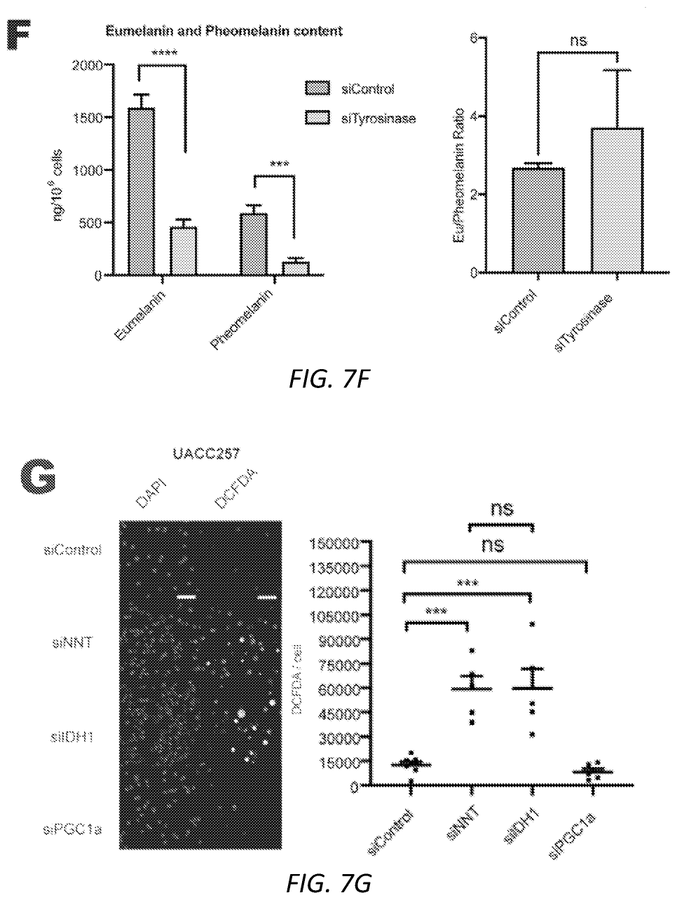

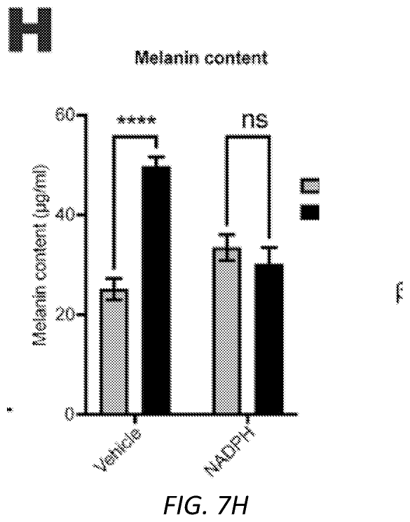

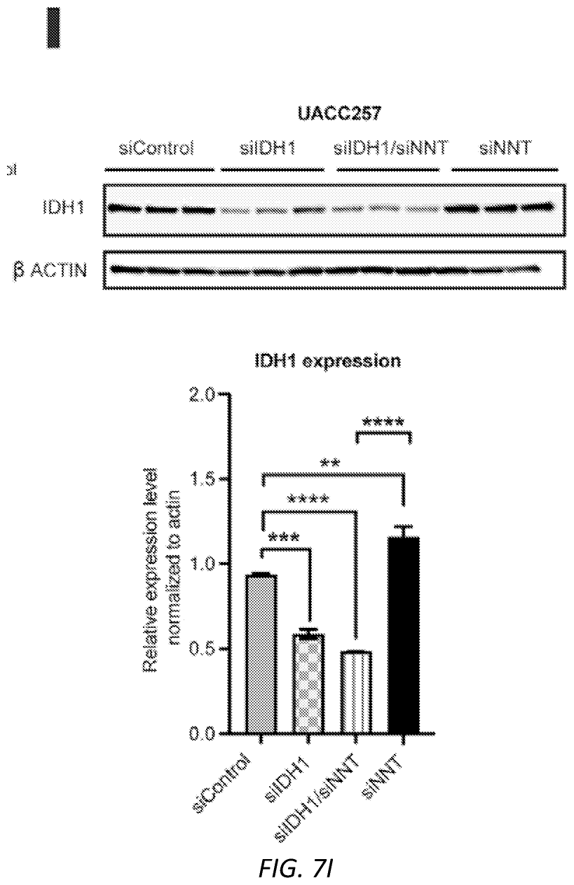

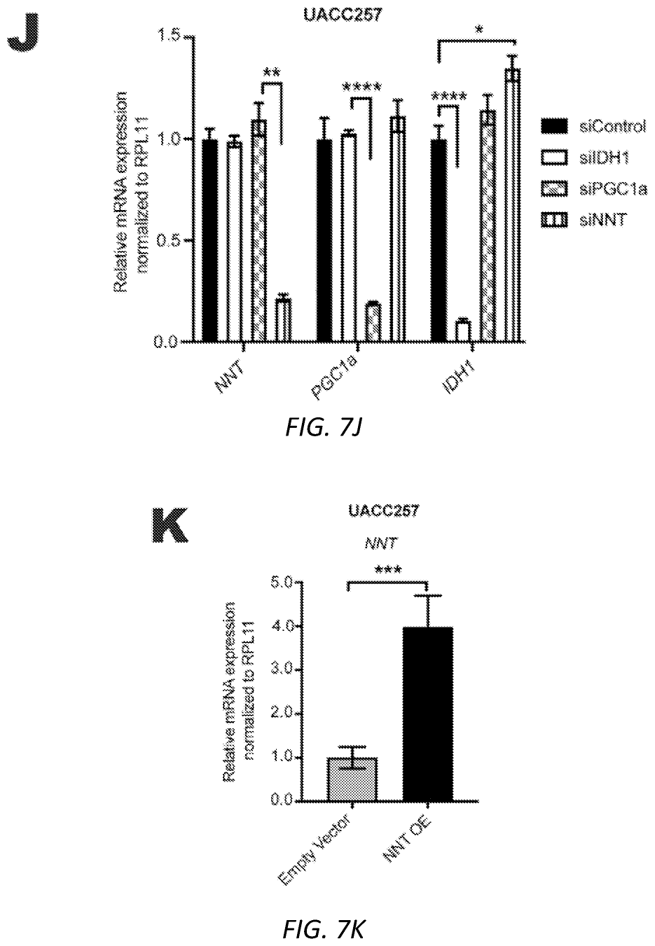

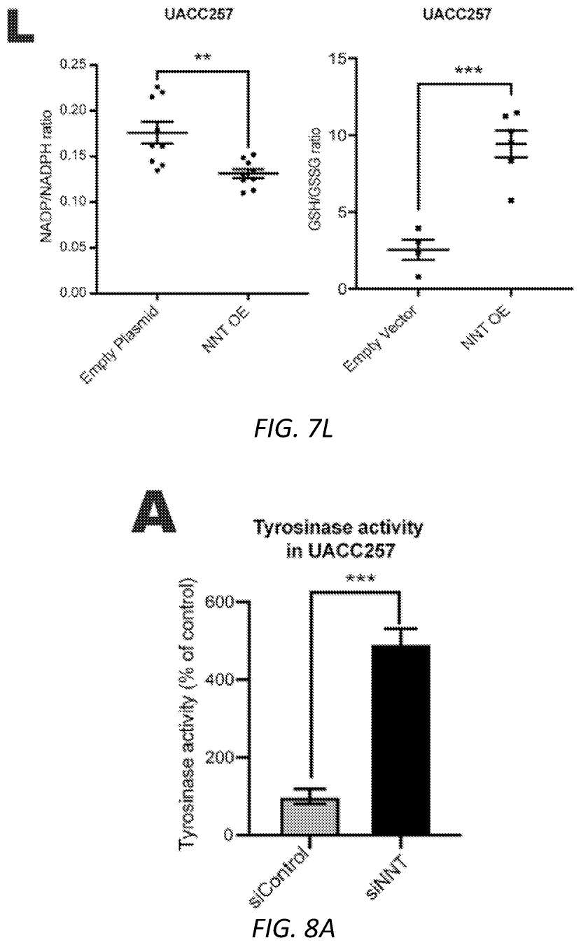

NNT was depleted using a pool of siRNAs (siNNT) in human melanoma cell lines UACC257 and SK-MEL-30, and in primary human melanocytes. In all three cell models knockdown of NNT led to a significant increase in melanin content (FIGS. 1A, 7A-D). The increase of pigmentation following siNNT was blocked by simultaneous knockdown of tyrosinase demonstrating the dependence of siNNT-mediated pigmentation on tyrosinase (FIG. 7A).

NNT has been described to increase GSH in Nnt wild type versus Nnt mutant C57BL/6J mice (Ronchi et al., 2013), as well as in human myocardium (Sheeran et al., 2010). In line with this, silencing NNT caused a decrease of the GSH/GSSG ratio in UACC257 human melanoma cells (FIG. 7E). Cysteine or reduced glutathione is a required component for pheomelanin synthesis (Ito and Ifpcs, 2003; Jara et al., 1988) (Schema, FIG. 1B), suggesting that NNT may modulate pigmentation via its role in regenerating GSH and thereby affecting the pheomelanin to eumelanin ratio. To investigate this possibility, high-performance liquid chromatography (HPLC) was utilized and demonstrated significantly increased absolute levels of eumelanin, but not pheomelanin, upon NNT knockdown (FIG. 1B, Left graph). The eumelanin to pheomelanin ratio also showed a significant increase, (FIG. 1B. Right graph). Tyrosinase silencing was used as a positive control showing efficient and quick depigmentation five days after transfection (FIG. 1A), resulting in decreased levels of both eumelanin and pheomelanin, and as suspected, no significant change in the eumelanin to pheomelanin ratio (FIG. 7F). This data suggests that NNT modulates melanin synthesis towards a eumelanin phenotype.

Due to NNT's essential role as an antioxidant enzyme against ROS by controlling the NADPH conversion, we hypothesized that the increase in pigmentation following silencing of NNT is driven by an oxidative stress-dependent mechanism. As expected, knockdown of NNT caused a significant increase in the NADP/NADPH ratio (FIG. 7E) and induced cytosolic ROS (FIG. 7G) in UACC257 cells. Adding thiol antioxidant N-acetylcysteine (NAC), mitochondria-targeted antioxidant MitoTEMPO, or NADPH to siNNT, inhibited the siNNT-mediated increase in pigmentation (FIGS. 1C, 7A and 7H), demonstrating the dependence of siNNT-mediated pigmentation on oxidative stress.

To understand how cytosolic and mitochondrial oxidative stress levels are connected, isocitrate dehydrogenase 1 (IDH1), a source of cytosolic NADPH (Zhao and McAlister-Henn, 1996) was depleted in UACC257 cells (FIGS. 1D, 7I and 7J). Interestingly, while siNNT alone increased pigmentation, silDH1 alone had no significant effect on pigmentation (FIG. 1D). However, the double knockdown of NNT and IDH1 increased the intracellular melanin content further, exceeding the siNNT-induction of pigmentation (FIG. 1D). To exclude the possibility that siIDH1 or siIDH1-induced oxidative stress may increase NNT levels, NNT mRNA levels were measured (FIG. 7I-J), which showed no changes. To understand if cytosolic ROS may be the driver of the observed pigmentation change, cytosolic oxidative stress was measured upon silencing of siNNT and silDH1 (FIG. 7G), showing similar effects of the different siRNAs, emphasizing the crucial role of NNT in human pigmentation.

In order to clarify the role of mitochondrial oxidative stress, we investigated the participation of peroxisome proliferator-activated receptor gamma coactivator 1-alpha (PGC1α). As shown previously, intramitochondrial concentrations of ROS were significantly increased in PGC1α-depleted melanoma cells, associated with decreased levels of reduced glutathione (GSH), cystathionine, and 5-adenosylhomocysteine (Vazquez et al., 2013). However, no change of pigmentation was detected in PGC1α-depleted human UACC257 melanoma cells (FIGS. 1E and 7J), thus emphasizing the specific role of NNT and especially NNT-induced cytosolic oxidative stress for the pigmentation response. Finally, overexpression of NNT in UACC257 cells (FIG. 7K) increased GSH/GSSG ratios and deceased NADP/NADPH ratios (FIG. 7L). As opposed to the increase in pigmentation observed with silencing of NNT, overexpression of NNT induced a significant decrease in pigmentation (FIG. 1F), confirming the relationship between NNT and pigmentation in both directions.

Taken together our data suggest that NNT affects pigmentation via a redox-dependent mechanism.

### Example 2. NNT Depletion Enhances Pigmentation Independently of the Classic cAMP-MITF-Pigmentation Pathway

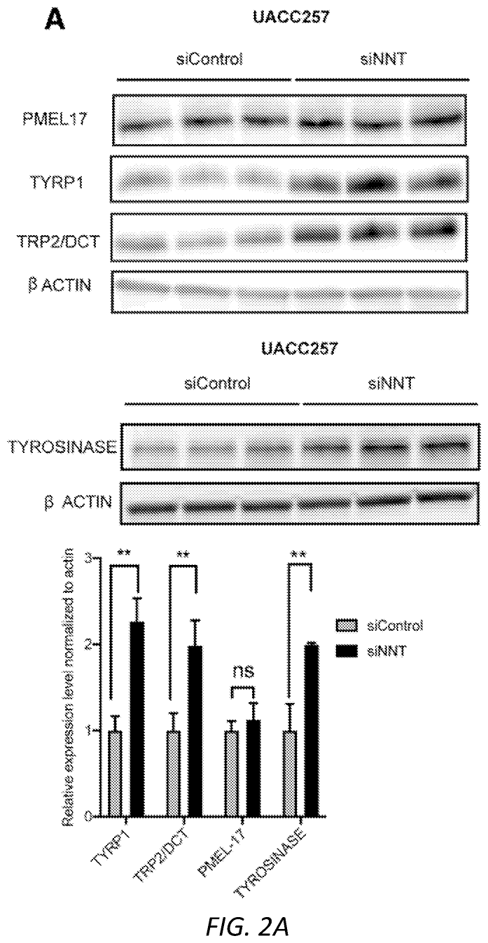

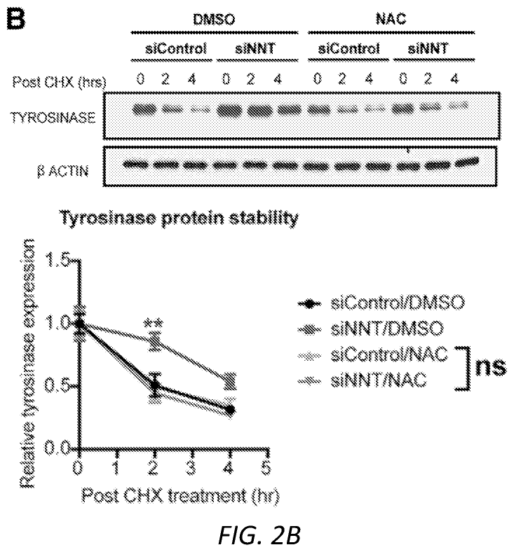

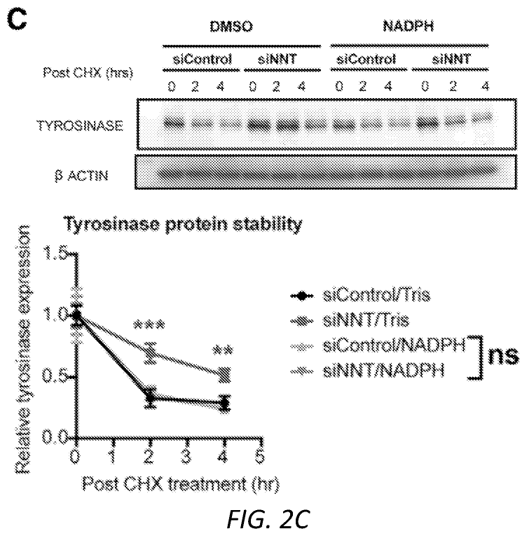

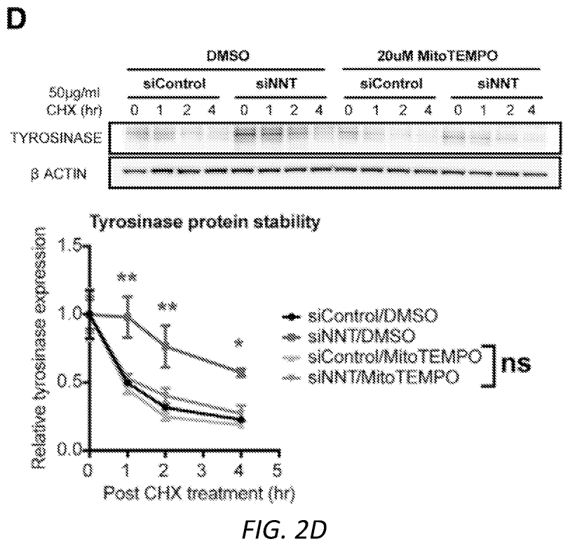

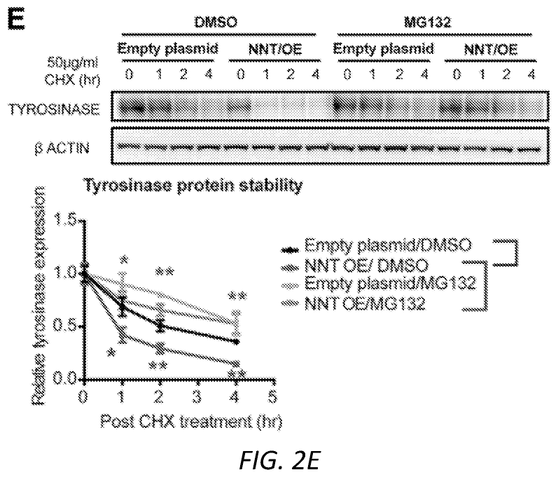

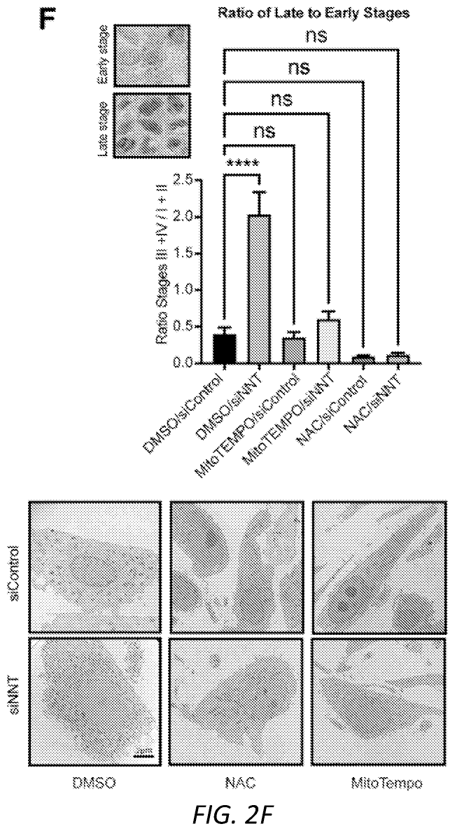

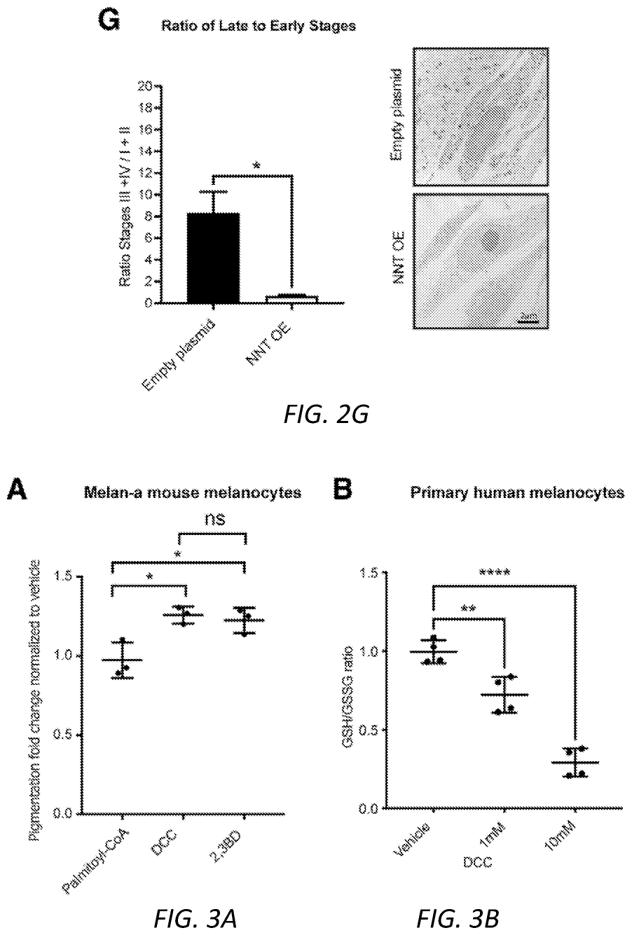

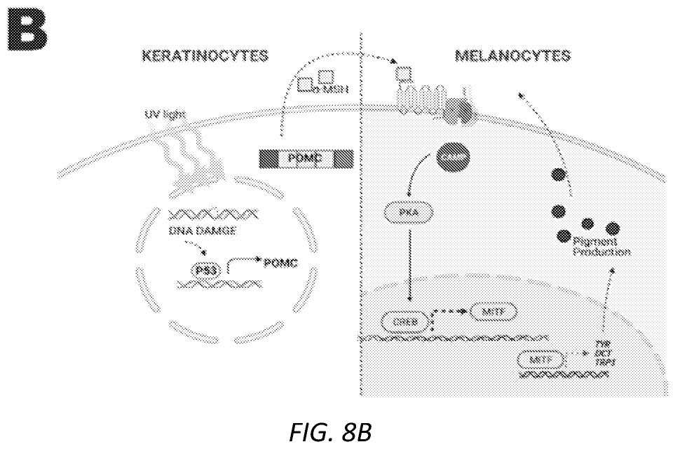

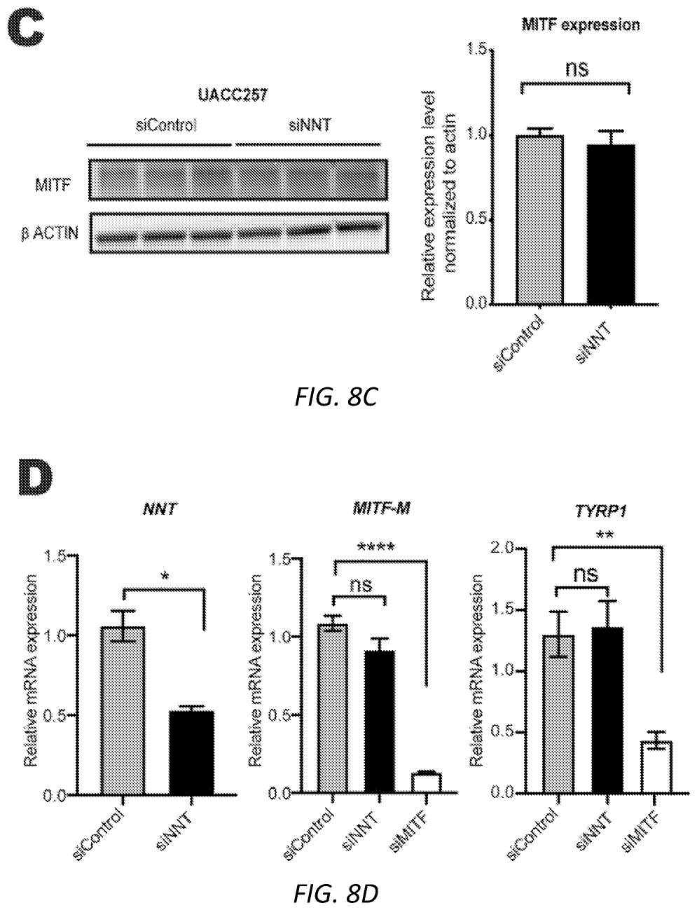

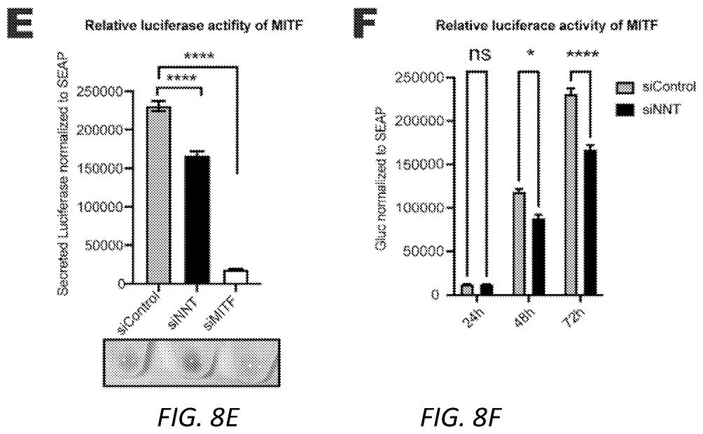

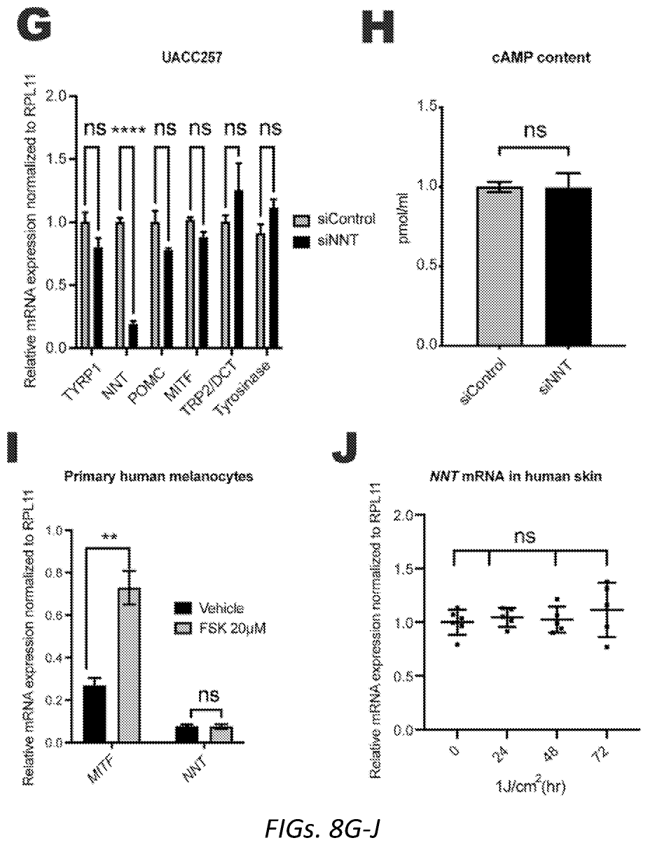

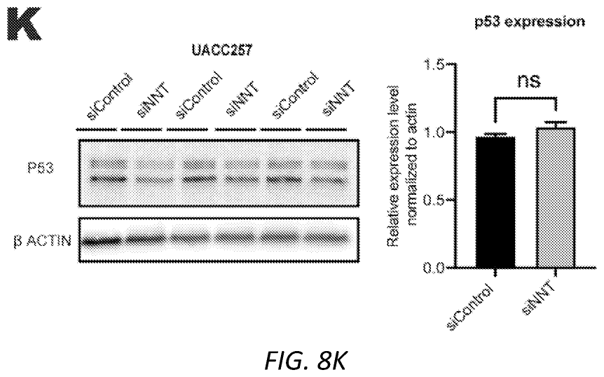

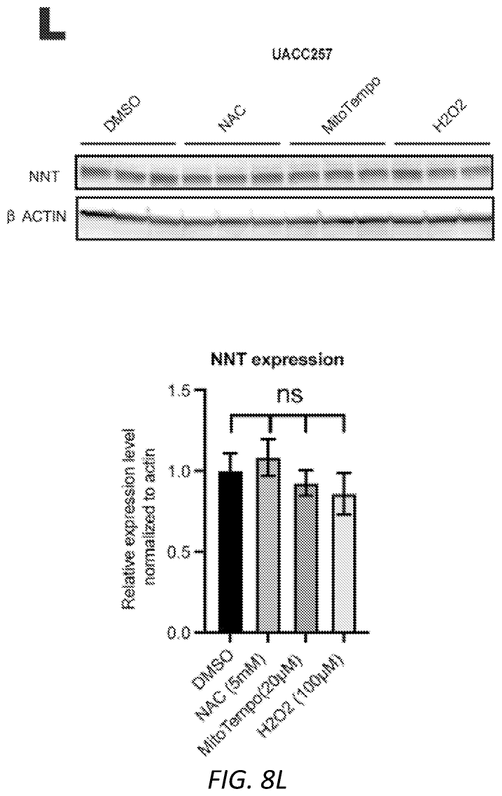

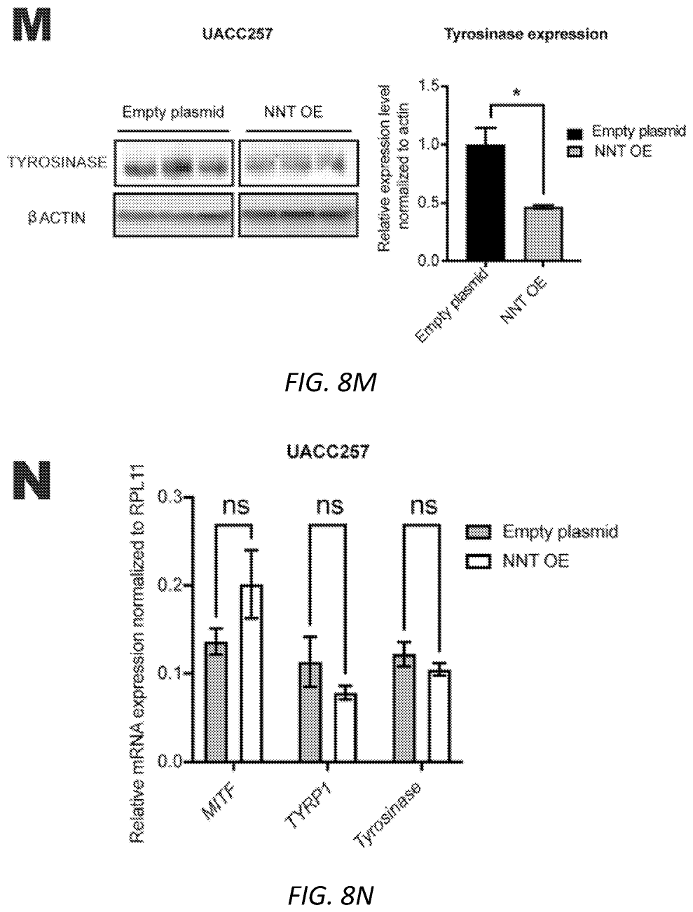

In order to elucidate the mechanism underlying hyperpigmentation after NNT knockdown, we investigated its effects on key melanin biosynthesis factors in UACC257 cells (FIG. 2A). NNT knockdown revealed a significant increase in the levels of the melanin biosynthesis enzymes, tyrosinase, TYRP1 and TRP2/DCT (FIG. 2A). In addition, tyrosinase activity was increased upon silencing of siNNT (FIG. 8A). Since MITF is the main regulator of these enzymes and the master regulator of melanogenesis (FIGS. 8B-G), we measured MITF protein levels and its transcriptional activity. Upon silencing of NNT, neither MITF protein levels, nor mRNA levels were significantly changed (FIGS. 8C-D). Furthermore, MITF promoter activity was modestly decreased following siNNT (FIGS. 8E-F), while no significant change in the mRNA level of TYRP1, TRP2/DCT or tyrosinase was observed (FIG. 8G). This suggests that NNT can impact tyrosinase. TRP2/DCT and TYRP1 protein levels without affecting their mRNA levels. As cAMP is a crucial messenger in UV-induced skin pigmentation (“classic cAMP-MITF-pigmentation pathway”) (FIG. 8B), baseline cAMP levels in siControl-vs. siNNT-transfected UACC257 cells were assayed and found to be unaffected by siNNT (FIG. 8H). Treatment of primary human melanocytes with forskolin, an activator of adenylate cyclase, which increases cAMP levels, did not affect NNT expression levels (FIG. 8I), nor did UVB irradiation of human skin (FIG. 8J). In addition, no increase in POMC (FIG. 8G) or p53 (FIG. 8K) was observed in UACC257 cells upon siNNT treatment. Further, modulating the general redox system by adding NAC, MitoTEMPO or H₂O₂ did not impact NNT protein levels (FIG. 8L).

Finally, overexpression of NNT in UACC257 showed a significant decrease of tyrosinase protein levels (FIG. 8M) but not its mRNA levels (FIG. (8N).

Together, these data suggest the existence of an NNT-dependent pigmentation mechanism, independent of the previously established cAMP-MITF-dependent pigmentation pathway.

### Example 3. NNT Promotes Ubiquitin-Proteasome-Dependent Tyrosinase Degradation and Modulates Melanosome Maturation

Since altering NNT was found to impact the protein levels of tyrosinase and related key melanogenic enzymes (FIG. 2A) without impacting their mRNA levels (FIG. 8G), we hypothesized that NNT can affect the stability of certain melanosomal proteins. The impact of NNT-mediated redox changes on tyrosinase protein stability was investigated by knockdown of NNT mRNA in the presence or absence of an antioxidant, followed by inhibition of protein synthesis with cycloheximide (CHX) and measurements of the rate of decay of tyrosinase protein. Silencing of NNT increased tyrosinase protein stability significantly, and this effect was prevented by antioxidant treatment with either NAC, NADPH or Mito-Tempo (FIGS. 2B-D).

The mechanism of tyrosinase degradation is not fully understood, although it has been shown that tyrosinase is degraded via the ubiquitin-proteasome system (Bellei et al., 2010). Addition of carbobenzoxy-L-leucyl-L-leucyl-L-leucinal (MG132), a cell-permeable, reversible proteasome inhibitor prevented an NNT overexpression-induced decrease in Tyrosinase protein stability in UACC257 cells (FIG. 2E), suggesting that NNT induces changes in melanin levels is through proteasome-mediated degradation of Tyrosinase protein.

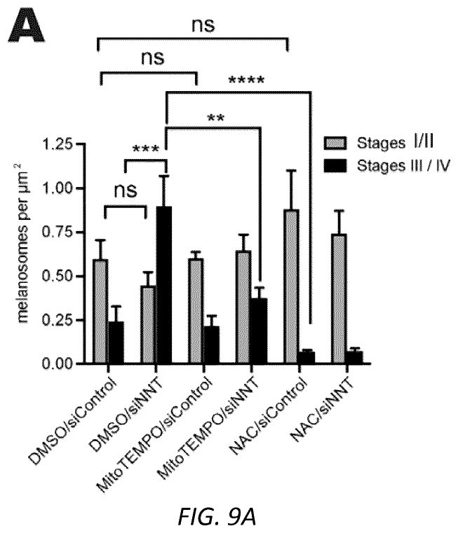

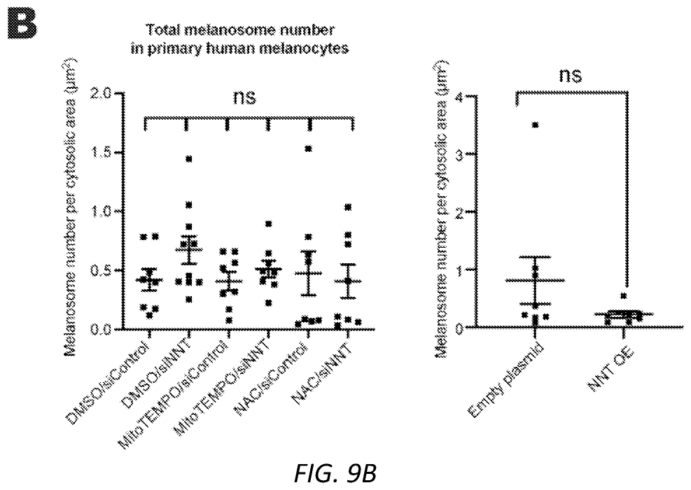

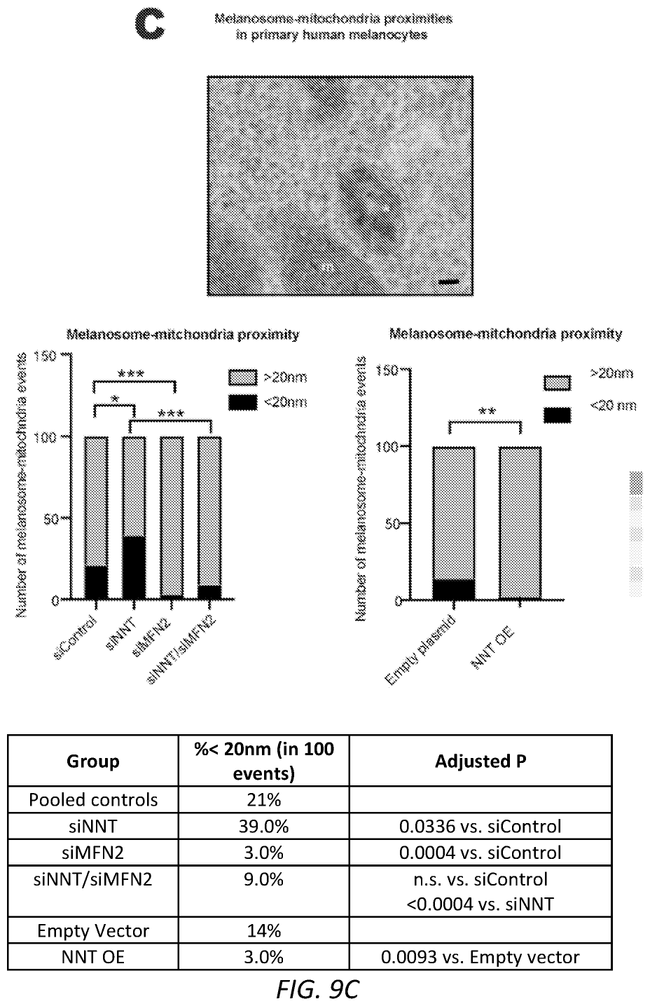

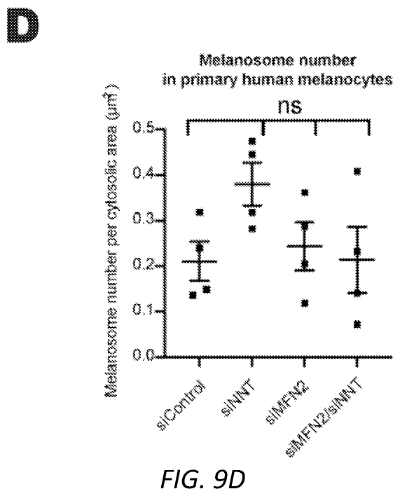

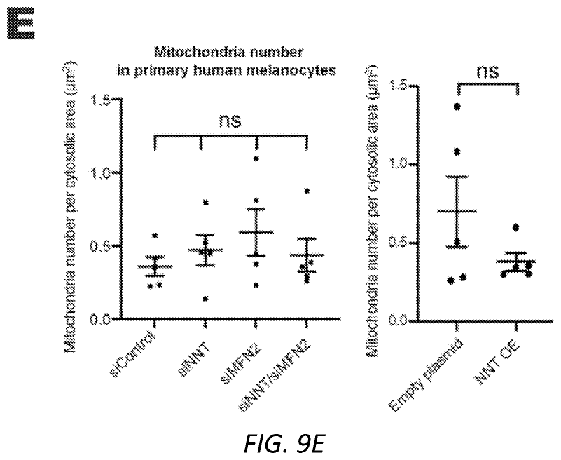

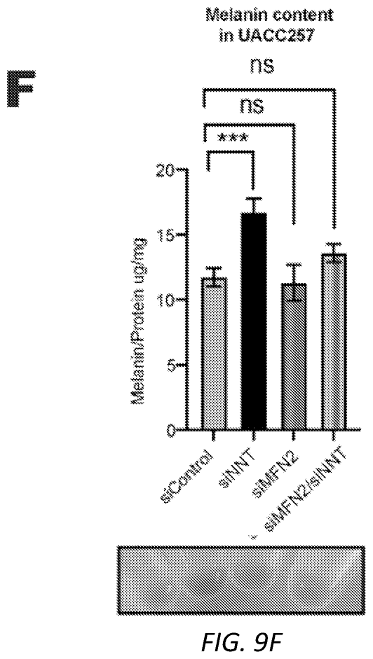

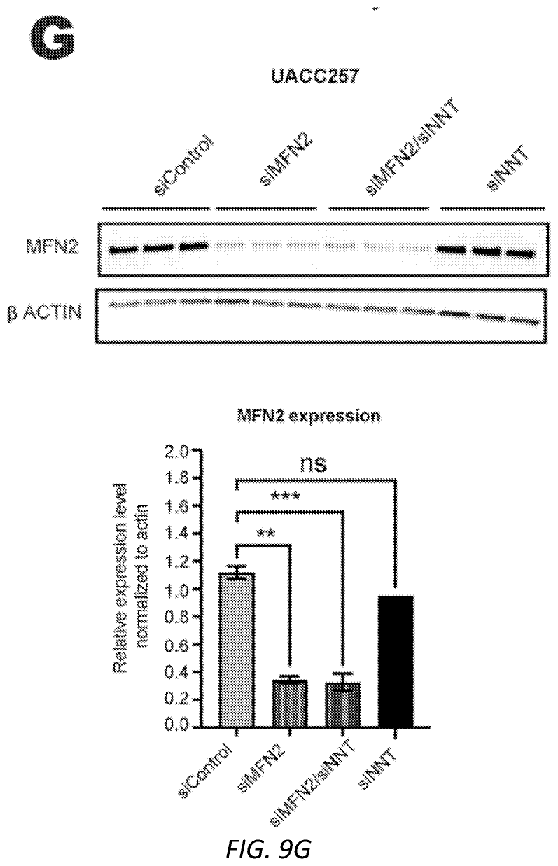

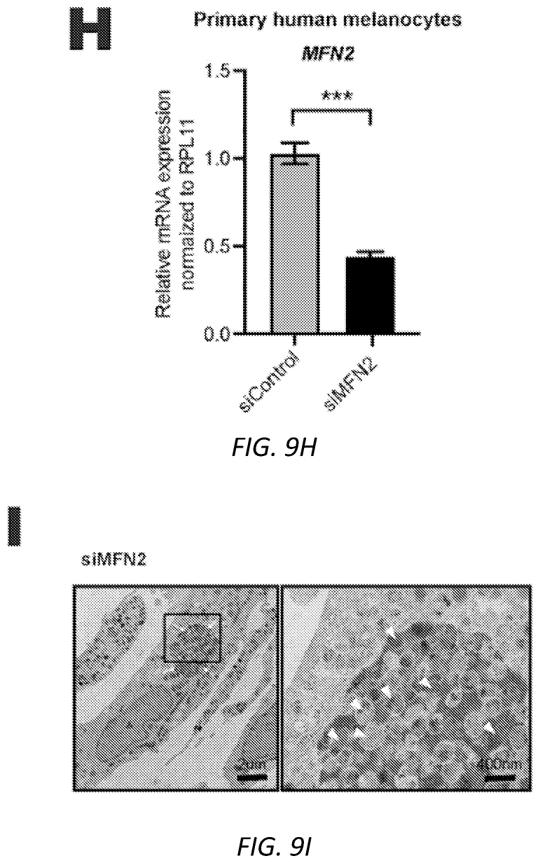

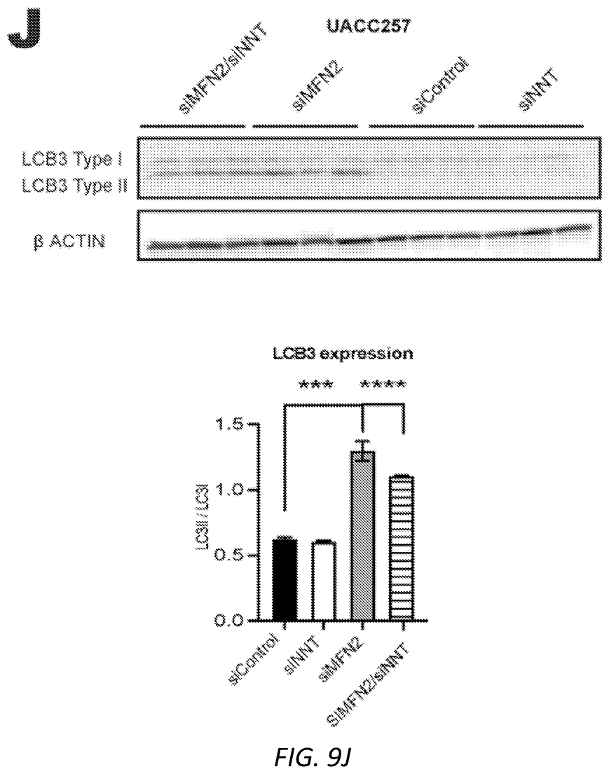

Due to siNNT-induced increases in melanogenesis enzymes, NNT's role in NADPH and GSH generation and its location in the inner mitochondrial membrane, we hypothesized that NNT function might be connected to the maturation of melanosomes. The effects of modulating NNT expression on the ultrastructure of melanosomes was assessed by electron microscopy in primary human melanocytes. Knockdown of NNT resulted in a striking increase in late-stage/pigmentated melanosomes (stages III and IV) (FIGS. 2F and 9A), while overexpression of NNT resulted in a switch towards early-stage/unpigmented melanosomes (stages I and II) (FIG. 2G), establishing a role for NNT in regulating melanosome maturation. In line with the pigmentation data (FIG. 1C), cotreatment with either NAC or MitoTEMPO prevented the siNNT-induced phenotype (FIGS. 2F and 9A). The absolute number of melanosomes per cytosolic area was not affected by NNT knockdown or overexpression (FIG. 9B), which is in line with the observation that the pre-melanosome protein Pmel17, a marker for early melanosome development, did not change upon depletion of NNT (FIG. 2A). Together, our data suggest that inhibition of NNT drives pigmentation via stabilizing tyrosinase and possibly other tyrosinase-related proteins (TYRP1 and TRP2/DCT) associated with increased melanosome maturation.

Previously, it has been shown that mitochondria are connected with melanosomes via physical contacts, requiring Mitofusin-2 (MFN2) (Daniele et al., 2014). The connection between these two organelles may enable localized interorganellar exchange (Daniele et al., 2014); (Wu and Hammer, 2014). To understand if siNNT-induced pigmentation may rely on an equivalent mechanism, we performed simultaneous knockdown of NNT and MFN2 in UACC257 cells (FIG. 9G) and in human primary melanocytes (FIG. 9H). Consistent with previous findings (Daniele et al., 2014) evaluation of mitochondria-melanosome proximities by electron microscopy confirmed that knockdown of MFN2, resulted in a strong decrease in close appositions (<20 nm) compared to control (FIG. 9C). By contrast, silencing of NNT alone lead to a relative increase of organelle contiguities, possibly related to the stimulation of melanogenesis (FIG. 9C), and double knockdown prevented this increase (FIG. 9C) while melanosome and mitochondria numbers remained unchanged (FIGS. 9D-E). Similar to the melanosome-mitochondria proximity, silencing of NNT in UACC257 human melanoma cells significantly increased the intracellular melanin content which was reversed by the simultaneous knockdown of NNT and MFN2 (FIG. 9F). Finally, overexpression of NNT resulted in decrease in close appositions (<20 nm) compared to control (FIG. 9C), while no change was observed in both melanosome and mitochondria numbers (FIGS. 9B and 9E).

While these findings suggest that MFN2 and melanosome-mitochondria proximity may contribute to NNT regulation of pigmentation changes, the role of MFN2 in melanogenesis is complex. In addition to interorganellar connections, MFN2 regulates many functions in cells, including mitochondrial fusion, ATP production, and autophagy, which may impact pigmentation (Filadi et al., 2018). In particular, MFN2 deficiency has been associated with impaired autophagic degradation and the accumulation of autophagosomes (Zhao et al., 2012); (Sebastian et al., 2016). Consistent with those findings, knockdown of MFN2 in human primary melanocytes and UACC257 cells resulted in the presence of large autophagosome-like structures containing numerous and partly intact melanosomes (FIG. 9I), as well as increased LCB3 Type II (9J), which can be associated with either enhanced autophagosome synthesis or reduced autophagosome degradation (Barth et al., 2010). Since defects of autophagosome formation and/or turnover interfere with melanosome biogenesis and are associated with pigmentary defects (Ho and Ganesan, 2011), we conclude that MFN2 can regulate pigmentation via distinct—incompletely understood—pathways.

### Example 4. Topical Nnt Inhibitors Increase Pigmentation

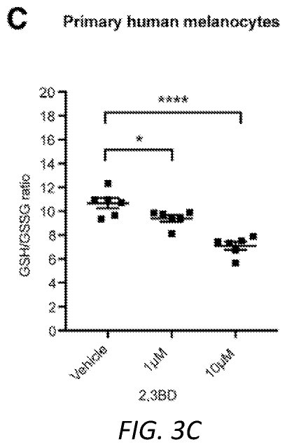

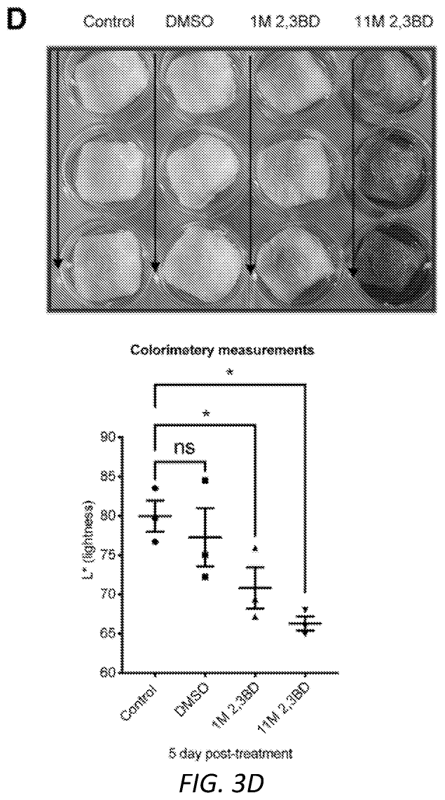

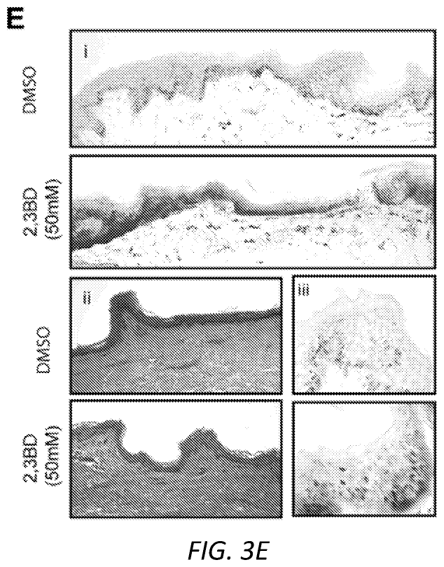

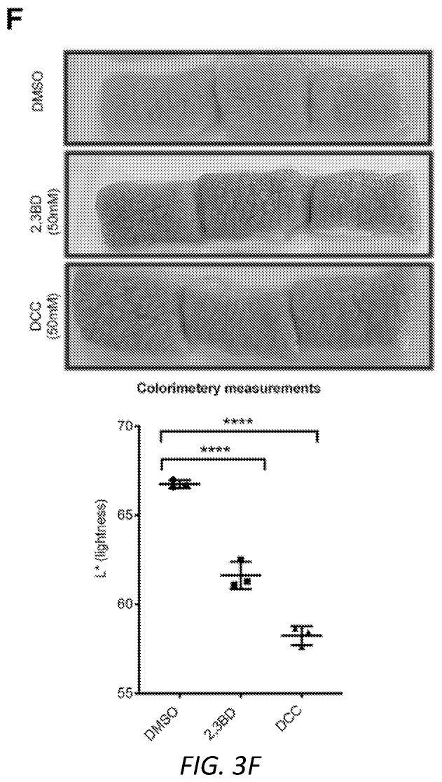

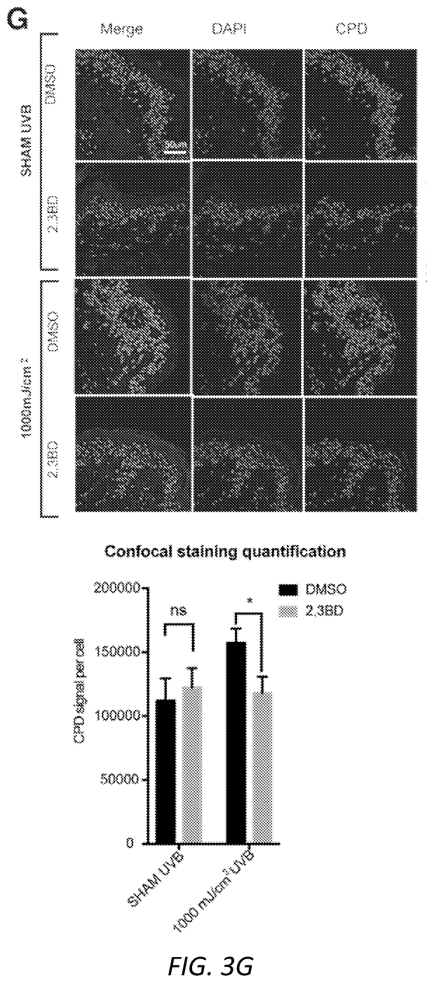

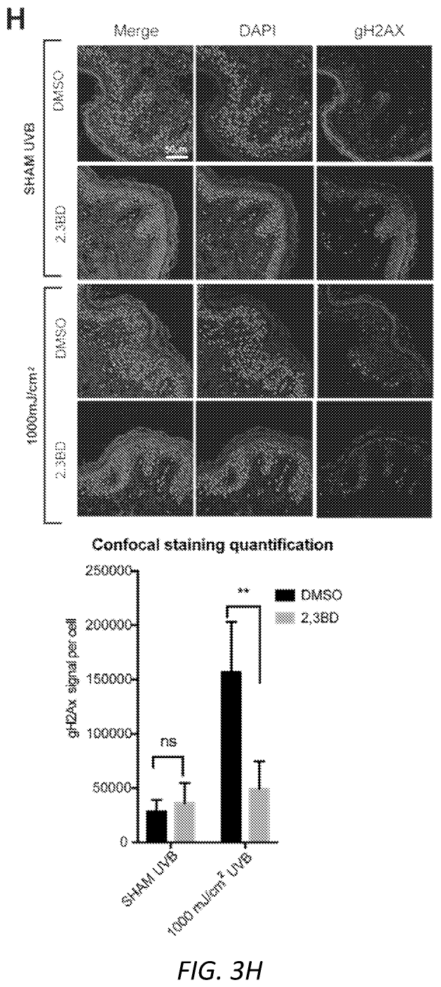

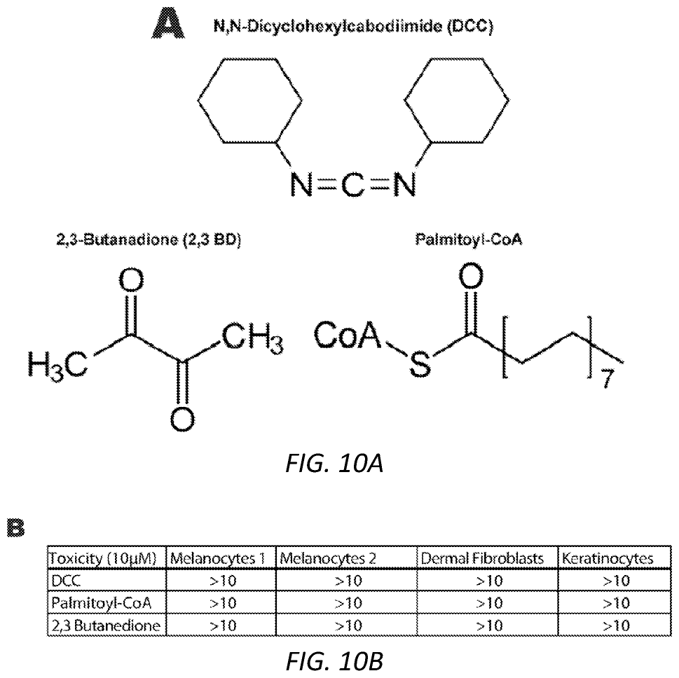

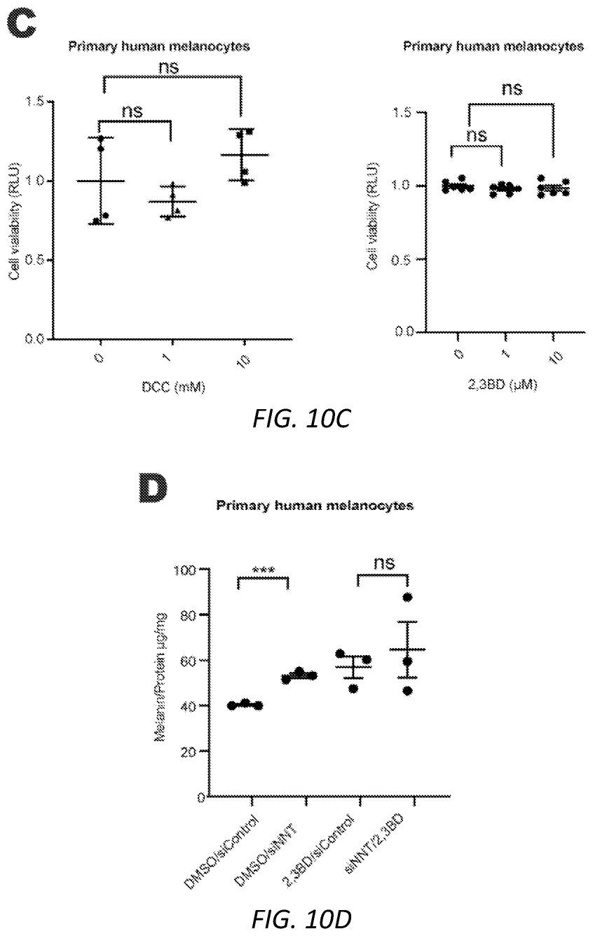

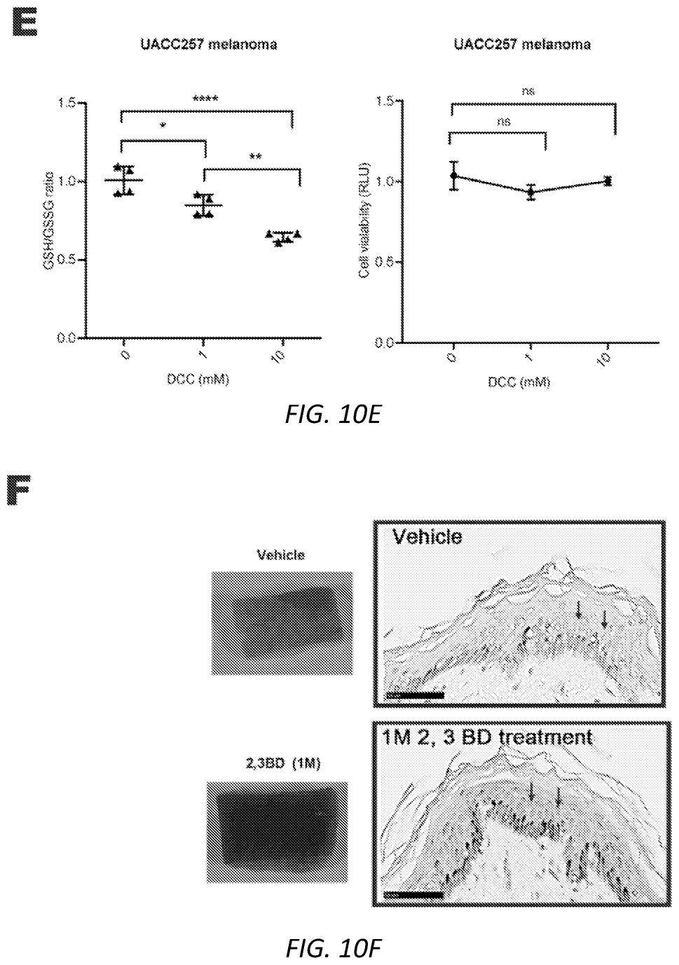

Currently, only a limited number of topical drugs are capable of altering pigmentation in human skin (Rendon and Gaviria, 2005). No topical skin darkeners are available for clinical use. Systemic administration of peptides such as α-MSH analogs (e.g., Melanotan) has been used to successfully increase skin pigmentation (Ugwu et al., 1997). Three NNT inhibitors (N,N′-Dicyclohexylcarbodiimide [DCC], 2,3-Butanedione [2,3BD], Palmitoyl-CoA) have been described previously (FIG. 10A) (Rydstrom, 1972). DCC is commonly used as a peptide-coupling reagent and 2,3BD is used as a flavoring agent (Rigler and Longo, 2010). Both are low molecular weight compounds (DCC: 206.33 g/mol; 2,3BD: 86.09 g/mol) potentially capable of penetrating human epidermis. Palmitoyl-CoA, like 2,3BD, is a natural product, but has a high molecular weight (1005.94 g/mol), making skin penetration challenging. The effects of all three compounds on pigmentation of intermediately pigmented murine Melan-A cells (FIG. 3A) were assessed. Both 2,3BD and DCC significantly increased the melanin content in intermediately pigmented murine Melan-A cells (FIG. 3A) and in human primary melanocyte (FIG. 10D). In vitro toxicity was assessed in primary human melanocytes, dermal fibroblasts and keratinocytes (FIG. 10B) showing no significant toxicity in doses up to 10∞M respectively, 100∞M for 2,3BD in primary melanocytes (FIG. 10C). To verify the effects of the small molecular weight compounds on NNT function, the GSH/GSSG ratio, an indirect endpoint of NNT enzyme activity, was measured, revealing decreased GSH/GSSG ratios induced by DCC and 2,3 BD in primary melanocytes (FIGS. 3B and 3C) and by DCC in UACC257 melanoma cells (FIG. 10E), without significant toxicity (FIGS. 10C and 10E). Treatment of primary human melanocytes with either siNNT or 2,3BD significantly increased the intracellular melanin content, however simultaneous treatment with siNNT and 2,3 BD did not further increase the melanin (FIG. 10D), suggesting that enhancement of pigmentation by 2,3 BD may be mediated by inhibition of NNT.

Next, we tested the compounds on human skin explants from different skin types. As suggested above, palmitoyl-CoA did not penetrate the epidermis and had no effect on pigmentation (data not shown). In abdominal skin from individuals of fair skin phototype 1-2, 2,3BD yielded a strong induction of pigmentation at relatively high doses (FIG. 3D). Histology with Fontana-Masson staining showed increased melanin in the 2,3BD treated skin (FIGS. 3Ei and 10F) and no obvious cell damage or inflammation by H&E staining (FIG. 3Eii), although the volatility of 2,3BD produces a strong butter-like aroma, potentially limiting its future clinical use. Importantly, keratinocytic supranuclear caps (FIG. 3Eiii and FIG. 10F) were present, suggesting the formation of functional melanosome/melanin transfer to keratinocytes, which allows cells to protect their nuclei from UV radiation. Daily application of 50 mM 2,3BD or DCC on skin from intermediately pigmented skin type 3-4 individuals yielded significantly increased pigmentation after 5 days (FIG. 3F). Due to the activity of DCC as a coupling agent and its corresponding unclear toxicity risks, only 2,3BD was used in subsequent experiments.

### Example 5. 2,3BD-Induced Skin Pigmentation can Prevent UVB-Induced DNA Damage

UV radiation interacting with DNA can directly produce cyclobutane pyrimidine dimers (CPD) and 6-4 photoproducts, whereas ROS-mediated DNA modifications produce alternative nucleotide adducts including 8,5-cyclo-2-deoxyadenosine, 8,5-cyclo-2-deoxyguanosine, and 8-oxo-deoxyguanine (Jaruga and Dizdaroglu, 2008; Wang, 2008).

While superficial epidermal cells containing modified proteins, lipids and DNA are continuously shed through corneocyte desquamation, durable basal cells require active DNA repair machinery for their maintenance. Melanomas have been found to contain high frequencies of somatic mutations with characteristic UV-induced signatures of C to T and G to A transitions (Berger et al., 2012). Protecting human skin from these intermediates is a major goal of skin cancer prevention strategies. As shown in previous studies, increased pigmentation can help to protect against CPD formation (D'Orazio et al., 2006; Mujahid et al., 2017). We tested if 2,3BD-induced pigmentation can protect skin from UVB-induced CPD formation. After inducing a visible increase in pigmentation of human skin by application of 50 mM 2,3BD to skin type 2-3 for 5 days (FIG. 3G), UVB was applied and CPD formation was detected by immunofluorescence staining and normalized to the total number of cells. It was observed that 2,3BD treatment protected against formation of UVB-induced CPD (FIG. 3G). We then measured γ-H2AX, a marker of DNA double-stranded breaks, in order to investigate potential 2,3BD-mediated toxicity as well as whether 2,3BD-mediated skin pigmentation could protect from UVB-induced γ-H2AX induction (FIG. 3H). 2,3BD was observed to be non-toxic and the pigmentation it produced could protect human skin from UVB-induced γ-H2AX induction.

### Example 6. NNT Regulates Pigmentation in Mice, Zebrafish and Human Pigmentation Disorders

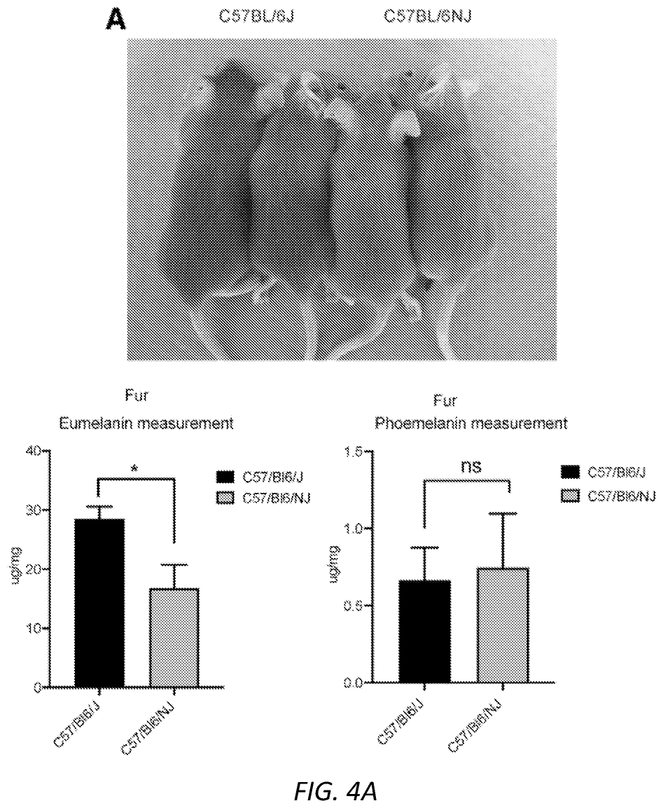

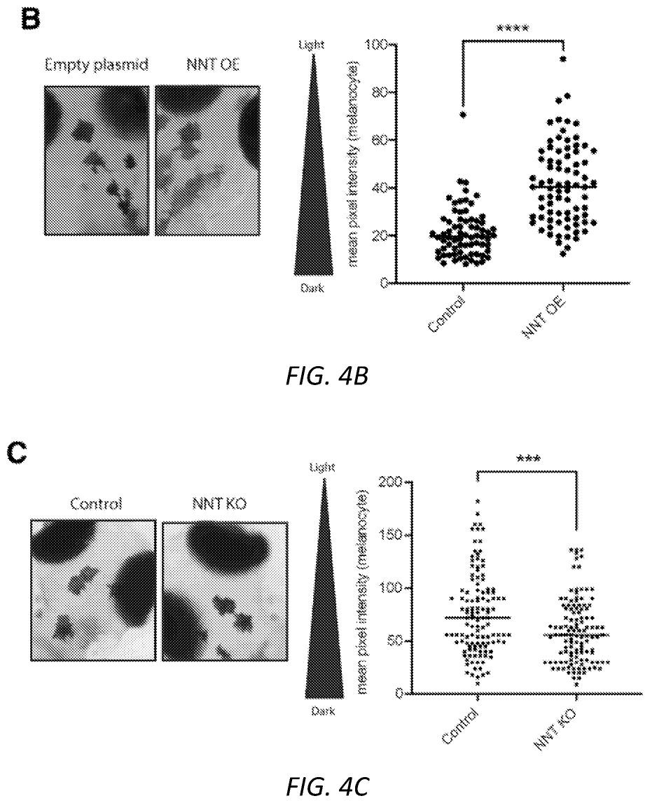

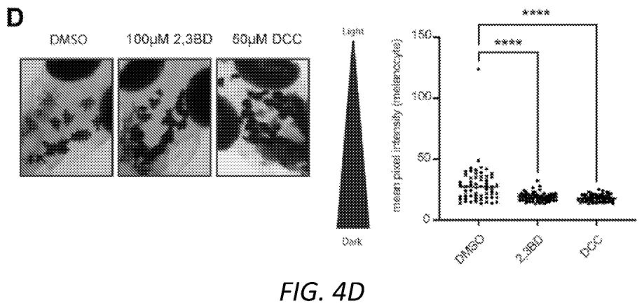

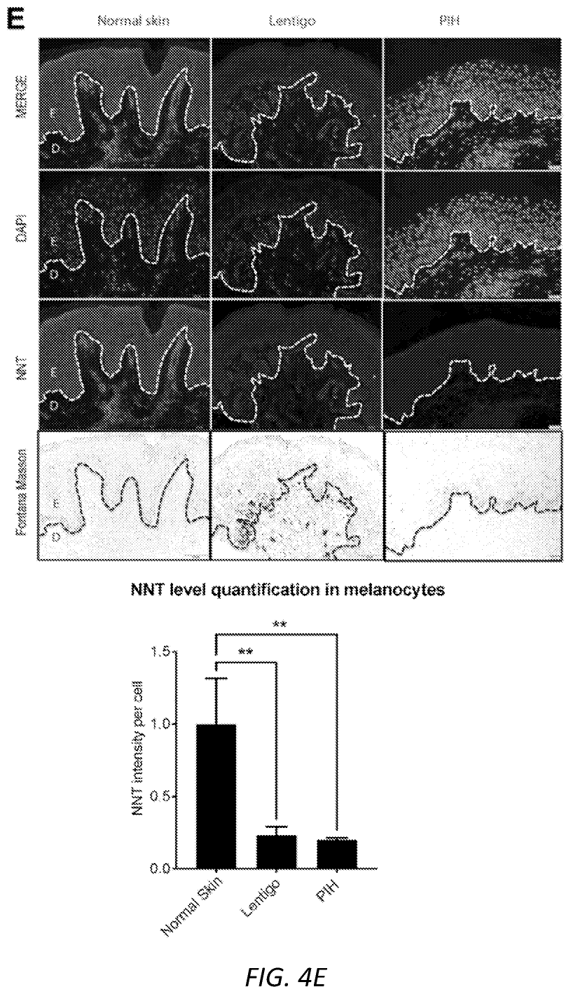

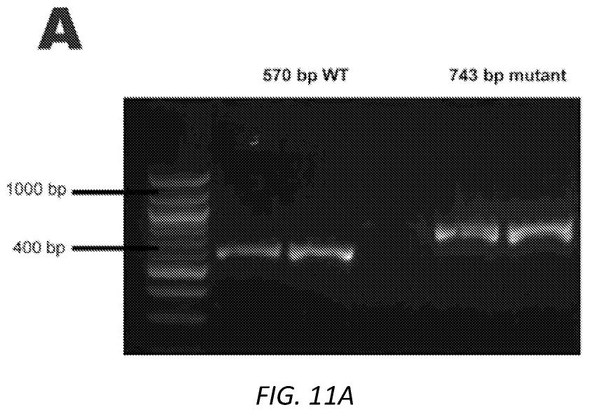

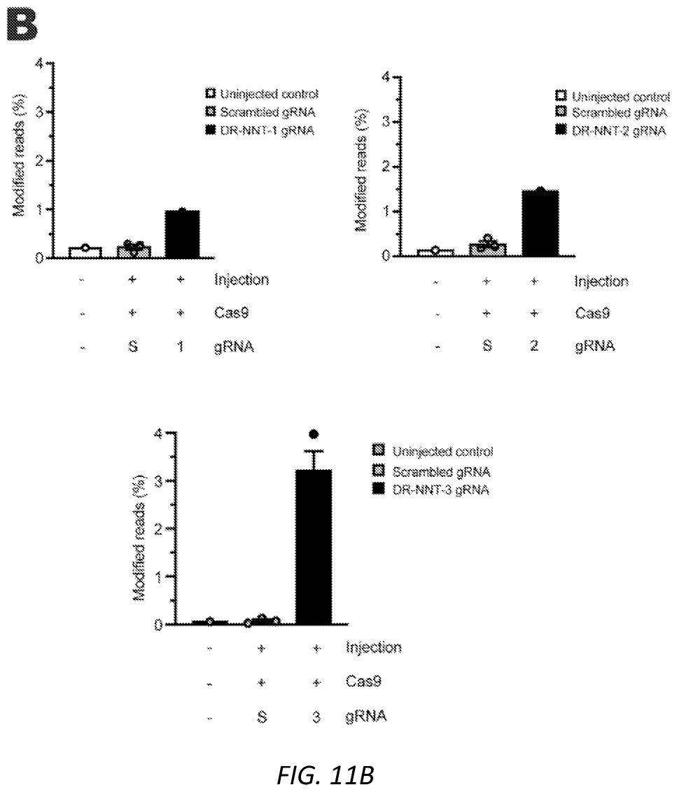

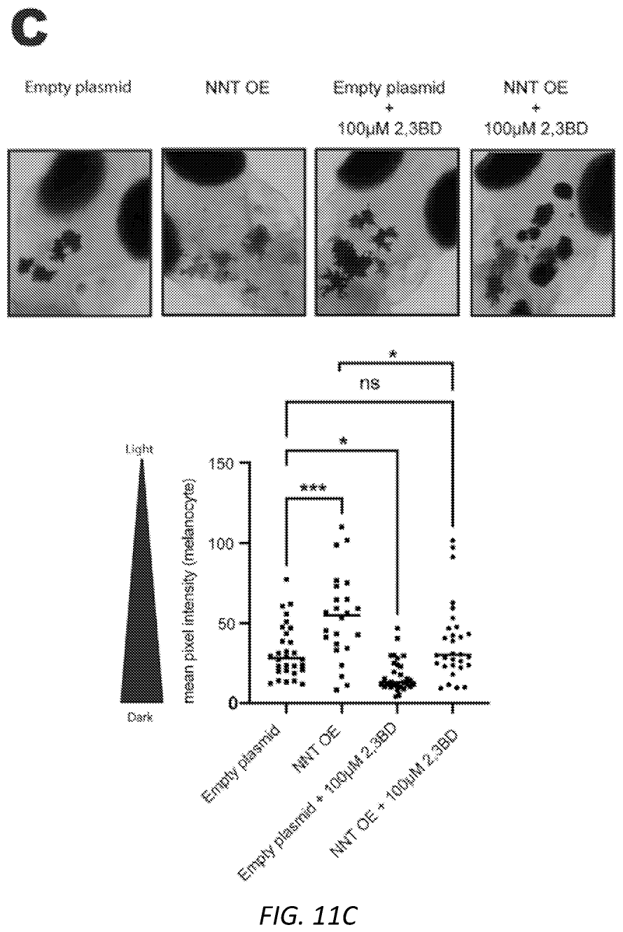

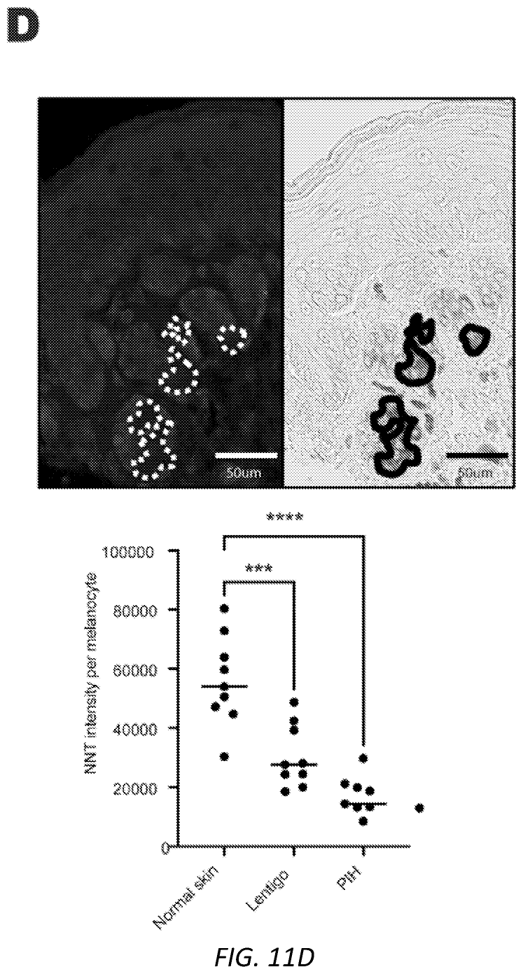

C57BL/6J and C57BL/6NJ mice are substrains of the C57BL/6 mouse with known genetic differences. While C57BU6NJ mice are homozygous for the Nnt wild type allele, C57BL6J mice are homozygous for the NntC57BL/6J mutation. This mutant allele is missing a stretch of 17,814 bp between exons 6 and 12, resulting in a lack of mature protein in these mutants (Toye et al., 2005) (Huang et al., 2006). In our experiments, C57BU6J mice that are homozygous for the Nnt mutation (FIG. 11A) showed increased fur pigmentation compared with C57BL6/NJ control (wild type Nnt) mice (FIG. 4A, Left panel). Quantification of pheomelanin and eumelanin levels in mouse hair by HPLC shows higher eumelanin, but not pheomelanin, in C57BU6J mice compared with C57BU6NJ mice (FIG. 4A).

Next, a zebrafish (Danio rero) model that overexpresses NNT selectively in melanocytes was engineered. Similar to humans and mice, zebrafish melanocytes originate from the neural crest, and the pathways leading to melanocyte differentiation and pigment production are conserved. Many human pigmentation genes and disorders have been successfully modeled in the zebrafish, highlighting the striking similarity between zebrafish and human melanocytes. Unlike humans, zebrafish have xanthophore and iridophore pigmentation cells, however in this manuscript we restrict our studies to melanocytes (van Rooijen et al., 2017). Five days after NNT overexpression, a decrease in intramelanocytic pigmentation was observed in NNT-overexpressing zebrafish compared with empty plasmid Zebrafish embryos (FIG. 4B). This observation was confirmed by pixel-based brightness quantification analysis. Deletion of nnt using CRISPR-Cas9 (FIG. 11B) resulted in darkened melanocytes (FIG. 4C). Similar to the genetic deletion of nnt, treatment of zebrafish embryos for 24 hours with the chemical NNT-inhibitors (DCC and 2,3BD), resulted in a significant darkening (FIG. 4D). However, subsequent treatment of NNT overexpressing fish with 2,3 BD prevented the NNT OE-induced decrease in melanocytic pigmentation (FIG. 11C). This finding is in line with previous publications confirming an inhibitory role 2.3BD and DCC on NNT enzyme activity (Phelps and Hatefi, 1981) (Moody and Reid, 1983). Next, we examined the status of NNT in human hyperpigmentation disorders including post inflammatory hyperpigmentation (PIH) and lentigo. Skin biopsies of nine Asian patients were co-stained for NNT and 4′,6-diamidino-2-phenylindole (DAPI) immunofluorescence. NNT intensity was normalized to the sample's DAPI intensity and cell count. Both epidermal and upper dermal skin were investigated. In line with the Human Protein Atlas, NNT is expressed in different epidermal cells including keratinocytes, fibroblasts, and melanocytes (Uhlen et al., 2015), were moderate levels of NNT expression (red) detected throughout the epidermis and upper dermis (FIG. 4E, Left panels). While non-inflammatory skin disorders, such as ABNOM (Acquired, bilateral nevus of Ota-like macules, also known as Hori nevus), displayed NNT expression levels similar to those of healthy skin (data not shown), skin of patients with inflammation-induced disorders displayed decreased NNT expression levels. Disorders where intrinsic inflammation was present, such as post-inflammatory hyperpigmentation, or where extrinsic inflammation was present, such as UV-induced lentigo, NNT expression was significantly lower compared with healthy skin (FIG. 4E, middle and right panels). Interestingly, this trend was further enhanced in areas of hyperpigmentation (FIG. 11D).

Thus, NNT levels appear to be associated with murine and zebrafish pigmentation, as well as human disorders of hyperpigmentation.

### Example 7. Statistical Associations Between Genetic Variants of NNT and Human Skin Pigmentation Variation in Diverse Population Cohorts

### Genetic Associations

To investigate whether NNT plays a role in normal skin pigmentation variation in humans, we examined associations between pigmentation and genetic variants within the ˜1.1 Mb NNT gene region. A meta-analysis was performed to combine P-values from Genome-Wide Association Studies (GWAS) conducted in 4 diverse population cohorts with a total of 462,885 individuals: two Western European cohorts (Rotterdam Study (Jacobs et al., 2015), UK Biobank (Hysi et al., 2018; Loh et al., 2018)), a multi-ethnic Latin American cohort (CANDELA (Adhikari et al., 2019)), and a multi-ethnic cohort from Eastern and Southern Africa (Crawford et al., 2017). In these studies skin pigmentation was measured either quantitatively by reflectometry or by an ordinal system (see Methods). UK Biobank summary statistics were also available for ease of skin tanning (sunburn) and use of sun protection.

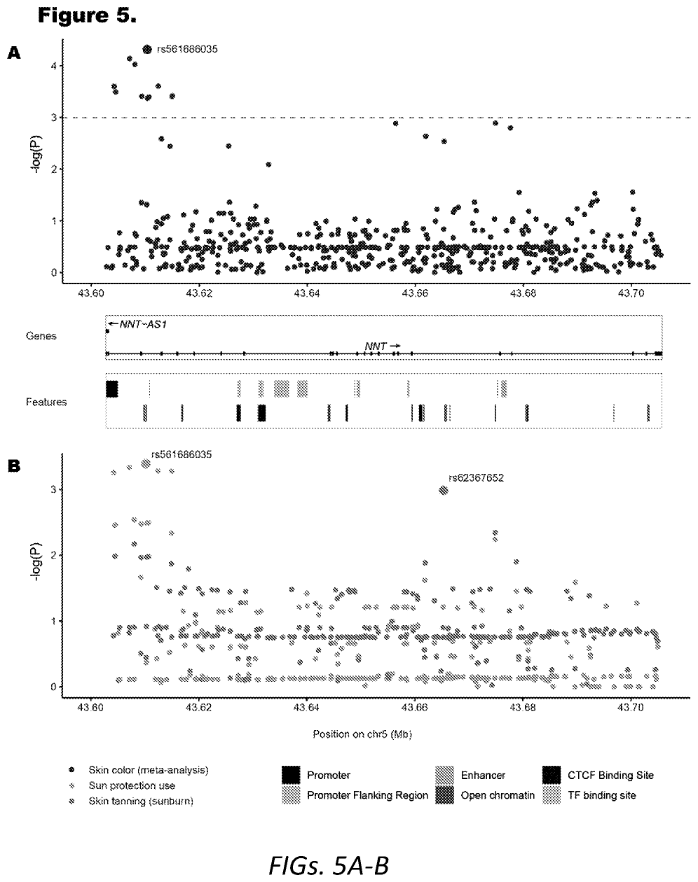

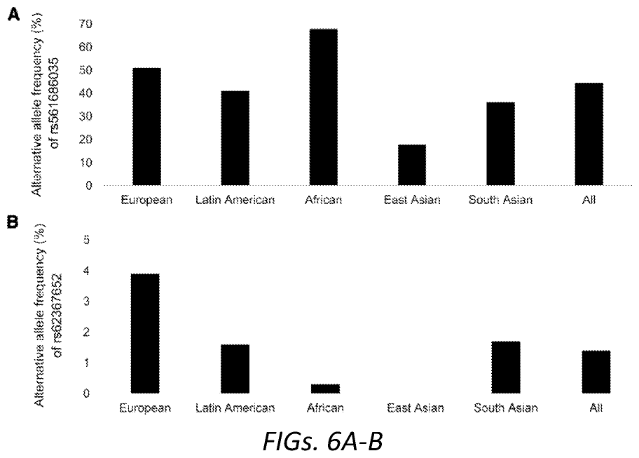

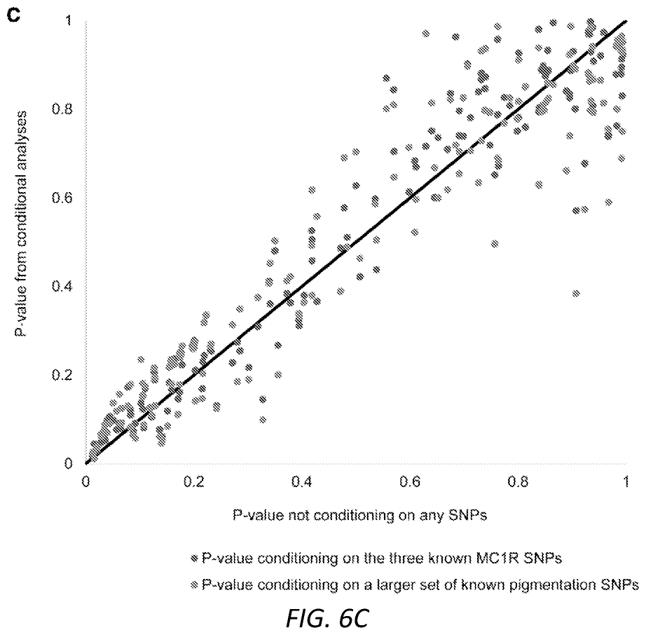

332 variants were available in the combined dataset; using a P-value significance threshold of 1.01E-3 (adjusted for multiple testing, see Methods), 11 variants were significantly associated with skin pigmentation in the meta-analysis (FIG. 5A). The variants were present in all worldwide populations, with the alternative alleles having the highest frequency in Africans (FIG. 6A) and associated with darker skin color. The strongest association (P=4.94E-05) was observed for an intronic variant rs561686035.

It was also the strongest associated variant for sun protection use in the UK Biobank cohort (P=4.15E-04, FIG. 5B), the minor allele being associated with increased use. The UK Biobank cohort also showed a significant association with ease of skin tanning (sunburn), the lowest P-value being 1E-3 for the intronic SNP rs62367652, the minor allele being associated with increased tanning (FIG. 5B, 6B).

### In Silico Expression Analysis of NNT Variants

All the 11 variants that were significant in the meta-analysis of pigmentation are in linkage disequilibrium (LD) (r²>0.7), and they span a 11 KB region at the beginning of the NNT gene overlapping its promoter (ENSR00000180214) (FIG. 5A), which shows regulatory activity in melanocytes and keratinocytes (according to the Ensembl database). Furthermore, several of these variants are highly significant eQTLs for the NNT gene in both sun exposed and unexposed skin tissues (according to the GTEx database). For these variants, the alternative alleles correlated with darker skin color and have negative effect sizes as eQTLs for NNT expression, indicating lower levels of expression of the NNT transcript.

Subsequently, we sought to understand the direction of effect of the NNT genetic variants on these traits and on the expression of NNT. We calculated the correlation between the GWAS effect sizes of the alternative allele of each genetic variant within the NNT region with their effect sizes as eQTLs on the expression of the NNT transcript according to GTEx in the two skin tissues (see Methods). The results are consistent with the direction of association between the NNT transcript expression and skin color as described earlier: expression levels of the NNT transcript in both tissues was negatively correlated with darker skin color (especially in sun unexposed skin tissue, where the effect of external factors such as sunlight is less prominent), and sun protection use (especially in sun exposed skin tissue) as well as sunburn (especially in sun exposed skin tissue).

Therefore, several intronic SNPs within the NNT genomic region were associated with skin pigmentation, tanning, and sun protection use in 4 diverse cohorts including 462,885 individuals. Using eQTL expression data for NNT, we observe that lower expression of the NNT transcript in skin tissues correlates with darker skin color, and consequently less sunburn and less sun protection use.

### Conditioning on Known Pigmentation SNPs

As MC1R is a major determinant of pigmentation, with known genetic variants associated with lighter skin color, red hair, and freckles in European populations (Quillen et al., 2019), we checked whether MC1R can be a confounder in the observed association of NNT with skin pigmentation. In the Western European cohort of the Rotterdam Study, conditioning on the three known MC1R SNPs in the GWAS did not significantly alter the P-values of the NNT variants ((P=0.869, FIG. 6B). Conditioning on a larger set of known pigmentation variants (see Methods) in the GWAS does not significantly alter the P-values of the NNT variants (P=0.191, FIG. 6C) either.

### Example 8. NNT Activators Depigment Melanoma Cells in Viro

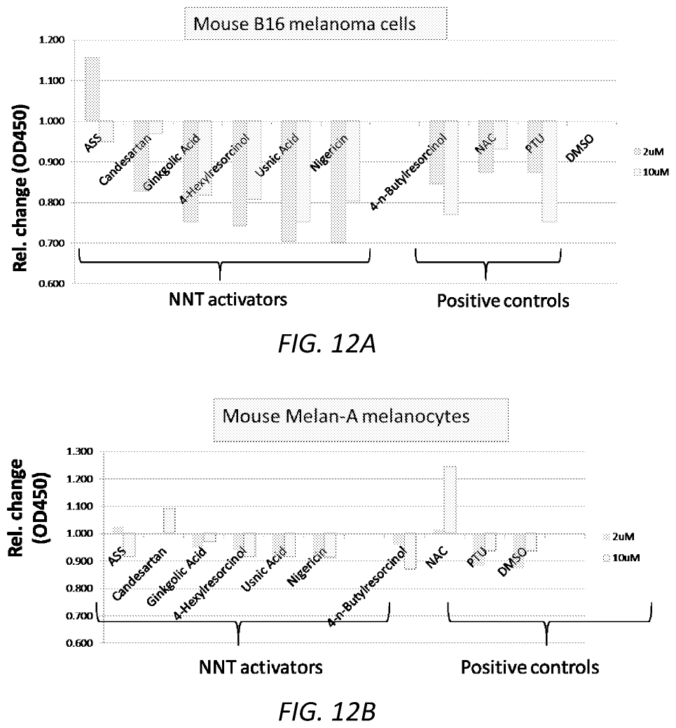

The ability of 2 uM or 10 uM ASS, Usnic acid, 4-hexylresorcinol, candesartan, Nigericin, and Ginkgolic acid to depigment was evaluated in mouse B16 melanoma cells and melan-A melanocytes. A DMSO-based carrier solution was used, and the experiments carried out for 1-5 days, with an average of 1.5 days. 4-n-Butylresorcinol, N-Acetylcystein (NAC), and Phenylthiourea (PTU), were used as a positive control, and DMSO as a negative control. The results, shown in FIGS. 12A-B, demonstrated the depigmenting effects of the tested compounds.

### Example 9. NNT Activators Depigment Skin Explants

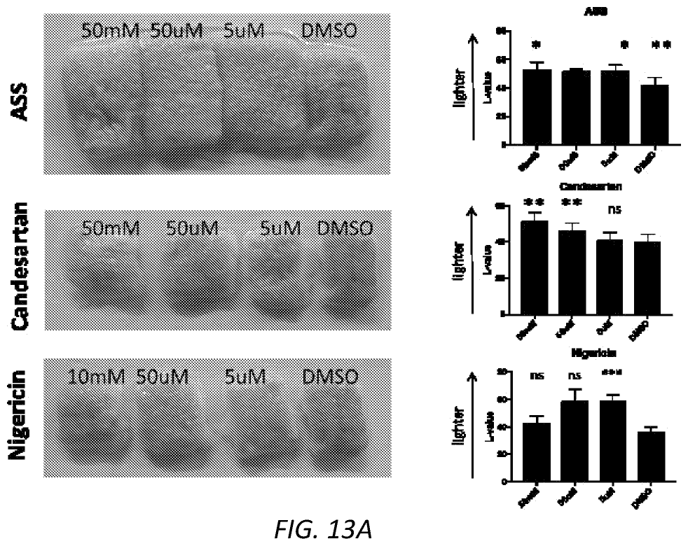

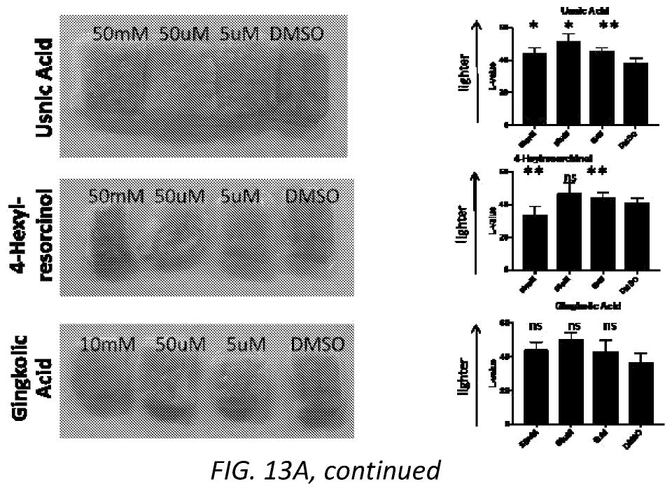

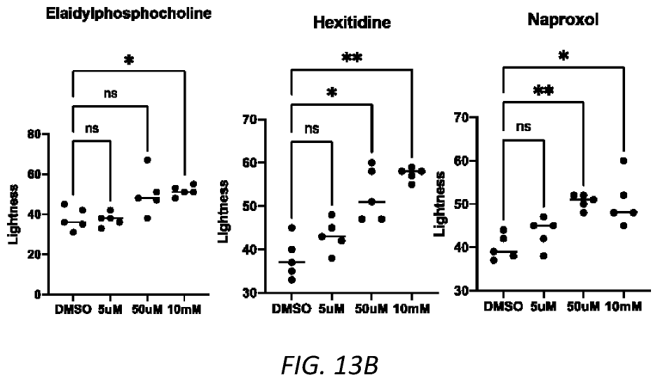

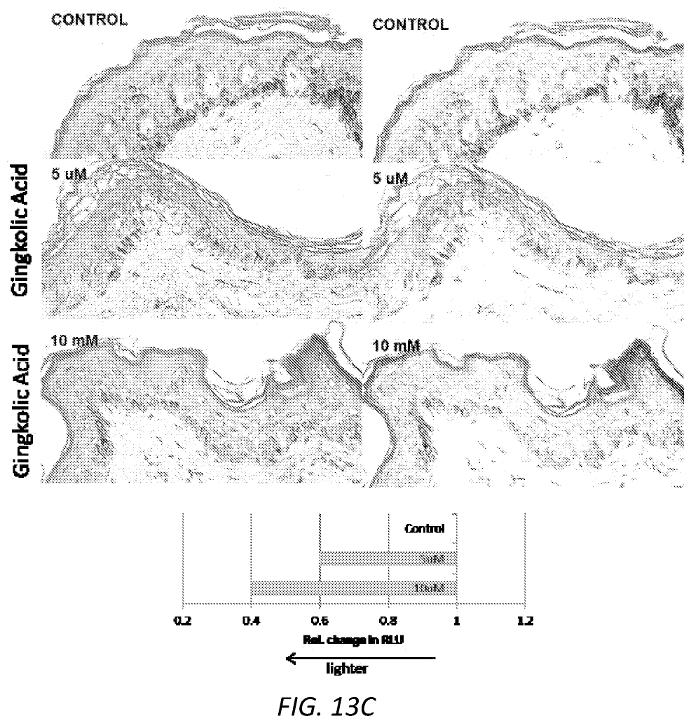

The ability of ASS, Usnic acid, 4-hexylresorcinol, candesartan, Nigericin, elaidylphosphocholine, hexitidine, naproxol, and Ginkgolic acid to depigment skin was tested in human skin explants. A DMSO-based carrier solution was used. DMSO was used as a negative control. The results, shown in FIGS. 13A-B, demonstrated the depigmenting effects of the tested compounds after 36 hours. Table 1 provides exemplary dose ranges.

TABLE 1

| | Dose needed to depigment | |
|---|---|---|
| Treatment | within 36 h (skin type 2) | |
| ASS | 5 | uM-50 mM |
| Usnic Acid | 5 | uM-50 mM |
| Nigericin | 5 | uM |
| Gingkolic Acid | n.s. (est. 50 uM) | |
| Candesartan | 50 | uM-50 mM |
| 4-Heylresorcinol | 5 | uM |

In addition, NNT activator Ginkgolic acid displayed lightening effects in human skin explants, as shown by Fontana Masson and H&E staining (see FIG. 13C). Nuclear capping was present, indicating the presence of proper melanin.

### Example 10. NNT Activators can Prevent UVB-Driven Pigmentation of Skin

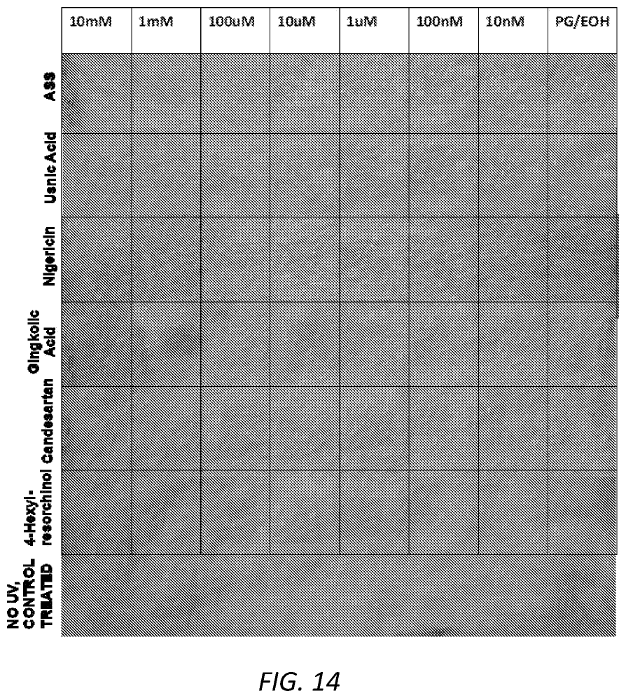

The ability of a number of NNT activators to prevent UVB-driven pigmentation of skin was evaluated in human skin explants (Fitzpatrick skin type 2) with application of UVB 150 mJ/cm². As shown in FIG. 14, the NNT activators tested were able to prevent UVB-driven pigmentation of skin. Table 2 provides exemplary doses useful in inhibiting UVB-induced tanning.

TABLE 2

| | Dose needed to inhibit UVB-induced | |
|---|---|---|
| Treatment | tanning (skin type 2) | |
| ASS | 10 | uM |
| Usnic Acid | 100 | uM |
| Nigericin | 10-100 | uM |
| Gingkolic Acid | 100 | uM |
| Candesartan | 1-10 | mM |
| 4-Heylresorcinol | 1 | mM |

### Example 11. Nnt Activators can Depigment Human Skin

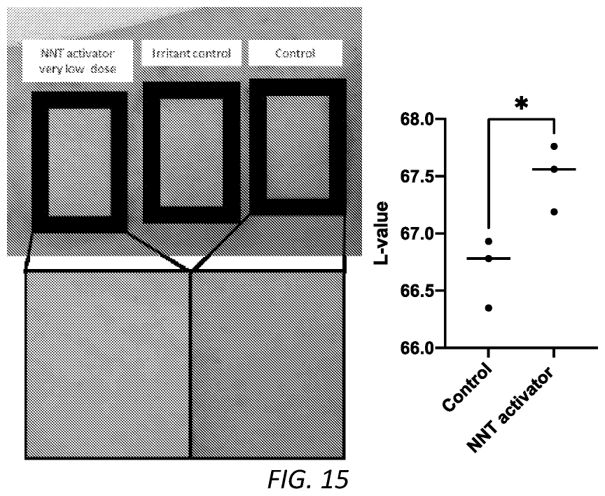

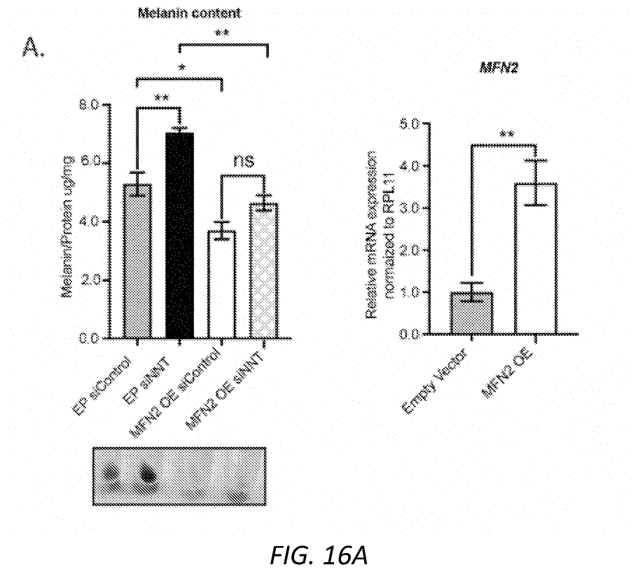

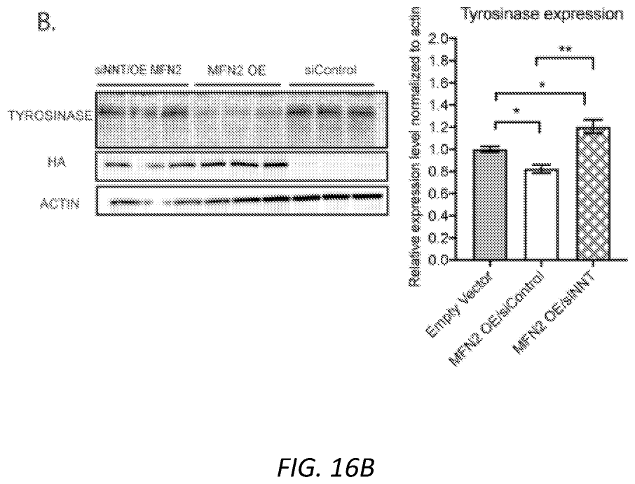

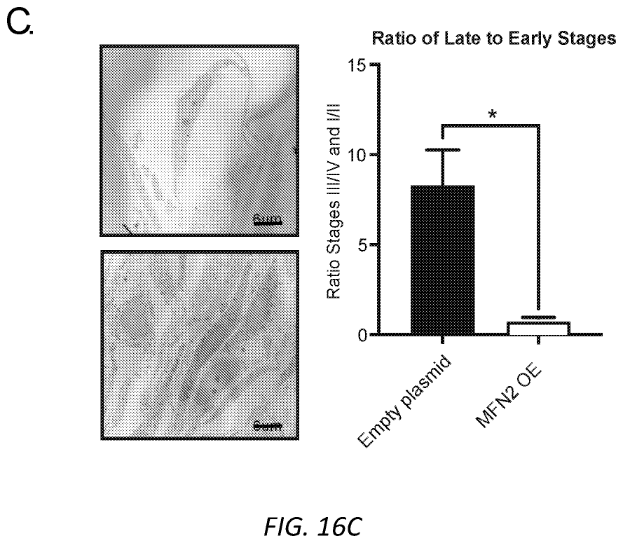

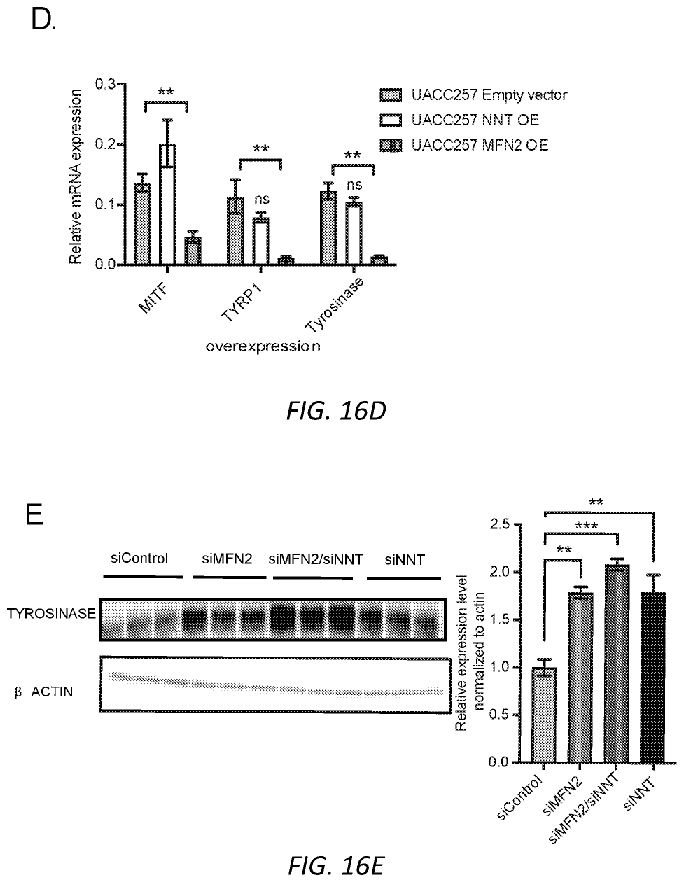

A human subject with skin type 2 was treated with Hexetidine 100 uM in DMSO for 15 days, twice per day, individual. The results, seen in FIG. 15, showed successful depigmentation without significant irritation.

### Example 12. MFNT Agonists Show Depigmenting Effects

As outlined above, we observed that siMFN2 did not change pigmentation in UACC257 melanoma cells. However, siMFN2 suppressed the increase in pigmentation induced by siNNT (FIG. 9F) and MFN2 overexpression induced a significant depigmentation (Figure attached below this reply, Panel A).

Interestingly, similar to overexpression of NNT (FIG. 1F), overexpression of MFN2 led to hypopigmentation (Figure below, Panel A), a significant decrease in tyrosinase protein levels (Figure below, Panel B), and a decrease in melanosome maturation (Figure below, Panel C) in primary human melanocytes. However, in contrast to NNT overexpression (FIG. 1F, 2G and FIG. S2N), MFN2 overexpression was accompanied by a significant decrease in the tyrosinase, TRP1 and MITF mRNA levels (Figure below, Panel D), indicating a possible interference with the MITF pathway. Upon silencing of MFN2, tyrosinase protein increased (Figure below, Panel E). However, siMFN2 alone did not increase pigmentation (FIG. S3F), and electron microscopy revealed that knockdown of MFN2 was accompanied by large autophagosome-like structures containing numerous and partly intact melanosomes (FIG. S31), as well as increased LCB3 Type II (FIG. S3J) [which can be associated with either enhanced autophagosome synthesis or reduced autophagosomes (Barth et al., 2010)], which is consistent with previous reports (Zhao et al., 2012) (Sebastian et al., 2016). This is a striking finding, despite the fact that siMFN2 simultaneously produced diminished pigmentation. We believe these observations are explained (below) by alterations in the fate of the melanosomes in the context of siMFN2.

siNNT significantly increased melanosome maturation (FIG. 2F), while NNT overexpression decreased melanosome maturation (FIG. 2G).

In line with the previously discussed hypothesis of MFN2-driven changes in melanosome-mitochondrial proximity, we also measured melanosome-mitochondrial distances (figure S3C). In siNNT-treated primary melanocytes, a significant increase in the number of close melanosome-mitochondria contacts (<20 nm) was observed and NNT OE decreased the number of close contacts. The combination of siNNT and siMFN2 reversed siNNT-driven increased in dose contacts. As suspected, siMFN2 alone resulted in decreased proximity compared to control.

To summarize, these data suggest that siNNT drives pigmentation via promotion of melanosome maturation, which is associated with stabilization of tyrosinase and tyrosinase-related proteins (TYRP-1 and DCT) (FIG. 2A). The observation that the absolute numbers of melanosomes did not change upon silencing and overexpression of NNT (FIG. S3D) was in line with the observation that Pmel17 (FIG. 2A), a marker for early melanosome development, did not change. These data therefore collectively suggest that NNT regulates melanosome maturation and pigmentation via a redox-dependent process.

### REFERENCES

Human Protein Atlas available from proteinatlas.org.
Ablain, J., Durand, E. M., Yang, S., Zhou, Y., and Zon, L. I. (2015). A CRISPR/Cas9 vector system for tissue-specific gene disruption in zebrafish. Dev Cell 32, 756-764.
Adhikari, K., Mendoza-Revilla, J., Sohail, A., Fuentes-Guajardo, M., Lampert, J., Chacon-Duque, J. C., Hurtado. M., Villegas, V., Granja, V., Acuna-Alonzo, V., et al. (2019). A GWAS in Latin Americans highlights the convergent evolution of lighter skin pigmentation in Eurasia. Nature communications 10, 358.
Allouche, J., Bellon, N., Saidani, M., Stanchina-Chatrousse, L., Masson, Y., Patwardhan, A., Gilles-Marsens, F., Delevoye, C., Domingues, S., Nissan, X., et al. (2015). In vitro modeling of hyperpigmentation associated to neurofibromatosis type 1 using melanocytes derived from human embryonic stem cells. Proc Natl Acad Sci USA 112, 9034-9039.
Arjinpathana, N., and Asawanonda, P. (2012). Glutathione as an oral whitening agent: a randomized, double-blind, placebo-controlled study. J Dermatolog Treat 23, 97-102.
Bae, S., Park, J., and Kim, J. S. (2014). Cas-OFFinder: a fast and versatile algorithm that searches for potential off-target sites of Cas9 RNA-guided endonucleases. Bioinformatics 30, 1473-1475.
Barth, S., Glick, D., and Macleod, K. F. (2010). Autophagy: assays and artifacts. J Pathol 221, 117-124.
Bellei, B., Maresca, V., Flori, E., Pitisci, A., Larue, L., and Picardo, M. (2010). p38 regulates pigmentation via proteasomal degradation of tyrosinase. The Journal of biological chemistry 285, 7288-7299.
Benjamini, Y., and Cohen, R. (2017). Weighted false discovery rate controlling procedures for clinical trials. Biostatistics 18, 91-104.
Bennett, D. C., Cooper, P. J., and Hart, I. R. (1987). A line of non-tumorigenic mouse melanocytes, syngeneic with the B16 melanoma and requiring a tumour promoter for growth. Int J Cancer 39, 414-418.
Berger, M. F., Hodis, E., Heffeman, T. P., Deribe, Y. L., Lawrence, M. S., Protopopov, A., Ivanova, E., Watson, I. R., Nickerson, E., Ghosh, P., et al. (2012). Melanoma genome sequencing reveals frequent PREX2 mutations. Nature 485, 502-506.
Ceol, C. J., Houvras. Y., Jane-Valbuena, J., Bilodeau, S., Orlando, D. A., Battisti, V., Fritsch, L., Lin, W. M., Hollmann, T. J., Ferre, F., et al. (2011). The histone methyltransferase SETDB1 is recurrently amplified in melanoma and accelerates its onset. Nature 471. 513-517.
Cestari, T. F., Dantas, L. P., and Boza, J. C. (2014). Acquired hyperpigmentations. Anais brasileiros de dermatologia 89, 11-25.
Clement, K., Rees, H., Canver, M. C., Gehrke, J. M., Farouni, R., Hsu, J. Y., Cole, M. A., Liu, D. R., Joung, J. K., Bauer, D. E., et al. (2019). CRISPResso2 provides accurate and rapid genome editing sequence analysis. Nat Biotechnol 37, 224-226.
Crawford, N. G., Kelly, D. E., Hansen, M. E. B., Beltrame, M. H., Fan, S., Bowman, S. L., Jewett, E., Ranciaro, A., Thompson, S., Lo, Y., et al. (2017). Loci associated with skin pigmentation identified in African populations. Science 358.
d'Ischia, M., Wakamatsu, K., Napolitano, A., Briganti, S., Garcia-Borron, J. C., Kovacs, D., Meredith, P., Pezzella, A., Picardo, M., Sarna, T., et al. (2013). Melanins and melanogenesis: methods, standards, protocols. Pigment Cell Melanoma Res 26, 616-833.
D′Orazio, J. A., Nobuhisa, T., Cui, R., Arya, M., Spry, M., Wakamatsu, K., Igras, V., Kunisada, T., Granter, S. R., Nishimura, E. K., et al. (2006). Topical drug rescue strategy and skin protection based on the role of Mc1r in UV-induced tanning. Nature 443, 340-344.
Daniele, T., Hurbain, I., Vago, R., Casari, G., Raposo, G., Tacchetti, C., and Schiaffino, M. V. (2014). Mitochondria and melanosomes establish physical contacts modulated by Mfn2 and involved in organelle biogenesis. Curr Biol 24, 393-403.
Del Bino, S., Ito, S., Sok, J., Nakanishi, Y., Bastien, P., Wakamatsu, K., and Bernerd, F. (2015). Chemical analysis of constitutive pigmentation of human epidermis reveals constant eumelanin to pheomelanin ratio. Pigment cell & melanoma research 28, 707-717.
Earle, S. R., and Fisher, R. R. (1980). A direct demonstration of proton translocation coupled to transhydrogenation in reconstituted vesicles. The Journal of biological chemistry 255, 827-830.
Filadi, R., Pendin, D., and Pizzo, P. (2018). Mitofusin 2: from functions to disease. Cell Death Dis 9, 330.
Fuller, B. B., et al., Regulation of the catalytic activity of preexisting tyrosinase in black and Caucasian human melanocyte cell cultures. Exp Cell Res. 2001 Jan 15:262(2):197-208. doi: 10.1006/excr.2000.5092.
Genomes Project, C., Abecasis, G. R., Auton, A., Brooks, L. D., DePristo, M. A., Durbin, R. M., Handsaker, R. E., Kang, H. M., Marth, G. T., and McVean, G. A. (2012). An integrated map of genetic variation from 1,092 human genomes. Nature 491, 56-65.
Ho. H., and Ganesan, A. K. (2011). The pleiotropic roles of autophagy regulators in melanogenesis. Pigment Cell Melanoma Res 24, 595-604.
Hofman, A., Brusselle, G. G., Darwish Murad, S., van Duijn, C. M., Franco, O. H., Goedegebure, A., Ikram, M. A., Klaver, C. C., Nijsten, T. E., Peeters, R. P., et al. (2015). The Rotterdam Study: 2016 objectives and design update. Eur J Epidemiol 30, 661-708.
Horike, N., Kumagai, A., Shimono, Y., Onishi, T., Itoh, Y., Sasaki, T., Kitagawa, K., Hatano, O., Takagi, H., Susumu, T., et al. (2010). Downregulation of SIK2 expression promotes the melanogenic program in mice. Pigment cell & melanoma research 23, 809-819.
Huang, T. T., Naeemuddin, M., Elchuri, S., Yamaguchi, M., Kozy, H. M., Carison, E. J., and Epstein, C. J. (2006). Genetic modifiers of the phenotype of mice deficient in mitochondrial superoxide dismutase. Hum Mol Genet 15, 1187-1194.
Huls, A., Vierkotter, A., Gao, W., Kramer, U., Yang, Y., Ding, A., Stolz, S., Matsui, M., Kan, H., Wang, S., et al. (2016). Traffic-Related Air Pollution Contributes to Development of Facial Lentigines: Further Epidemiological Evidence from Caucasians and Asians. J Invest Dermatol 136, 1053-1056.
Hysi, P. G., Valdes, A. M., Liu, F., Furlotte, N. A., Evans, D. M., Bataille, V., Visconti, A., Hemani, G., McMahon, G., Ring, S. M., et al. (2018). Genome-wide association meta-analysis of individuals of European ancestry identifies new loci explaining a substantial fraction of hair color variation and heritability. Nature genetics 50, 652-656.
Ikram, M. A., Brusselle, G. G. O., Murad, S. D., van Duijn, C. M., Franco, O. H., Goedegebure, A., Klaver, C. C. W., Nijsten, T. E. C., Peeters, R. P., Stricker, B. H., et al. (2017). The Rotterdam Study: 2018 update on objectives, design and main results. Eur J Epidemiol 32, 807-850.
Iozumi, K., Hoganson, G. E., Pennella, R., Everett, M. A., and Fuller, B. B. (1993). Role of tyrosinase as the determinant of pigmentation in cultured human melanocytes. J Invest Dermatol 100, 806-811.
Ito, S., and Ifpcs (2003). The IFPCS presidential lecture: a chemist's view of melanogenesis. Pigment cell research/sponsored by the European Society for Pigment Cell Research and the International Pigment Cell Society 16, 230-236.
Ito, S., Nakanishi, Y., Valenzuela, R. K., Brilliant, M. H., Kolbe, L., and Wakamatsu, K. (2011). Usefulness of alkaline hydrogen peroxide oxidation to analyze eumelanin and pheomelanin in various tissue samples: application to chemical analysis of human hair melanins. Pigment Cell Melanoma Res 24, 605-613.
Jablonski, N. G., and Chaplin, G. (2012). Human skin pigmentation, migration and disease susceptibility. Philos Trans R Soc Lond B Biol Sci 367, 785-792.
Jablonski, N. G., and Chaplin, G. (2017). The colours of humanity: the evolution of pigmentation in the human lineage. Philos Trans R Soc Lond B Biol Sci 372.
Jacobs, L. C., Hamer, M. A., Verkouteren, J. A., Pardo, L. M., Liu, F., and Nijsten, T. (2015). Perceived skin colour seems a swift, valid and reliable measurement. Br J Dermatol 173, 1084-1086.
Jara, J. R., Aroca, P., Solano, F., Martinez, J. H., and Lozano, J. A. (1988). The role of sulfhydryl compounds in mammalian melanogenesis: the effect of cysteine and glutathione upon tyrosinase and the intermediates of the pathway. Biochimica et biophysica acta 967, 296-303.
Jaruga, P., and Dizdaroglu, M. (2008). 8,5′-Cyclopurine-2′-deoxynucleosides in DNA: mechanisms of formation, measurement, repair and biological effects. DNA Repair (Amst) 7, 1413-1425.
Jiang, L., Zheng, Z., Qi, T., Kemper, K. E., Wray, N. R., Visscher, P. M., and Yang, J. (2019). A resource-efficient tool for mixed model association analysis of large-scale data. Nature genetics 51, 1749-1755.
Jones, P., Lucock, M., Veysey, M., and Beckett, E. (2018). The Vitamin D(−) Folate Hypothesis as an Evolutionary Model for Skin Pigmentation: An Update and Integration of Current Ideas. Nutrients 10.
Khaled, M., Levy, C., and Fisher, D. E. (2010). Control of melanocyte differentiation by a MITF-PDE4D3 homeostatic circuit. Genes & development 24, 2276-2281.
King, R., Weilbaecher, K. N., McGill, G., Cooley, E., Mihm, M., and Fisher, D. E. (1999). Microphthalmia transcription factor. A sensitive and specific melanocyte marker for MelanomaDiagnosis. Am J Pathol 155, 731-738.
Kleinstiver, B. P., Sousa, A. A., Walton, R. T., Tak, Y. E., Hsu, J. Y., Clement, K., Welch, M. M., Horng, J. E., Malagon-Lopez, J., Scarfo, I., et al. (2019). Engineered CRISPR-Cas12a variants with increased activities and improved targeting ranges for gene, epigenetic and base editing. Nat Biotechnol 37, 276-282.
Kolb L et al., 4-n-butylresorcinol, a highly effective tyrosinase inhibitor for the topical treatment of hyperpigmentation. J Eur Acad Dermatol Venereol. 2013 January; 27 Suppl 1:19-23.
Labun, K., Montague, T. G., Krause, M., Torres Cleuren, Y. N., Tjeldnes, H., and Valen, E. (2019). CHOPCHOP v3: expanding the CRISPR web toolbox beyond genome editing. Nucleic Acids Res 47, W171-W174.
Lamason, R. L., Mohideen, M. A., Mest, J. R., Wong, A. C., Norton, H. L., Aros, M. C., Jurynec, M. J., Mao, X., Humphreville, V. R., Humbert, J. E., et al. (2005). SLC24A5, a putative cation exchanger, affects pigmentation in zebrafish and humans. Science 310, 1782-1786.
Langendonk, J. G., Balwani, M., Anderson, K. E., Bonkovsky, H. L., Ansley, A. V., Bissell, D. M., Bloomer, J., Edwards, C., Neumann, N.J., Parker, C., et al. (2015). Afamelanotide for Erythropoietic Protoporphyria. The New England journal of medicine 373, 48-59.
Leboucher, G. P., Tsai, Y. C., Yang, M., Shaw, K. C., Zhou, M., Veenstra, T. D., Glickman, M. H., and Weissman, A. M. (2012). Stress-induced phosphorylation and proteasomal degradation of mitofusin 2 facilitates mitochondrial fragmentation and apoptosis. Mol Cell 47, 547-557.
Lin, M., Siford, R. L., Martin, A. R., Nakagome, S., Moller, M., Hoal, E. G., Bustamante, C. D., Gignoux, C. R., and Henn, B. M. (2018). Rapid evolution of a skin-lightening allele in southern African KhoeSan. Proceedings of the National Academy of Sciences of the United States of America 115, 13324-13329.
Lo, J. A., and Fisher, D. E. (2014). The melanoma revolution: from UV carcinogenesis to a new era in therapeutics. Science 346, 945-949.
Loh, P. R., Kichaev, G., Gazal, S., Schoech, A. P., and Price, A. L. (2018). Mixed-model association for biobank-scale datasets. Nature genetics 50, 906-908.
Makino E. T. T, et al., Evaluation of a hydroquinone-free skin brightening product using in vitro inhibition of melanogenesis and clinical reduction of ultraviolet-induced hyperpigmentation. J Drugs Dermatol. 2013 March; 12(3): s16-20.
Martin, A. R., Lin, M., Granka, J. M., Myrick, J. W., Liu, X., Sockell, A., Atkinson, E. G., Werely, C. J., Moller, M., Sandhu, M. S., et al. (2017). An Unexpectedly Complex Architecture for Skin Pigmentation in Africans. Cell 171, 1340-1353 e1314.
Mitra, D., Luo, X., Morgan, A., Wang, J., Hoang, M. P., Lo, J., Guerrero. C. R., Lennerz, J. K.,
Mihm, M. C., Wargo, J. A., et al. (2012). An ultraviolet-radiation-independent pathway to melanoma carcinogenesis in the red hair/fair skin background. Nature 491, 449-453.
Moody, A. J., and Reid, R. A. (1983). Inhibition of nicotinamide nucleotide transhydrogenase in rat liver submitochondrial particles by dicyclohexylcarbodi-imide and butanedione. Biochem J 209, 889-892.
Moreno-Mateos, M. A., Vejnar, C. E., Beaudoin, J. D., Fernandez, J. P., Mis, E. K., Khokha, M. K., and Giraldez, A. J. (2015). CRISPRscan: designing highly efficient sgRNAs for CRISPR-Cas9 targeting in vivo. Nat Methods 12, 982-988.
Mujahid, N., Liang, Y., Murakami, R., Choi. H. G., Dobry, A. S., Wang, J., Suita, Y., Weng, Q. Y., Allouche, J., Kemeny, L. V., et al. (2017). A UV-Independent Topical Small-Molecule Approach for Melanin Production in Human Skin. Cell Rep 19, 2177-2184.
Park, H. Y., Kosmadaki, M., Yaar, M., and Gilchrest, B. A. (2009). Cellular mechanisms regulating human melanogenesis. Cell Mol Life Sci 66, 1493-1506.
Park, S. B., Suh, D. H., and Youn, J. I. (1999). A long-term time course of colorimetric evaluation of ultraviolet light-induced skin reactions. Clin Exp Dermatol 24, 315-320.
Paterson, E. K., Fielder, T. J., MacGregor, G. R., Ito, S., Wakamatsu, K., Gillen, D. L., Eby, V., Boissy, R. E., and Ganesan, A. K. (2015). Tyrosinase Depletion Prevents the Maturation of Melanosomes in the Mouse Hair Follicle. PLoS One 10, e0143702.
Pathak, M. A., Riley, F. J., Fitzpatrick, T. B., and Curwen, W. L. (1962). Melanin formation in human skin induced by long-wave ultra-violet and visible light. Nature 193, 148-150.
Phelps, D. C., and Hatefi, Y. (1981). Inhibition of the mitochondrial nicotinamide nucleotide transhydrogenase by dicyclohexylcarbodiimide and diethylpyrocarbonate. J Biol Chem 256, 8217-8221.
Premi, S., Wallisch, S., Mano, C. M., Weiner. A. B., Bacchiocchi, A., Wakamatsu. K., Bechara, E. J., Halaban, R., Douki, T., and Brash, D. E. (2015). Photochemistry. Chemiexcitation of melanin derivatives induces DNA photoproducts long after UV exposure. Science 347, 842-847.
Purcell, S., Neale, B., Todd-Brown, K., Thomas, L., Ferreira, M. A., Bender, D., Mailer, J., Sklar, P., de Bakker, P. I., Daly, M. J., et al. (2007). PLINK: a tool set for whole-genome association and population-based linkage analyses. Am J Hum Genet 81, 559-575.
Quillen, E. E., Norton, H. L., Parra, E. J., Lona-Durazo, F., Ang, K. C., Illiescu, F. M., Pearson, L. N., Shriver, M. D., Lasisi, T., Gokcumen, O., et al. (2019). Shades of complexity: New perspectives on the evolution and genetic architecture of human skin. Am J Phys Anthropol 168 Suppl 67, 4-26.
Rachmin, I., Ostrowski, S. M., Weng, Q. Y., and Fisher, D. E. (2020). Topical treatment strategies to manipulate human skin pigmentation. Adv Drug Deliv Rev 153, 65-71.
Raposo, G., and Marks, M. S. (2007). Melanosomes--dark organelles enlighten endosomal membrane transport. Nat Rev Mol Cell Biol 8, 786-797.
Rendon, M. I., and Gaviria, J. I. (2005). Review of skin-lightening agents. Dermatologic surgery: official publication for American Society for Dermatologic Surgery [et al] 31, 886-889; discussion 889.
Rigler, M. W., and Longo. W. E. (2010). Emission of diacetyl (2,3 butanedione) from natural butter, microwave popcorn butter flavor powder, paste, and liquid products. Int J Occup Environ Health 16, 291-302.
Rohland, N., and Reich, D. (2012). Cost-effective, high-throughput DNA sequencing libraries for multiplexed target capture. Genome Res 22, 939-946.
Roider, E. M. and Fisher, D. E. METHODS AND COMPOSITIONS FOR ENHANCING SKIN PIGMENTATION. WO/2016/077817 (2016).
Ronchi, J. A., Figueira, T. R., Ravagnani, F. G., Oliveira, H. C., Vercesi, A. E., and Castilho, R. F. (2013). A spontaneous mutation in the nicotinamide nucleotide transhydrogenase gene of C57BL/6J mice results in mitochondrial redox abnormalities. Free Radic Biol Med 63, 446-456.
Ronchi, J. A., Francisco, A., Passos, L. A., Figueira, T. R., and Castilho, R. F. (2016). The Contribution of Nicotinamide Nucleotide Transhydrogenase to Peroxide Detoxification Is Dependent on the Respiratory State and Counterbalanced by Other Sources of NADPH in Liver Mitochondria. The Journal of biological chemistry 291, 20173-20187.
Rydstrom, J. (1972). Site-specific inhibitors of mitochondrial nicotinamide-nucleotide transhydrogenase. Eur J Biochem 31, 496-504.
Rydstrom, J., Teixeira da Cruz, A., and Ernster, L. (1970). Factors governing the steady state of the mitochondrial nicotinamide nucleotide transhydrogenase system. The Biochemical journal 116, 12P-13P.
Sancak, Y., Peterson, T. R., Shaul, Y. D., Lindquist, R. A., Thoreen, C. C., Bar-Peled, L., and Sabatini, D. M. (2008). The Rag GTPases bind raptor and mediate amino acid signaling to mTORC1. Science 320, 1496-1501.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Sebastian, D., Sorianello, E., Segales, J., Irazoki, A., Ruiz-Bonilla, V., Sala, D., Planet, E., Berenguer-Llergo, A., Munoz, J. P., Sanchez-Feutrie, M., et al. (2016). Mfn2 deficiency links age-related sarcopenia and impaired autophagy to activation of an adaptive mitophagy pathway. EMBO J 35, 1677-1693.
Sheeran, F. L., Rydstrom, J., Shakhparonov, M. L., Pestov, N. B., and Pepe, S. (2010). Diminished NADPH transhydrogenase activity and mitochondrial redox regulation in human failing myocardium. Biochimica et biophysica acta 1797, 1138-1148.
Siegel, R. L., Miller, K. D., and Jemal, A. (2019). Cancer statistics, 2019. CA Cancer J Clin 69, 7-34.
Sonthalia, S., Daulatabad, D., and Sarkar, R. (2016). Glutathione as a skin whitening agent: Facts, myths, evidence and controversies. Indian journal of dermatology, venereology and leprology 82, 262-272.
Steingrimsson, E., Copeland, N. G., and Jenkins, N. A. (2004). Melanocytes and the microphthalmia transcription factor network. Annu Rev Genet 38, 365-411.
Taylor, A., Pawaskar, M., Taylor, S. L., Balkrishnan, R., and Feldman, S. R. (2008). Prevalence of pigmentary disorders and their impact on quality of life: a prospective cohort study. J Cosmet Dermatol 7. 164-168.
Toye, A. A., Lippiat, J. D., Proks, P., Shimomura, K., Bentley, L., Hugill, A., Mijat, V., Goldsworthy, M., Moir, L., Haynes, A., et al. (2005). A genetic and physiological study of impaired glucose homeostasis control in C57BL/6J mice. Diabetologia 48, 675-686.
Ugwu, S. O., Blanchard, J., Dorr, R. T., Levine, N., Brooks, C., Hadley, M. E., Aickin, M., and Hruby, V. J. (1997). Skin pigmentation and pharmacokinetics of melanotan-I in humans. Biopharm Drug Dispos 18, 259-269.
Uhlen, M., Fagerberg, L., Hallstrom, B. M., Lindskog, C., Oksvold, P., Mardinoglu, A., Sivertsson, A., Kampf, C., Sjostedt, E., Asplund, A., et al. (2015). Proteomics. Tissue-based map of the human proteome. Science 347, 1260419.
van Rooijen, E., Fazio, M., and Zon, L. I. (2017). From fish bowl to bedside: The power of zebrafish to unravel melanoma pathogenesis and discover new therapeutics. Pigment Cell Melanoma Res 30, 402-412.
Vazquez, F., Lim, J. H., Chim, H., Bhalla, K., Gimun, G., Pierce, K., Clish, C. B., Granter, S. R., Widlund, H. R., Spiegelman, B. M., et al. (2013). PGC1alpha expression defines a subset of human melanoma tumors with increased mitochondrial capacity and resistance to oxidative stress. Cancer Cell 23, 287-301.
Wakamatsu, K., Ito, S., and Rees, J. L. (2002). The usefulness of 4-amino-3-hydroxyphenylalanine as a specific marker of pheomelanin. Pigment cell research/sponsored by the European Society for Pigment Cell Research and the International Pigment Cell Society 15, 225-232.
Walton, R. T., Christie, K. A., Whittaker, M. N., and Kleinstiver, B. P. (2020). Unconstrained genome targeting with near-PAMless engineered CRISPR-Cas9 variants. Science 368, 290-296.
Wang, Y. (2008). Bulky DNA lesions induced by reactive oxygen species. Chem Res Toxicol 21, 276-281.
Won, S., Morris, N., Lu, Q., and Elston, R. C. (2009). Choosing an optimal method to combine P-values. Stat Med 28, 1537-1553.
Wu, X., and Hammer, J. A. (2014). Melanosome transfer: it is best to give and receive. Curr Opin Cell Biol 29, 1-7.
Yang, J., Lee, S. H., Goddard, M. E., and Visscher, P. M. (2011). GCTA: a tool for genome-wide complex trait analysis. Am J Hum Genet 88, 76-82.
Yin, F., Sancheti. H., and Cadenas, E. (2012). Silencing of nicotinamide nucleotide transhydrogenase impairs cellular redox homeostasis and energy metabolism in PC12 cells.
Biochimica et biophysica acta 1817, 401-409.
Zhang, Q., Padayatti, P. S., and Leung, J. H. (2017). Proton-Translocating Nicotinamide Nucleotide Transhydrogenase: A Structural Perspective. Front Physiol 8, 1089.
Zhao, T., Huang, X., Han, L., Wang, X., Cheng, H., Zhao, Y., Chen, Q., Chen, J., Cheng, H., Xiao, R., et al. (2012). Central role of mitofusin 2 in autophagosome-lysosome fusion in cardiomyocytes. J Biol Chem 287, 23615-23625.
Zhao, W. N., and McAlister-Henn, L. (1996). Assembly and function of a cytosolic form of NADH-specific isocitrate dehydrogenase in yeast. The Journal of biological chemistry 271, 10347-10352.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of decreasing pigmentation in the skin, hair, and/or eye of a subject, said method comprising administering to the skin, hair, and/or eye of a subject an effective amount of a composition comprising an effective amount of an nicotinamide nucleotide transhydrogenase (NNT) activator and/or a Mitofusin 2 (MFN2) activator.

2. The method of claim 1, wherein the subject has a pigmentation disorder, or wishes to decrease the pigmentation in their skin, hair, and/or eye for cosmetic reasons.

3. The method of claim 2, wherein the pigmentation disorder is a localized skin disorder, optionally benign pigmented skin lesions, such as melanocytic nevi, seborrheic keratosis, lentigines, cafe au lait macules, ephelides, congenital dermal melanocytosis; skin cancers, such as melanoma and pigmented basal cell carcinoma; post-inflammatory pigmentation due to prior injury, current or prior inflammatory skin disease such as eczema, especially in dark-skinned individuals, or fixed drug eruption; current or previous superficial skin infection, particularly pityriasis versicolor and erythrasma; chronic pigmentary disorders, particularly melasma and acquired dermal macular hyperpigmentation; phytophotodermatitis or photocontact dermatitis; thickened skin; or a generalized skin disorder, optionally incontinetia pigmenti, Dowling-Degos-syndrome, metabolic and secondary hyperpigmentation; hyperpigmentation in subjects with Addison's disease, haemochromatosis; metastatic melanoma: diffuse melanosis cutis; and in subjects treated with afamelanotide.

4. A method of decreasing or reducing risk of UVB and/or UVA-induced pigmentation in the skin of a subject in need thereof, said method comprising administering to the skin of a subject in need thereof an effective amount of a composition comprising an effective amount of an NNT activator and/or MFN2 activator, prior to, during, and/or after UVB and/or UVA exposure.

5. The method of claim 1, wherein the composition comprises an NNT activator, preferably usnic acid, elaidylphosphocholine, diplosalsalate, hexylresorcinol, hexetidine, candesartan, Nigericin, Naproxol, or Ginkgolic acid.

6. The method of claim 1, wherein the composition comprises a MFN2 activator, preferably CpdA and CpdB and derivatives thereof, including Chimera B-A/long (B-A/1); 6-Phenylhexanamide derivatives including derivatives of trans-4-hydroxycyclohexyl)-6-phenylhexanamide such as N-(4-hydroxycyclohexyl)-6-phenylhexanamide (MiM111); Leflunomide; echinacoside (ECH); or minipeptide 1 (MP1).

7. The method of claim 1, wherein the composition is a sunscreen, milk, mask, serum, ointment, paste, cream, lotion, gel, powder, solution, spray, or patch.

8. The method of claim 7, wherein the composition comprises dimethyl sulfoxide (DMSO).

9.-16. (canceled)

17. A composition for topical application comprising a nicotinamide nucleotide transhydrogenase (NNT) activator and/or a Mitofusin 2 (MFN2) activator wherein the composition is a sunscreen, milk, mask, serum, ointment, paste, cream, lotion, gel, powder, solution, spray, or patch.

18. The composition of claim 17, wherein the composition comprises an NNT activator, preferably usnic acid, elaidylphosphocholine, diplosalsalate, hexylresorcinol, hexetidine, candesartan, Nigericin, Naproxol, or Ginkgolic acid.

19. The composition of claim 17, wherein the composition comprises a MFN2 activator, preferably CpdA and CpdB and derivatives thereof, including Chimera B-A/long (B-A/l); 6-Phenylhexanamide derivatives including derivatives of trans-4-hydroxycyclohexyl)-6-phenylhexanamide such as N-(4-hydroxycyclohexyl)-6-phenylhexanamide (MiM111); Leflunomide; echinacoside (ECH); or minipeptide 1 (MP1).

20. The composition of claim 17, wherein the composition comprises dimethyl sulfoxide (DMSO).


SEQUENCE LISTING (24 sequences)

SEQ ID NO: 1 - DNA, 74 bp, synthetic construct
  source 1..74: mol_type = other DNA
ctagctagcccgccaccatgtacccatacgatgttccagattacgctgcaaacctattgaaaacagtggtgact

SEQ ID NO: 2 - DNA, 39 bp, synthetic construct
  source 1..39: mol_type = other DNA
ctagctagcttacttctgataggattctctaactttcgc

SEQ ID NO: 3 - DNA, 39 bp, synthetic construct
  source 1..39: mol_type = other DNA
ctagctagcgccaccatgtccctgctcttctctcgatgc

SEQ ID NO: 4 - DNA, 36 bp, synthetic construct
  source 1..36: mol_type = other DNA
ctagctagcttaggatccagcagcgtaatctggaac

SEQ ID NO: 5 - DNA, 20 bp, synthetic construct
  source 1..20: mol_type = other DNA
gttggggagagtggagacag

SEQ ID NO: 6 - DNA, 20 bp, synthetic construct
  source 1..20: mol_type = other DNA
tgccaaaggatctgacagtg

SEQ ID NO: 7 - DNA, 25 bp, synthetic construct
  source 1..25: mol_type = other DNA
cattgttatgctggaaatgctagaa

SEQ ID NO: 8 - DNA, 23 bp, synthetic construct
  source 1..23: mol_type = other DNA
ggcttgctgtatgtggtacttgg

SEQ ID NO: 9 - DNA, 24 bp, synthetic construct
  source 1..24: mol_type = other DNA
accgggaatcctacatggttcctt

SEQ ID NO: 10 - DNA, 24 bp, synthetic construct
  source 1..24: mol_type = other DNA
atgaccagatccgactcgcttgtt

SEQ ID NO: 11 - DNA, 20 bp, synthetic construct
  source 1..20: mol_type = other DNA
agctcaataccccattgctg

SEQ ID NO: 12 - DNA, 20 bp, synthetic construct
  source 1..20: mol_type = other DNA
cacattaagctgaccaggca

SEQ ID NO: 13 - DNA, 20 bp, synthetic construct
  source 1..20: mol_type = other DNA
gtcgtcatgcttatggggat

SEQ ID NO: 14 - DNA, 20 bp, synthetic construct
  source 1..20: mol_type = other DNA
cttttgggttccgtcacttg

SEQ ID NO: 15 - DNA, 19 bp, synthetic construct
  source 1..19: mol_type = other DNA
ctgctaaggaggtgctcaa

SEQ ID NO: 16 - DNA, 22 bp, synthetic construct
  source 1..22: mol_type = other DNA
tcctcacttgaaagccttctgc

SEQ ID NO: 17 - DNA, 20 bp, synthetic construct
  source 1..20: mol_type = other DNA
ctgctagcaagtttgcctca

SEQ ID NO: 18 - DNA, 20 bp, synthetic construct
  source 1..20: mol_type = other DNA
agtggtgcagtgaccaatca

SEQ ID NO: 19 - DNA, 20 bp, synthetic construct
  source 1..20: mol_type = other DNA
aagaggctagaggtcatcag

SEQ ID NO: 20 - DNA, 20 bp, synthetic construct
  source 1..20: mol_type = other DNA
agaacgccatcatcaagaac

SEQ ID NO: 21 - DNA, 21 bp, synthetic construct
  source 1..21: mol_type = other DNA
ccagtcaccaacacagaaatg

SEQ ID NO: 22 - DNA, 20 bp, synthetic construct
  source 1..20: mol_type = other DNA
gtgcaaccagtaacaaagcg

SEQ ID NO: 23 - DNA, 20 bp, synthetic construct
  source 1..20: mol_type = other DNA
ttctcacatcaaggacctgc

SEQ ID NO: 24 - DNA, 20 bp, synthetic construct
  source 1..20: mol_type = other DNA
acacatcacactcgttcctc